(19) United States
(12) Patent Application Publication
(10) Pub. No.: US 2025/0022543 A1
(43) Pub. Date: Jan. 16, 2025

(54) COMPOSITIONS, SYSTEMS, AND METHODS FOR DATA STORAGE USING NUCLEIC ACIDS AND POLYMERASES

(71) Applicant: NAIO, Inc., San Carlos, CA (US)

(72) Inventor: Eric Kool, Stanford, CA (US)

(21) Appl. No.: 18/612,234

(22) Filed: Mar. 21, 2024

Related U.S. Application Data
(63) Continuation of application No. PCT/US2022/076976, filed on Sep. 23, 2022.
(60) Provisional application No. 63/248,407, filed on Sep. 24, 2021.

(51) Int. Cl. G16B 50/30

(52) U.S. Cl. CPC G16B 50/30

(57) ABSTRACT

Nucleic acid polymers for data storage and related methods are provided. In some embodiments, the nucleic acid polymers are writable for data, and in other embodiments, the nucleic acid polymers are encoded with data when synthesized. Generally, a writable polymer may contain one or more convertible residues (e.g., chemically alterable group) bits that are enabled to provide a data code. Various methods can be utilized to generate a writable nucleic acid polymer, or a data encoded nucleic acid polymer. Various methods can be utilized to encode a nucleic acid polymer by selectively modifying convertible residues via a light or redox source with an enzyme (e.g., polymerase) conjugated to a sensitizer. Various methods of reading an encoded nucleic acid polymer are also described herein.

### CROSS REFERENCE TO RELATED APPLICATION

This application is a continuation application of International Patent Application No. PCT/US2022/076976, filed on Sep. 23, 2022, which claims the benefit of U.S. Provisional Patent Application No. 63/248,407, filed on Sep. 24, 2021, each of which is hereby incorporated by reference in its entirety.

### BACKGROUND

In addition to its well-known biological activities, DNA is also under investigation for its ability to store digital information. DNA is inherently digital by nature, with the varying sequence of bases encoding biological data. Scientists and engineers, inspired by this, are working on strategies for encoding digital data in DNA (see, e.g., L. Ceze, J. Nivala, and K. Strauss, Nat Rev Genet. 2019:20:456-466). A common approach to achieve this is to use chemical or biochemical methods to synthesize or assemble strands of DNA of arbitrary sequence that encodes data. After the data is encoded, one can sequence the DNA to obtain or recover the data. Appealing aspects of DNA-based data storage include the possibility of achieving very high density of data storage, since multiple bits can be included in one molecule (strand). A second advantage of the technology is stability, since DNA can maintain sequence information for decades, or centuries, or longer, while current electronic and magnetic storage is not indefinitely stable and requires re-writing.

Although nucleic acids are a great potential source of data storage, the process of synthesizing of nucleic acids in particular data-defining sequences is inefficient and thus the process of encoding the nucleic acids is a substantial barrier to utilizing nucleic acids as data storage. Current approaches for storing data in DNA involve chemical or enzymatic synthesis of strands of arbitrary sequences that encode digital information (see G. M. Church, Y. Gao, and S. Kosuri Science. 2012; 337; 1628; X. Chengtao, et al., Nucleic Acids Res. 2021; 49; 5451-5469; and E. Yoo, et al., Comput Struct Biotechnol J. 2021:19:2468-2476). Oligonucleotide synthesizers can produce DNAs of length up to roughly 100-200 nucleotides. Specialized synthesizers can produce hundreds or thousands of oligonucleotides at one time, which promises higher throughput of data writing. In addition to chemical DNA synthesis, enzymatic approaches involving polymerases or other enzymes are also under investigation for creating DNAs of arbitrary data-encoding sequence. These involve adding specialized nucleotides one at a time, or short segments of DNA step by step.

The approach of encoding data in DNA during synthesis is limited by yield, strand length, time, and cost. Current efficient DNA synthesizers produce strands up to roughly 200 nucleotides, and thus encode relatively small amounts of information per molecule, limiting density. Large numbers of different oligonucleotides must be synthesized to compensate for the short sequences. Oligonucleotide synthesis requires excess reagents to achieve high stepwise yields and requires expensive consumption of reagents and solvents. It also requires time to achieve these high yields for each nucleotide addition (commonly 1-5 min for each step), which implies the need for extended time for encoding larger amounts of data. Common enzymatic approaches under development similarly add nucleotides or groups of nucleotides in stepwise fashion and have not yet greatly improved on the ability to produce very long strands and encode large amounts of data. Because the enzymatic approaches also occur stepwise, they also have limits in the speed of data encoding. Further, since both the above chemical and enzymatic nucleotide addition strategies typically produce relatively short strands, they may not be ideal for single molecule sequencing, and instead may rely on sequencing methods that require larger amounts of each written DNA.

### SUMMARY

Various embodiments are directed to compositions and systems of nucleic acid data storage, modified polymerases for data writing, methods of use thereof, and methods of synthesis thereof. In several embodiments, writable nucleic acid polymers are generated, which can be several thousands of bases long, which can contain repeating convertible residues attached to residues of the nucleic acid polymer. In many embodiments, data is written into a nucleic acid via chemical alteration of the linked convertible residues using a conjugated polymerase together with light or redox signals. In some embodiments, a sensitizer group is conjugated to the polymerase to promote local chemical alteration of the linked convertible residues. In some embodiments, nucleic acids having a written code via nucleobase alteration are stored and/or archived. In some embodiments, nucleic acids having a written code via nucleobase alteration are read via a sequencing apparatus capable of detecting the chemical alterations.

In one aspect, provided herein are nucleic acid polymers for encoding data, comprising: a plurality of convertible residues iteratively spaced along and covalently linked to the backbone of the nucleic acid polymer, wherein each of the plurality of convertible residues has a first state and is capable of being converted from the first state into a second state, the first state and the second state being different, wherein the plurality of convertible residues are covalently linked to the nucleic acid polymer in the first and in the second state, and wherein the nucleic acid polymer comprises a sequence at the 3′ end of the nucleic acid polymer for priming a polymerase.

In some embodiments, the nucleic acid polymer is a single-stranded nucleic acid polymer.

In some embodiments, the sequence is a unique sequence only present at the 3′ end of the nucleic acid polymer.

In some embodiments, the nucleic acid polymer comprises Deoxyribonucleic acid (DNA), Ribonucleic acid (RNA), phosphorothioate DNA, glycerol nucleic acids (GNA), threose nucleic acids (TNA), locked nucleic acids (LNA), or a combination thereof.

In some embodiments, the nucleic acid polymer comprises greater than 10 convertible residues.

In some embodiments, the ratio of the total number of nucleotides of the convertible residues in the nucleic acid polymer is between 2 to 100.

In some embodiments, the plurality of convertible residues are non-naturally occurring nucleobases.

In some embodiments, the plurality of convertible residues are modified naturally occurring nucleobases or derivatives of naturally occurring nucleobases.

In some embodiments, each of the plurality of convertible residues comprises a chemically modifiable moiety.

In some embodiments, each of the plurality of convertible residues, the chemically modifiable moiety is directly attached to the base of the convertible residue.

In some embodiments, each of the plurality of convertible residues the chemically modifiable moiety is attached to the base without a linker or a sidechain.

In some embodiments, the plurality of convertible residues are covalently linked to the backbone of the nucleic acid polymer via the sugar.

In some embodiments, the chemically modifiable moiety is activatable by light, voltage, enzymatic agent, chemical reagent, or a redox agent, thereby converting from the first state into the second state.

In some embodiments, the chemically modifiable moiety is activatable by light, thereby converting from the first state into the second state.

In some embodiments, the conversion from the first state into the second state occurs via an irreversible reaction.

In some embodiments, the convertible residues becomes a naturally occurring nucleobase after conversion into the second state.

In some embodiments, the chemically modifiable moiety is a modifiable fluorophore and the nucleic acid polymer comprises a plurality of modifiable fluorophores.

In some embodiments, the modifiable fluorophores comprises caged fluorophores capable of being converted to uncaged fluorophores by light.

In some embodiments, the modifiable fluorophores comprise photoconvertible fluorophores, wherein the photoconvertible fluorophores exist in a first structural state having a first emission wavelength and are capable of being converted into a second structural state having a second emission wavelength via the light pulses.

In some embodiments, the conversion of the photoconvertible fluorophores from the first structural state into a second structural state is via light pulses at a first wavelength; and wherein the photoconvertible fluorophores are capable of being converted into a third structural state having a third emission wavelength via light pulses at a second wavelength.

In some embodiments, the photoconvertible fluorophores are activated by light in the presence of an additive.

In some embodiments, the photoconvertible fluorophores are inactivated by light.

In some embodiments, the photoconvertible fluorophores are inactivated by light in the presence of an additive.

In some embodiments, the photoconvertible fluorophore comprises a polymethine cyanine dye.

In some embodiments, the plurality of modifiable fluorophores comprise releasable fluorophores that are capable of being released from the polymer by light.

In some embodiments, the plurality of modifiable fluorophores comprise photobleachable fluorophores that are capable of being bleached by light.

In some embodiments, the nucleic acid polymer comprises two or more different sets of convertible residues, each set of convertible residues has a first state and is capable of being converted from the first state into a second state, the first state and the second state being different.

In some embodiments, each of the plurality of convertible residues comprises a chemically modifiable moiety that can be activated by light.

In some embodiments, the two or more different sets of convertible residues are activatable by light of different wavelengths.

In some embodiments, a first set of convertible residues is activatable by light of a first wavelength, and a second set of convertible residues is activatable by light of a second wavelength, the first wavelength and the second wavelength being different.

In some embodiments, the chemically modifiable moiety comprises one or more photo-removable groups.

In some embodiments, the chemically modifiable moiety is a leaving group.

In some embodiments, the convertible residues are selected from

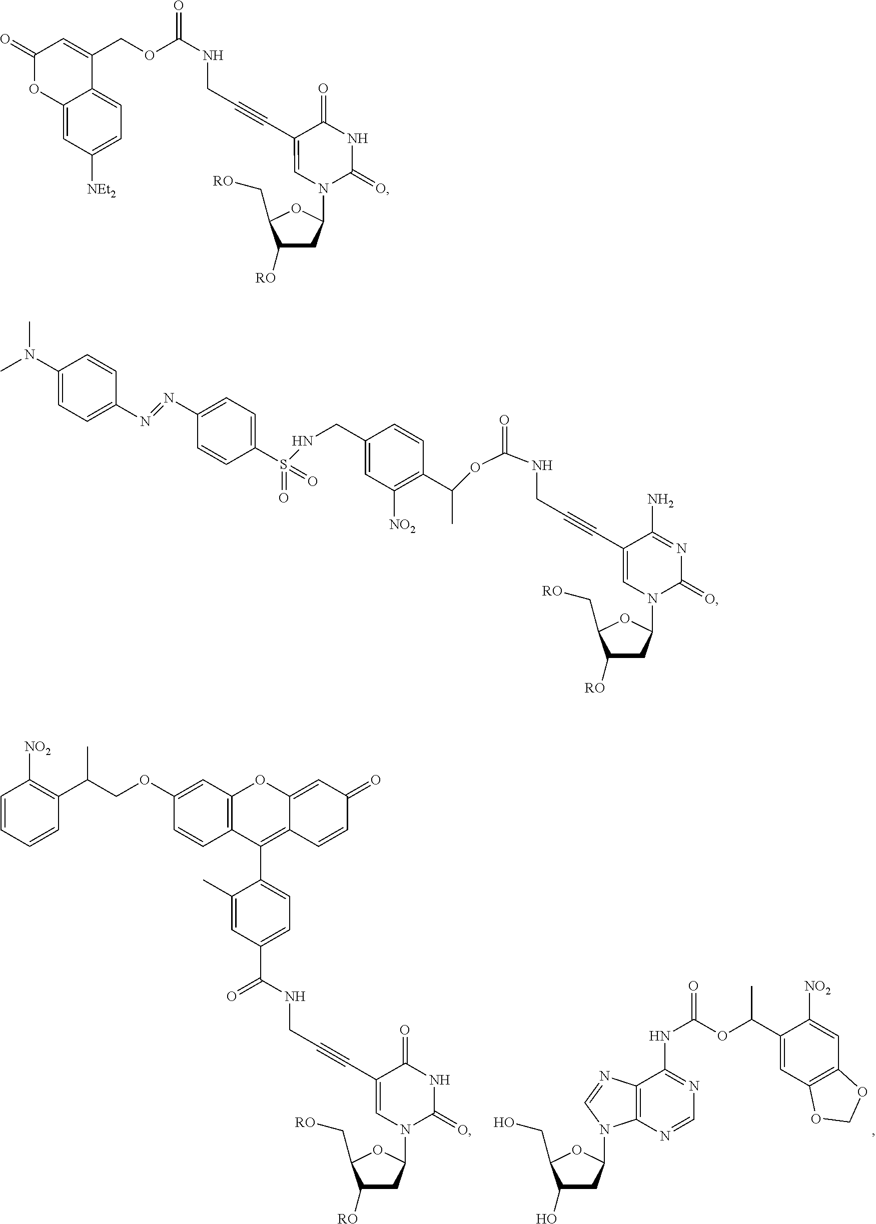

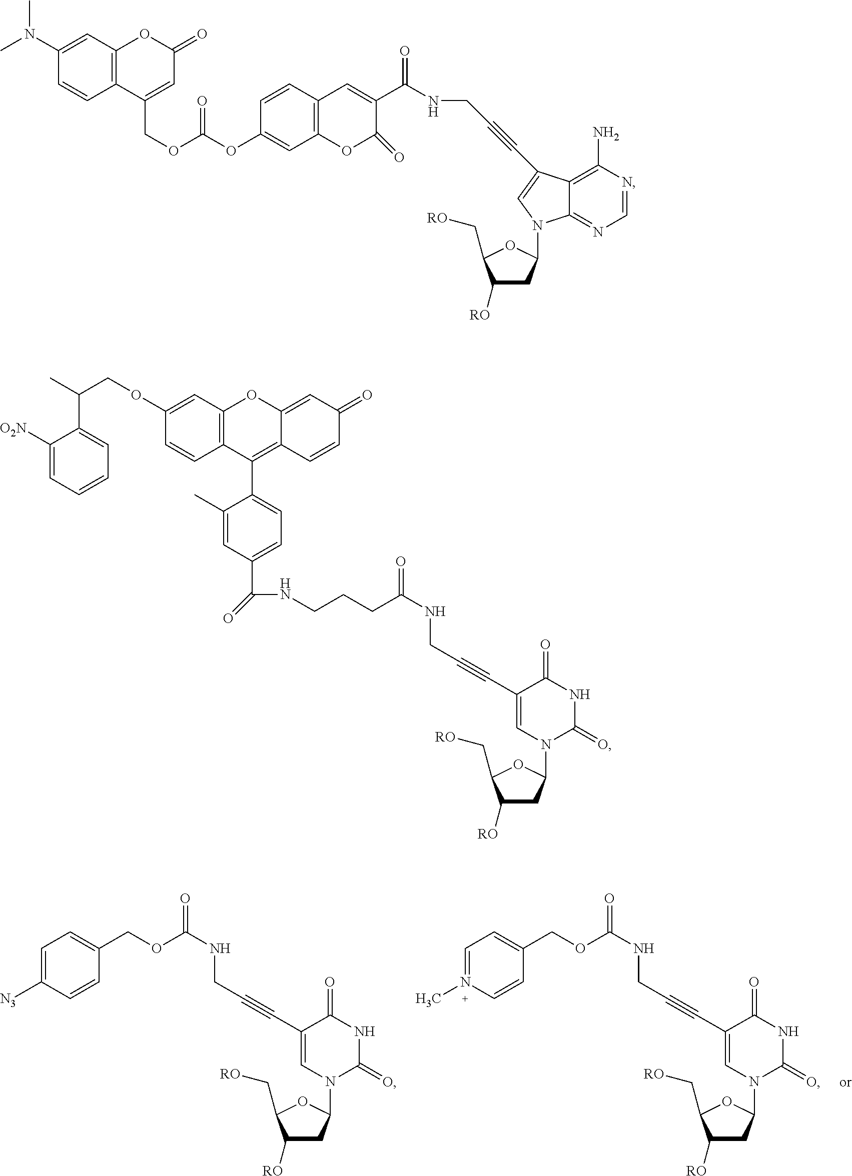

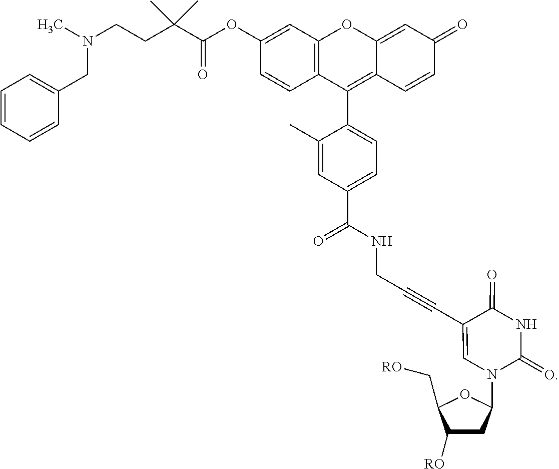

In some embodiments, all of the plurality of convertible residues in the nucleic acid polymer have the same structure.

In some embodiments, the plurality of convertible residues are capable of being converted by light of a wavelength of 325 nm, 360 nm, or 400 nm.

In some embodiments, the plurality of convertible residues are capable of being converted by light of a wavelength of between 400 nm to 850 nm.

In some embodiments, each of the plurality of convertible residues comprises a chemically modifiable moiety that is activatable by redox.

In some embodiments, the chemically modifiable moiety is capable of being activated by localized oxidation.

In some embodiments, the chemically modifiable moiety is capable of being activated by oxidation using electrodes.

In some embodiments, the first state and the second state of the plurality of convertible residues are readable by sequencing.

In some embodiments, the first state and the second state of the plurality of convertible residues are readable by nanopore sequencing.

In some embodiments, the first state and the second state of the plurality of convertible residues are readable by sequencing by synthesis.

In some embodiments, each of the plurality of convertible residues is capable of being independently and selectively converted.

In some embodiments, the nucleic acid polymer further comprises a plurality of spacer residues linked via the backbone of the nucleic acid polymer, wherein each of the plurality of convertible residues are separated by one or more spacer residues of the plurality of spacer residues.

In some embodiments, the iterative spacing among the plurality of convertible residues conforms to a resolution of a writing mechanism for encoding data on the nucleic acid polymer.

In some embodiments, the resolution of the writing mechanism is at least 1 nm.

In some embodiments, the plurality of spacer residues do not interfere with reading of the convertible residues.

In some embodiments, the plurality of spacer residues in the nucleic acid polymer are the same spacer residues.

In some embodiments, the plurality of spacer residues comprise two or more different types of spacer residues.

In some embodiments, the nucleic acid polymer consists essentially of spacer residues. In some embodiments, each of the plurality of convertible residues are separated by 2, 3, 4, 5, 6, 7, 8, 9, 10, 20, 30, 40, or 50 spacer residues.

In some embodiments, the plurality of spacer residues are naturally occurring nucleobases.

In some embodiments, the nucleic acid polymer further comprises one or more delimiters linked to the backbone of the nucleic acid polymer.

In some embodiments, each of the one or more delimiters comprises one or more naturally occurring nucleobases or non-naturally nucleobases.

In some embodiments, the one or more delimiters comprise naturally occurring nucleobases.

In some embodiments, the one or more delimiters separate two or more adjacent data fields within the nucleic acid polymer.

In some embodiments, the nucleic acid polymer further comprises one or more data tags.

In some embodiments, the one or more data tags comprise one or more naturally occurring nucleobases or non-naturally nucleobases.

In some embodiments, the one or more data tags are present at the 5′ or 3′ end of the nucleic acid polymer.

In some embodiments, the one or more data tags are incorporated to the nucleic acid polymer during synthesis of the nucleic acid polymer, during conversion of the plurality of convertible residues to the second state, or via ligation after the plurality of convertible residues are converted to the second state.

In another aspect, provided herein are systems for data writing, the system comprising: a writable nucleic acid polymer comprising a plurality of convertible residues iteratively spaced along and linked to the backbone of the nucleic acid polymer, wherein each of the plurality of convertible residues has a first state and is capable of being converted from the first state into a second state, the first state and the second state being different; wherein the plurality of convertible residues are covalently linked to the nucleic acid polymer in the first state and in the second state; and wherein the nucleic acid polymer comprises a sequence for priming a polymerase at the 3′ end of the nucleic acid polymer; and an enzyme conjugated with a sensitizer, wherein the sensitizer receives and transmits energy to the convertible residues.

In some embodiments, the system further comprises an energy source for providing light or redox energy. In some embodiments, the energy source provides light. In some embodiments, the energy source provides redox energy. In some embodiments, the transmission of energy from the sensitizer to the convertible residues converts the convertible residues from the first state to the second state. In some embodiments, the convertible residues are selected

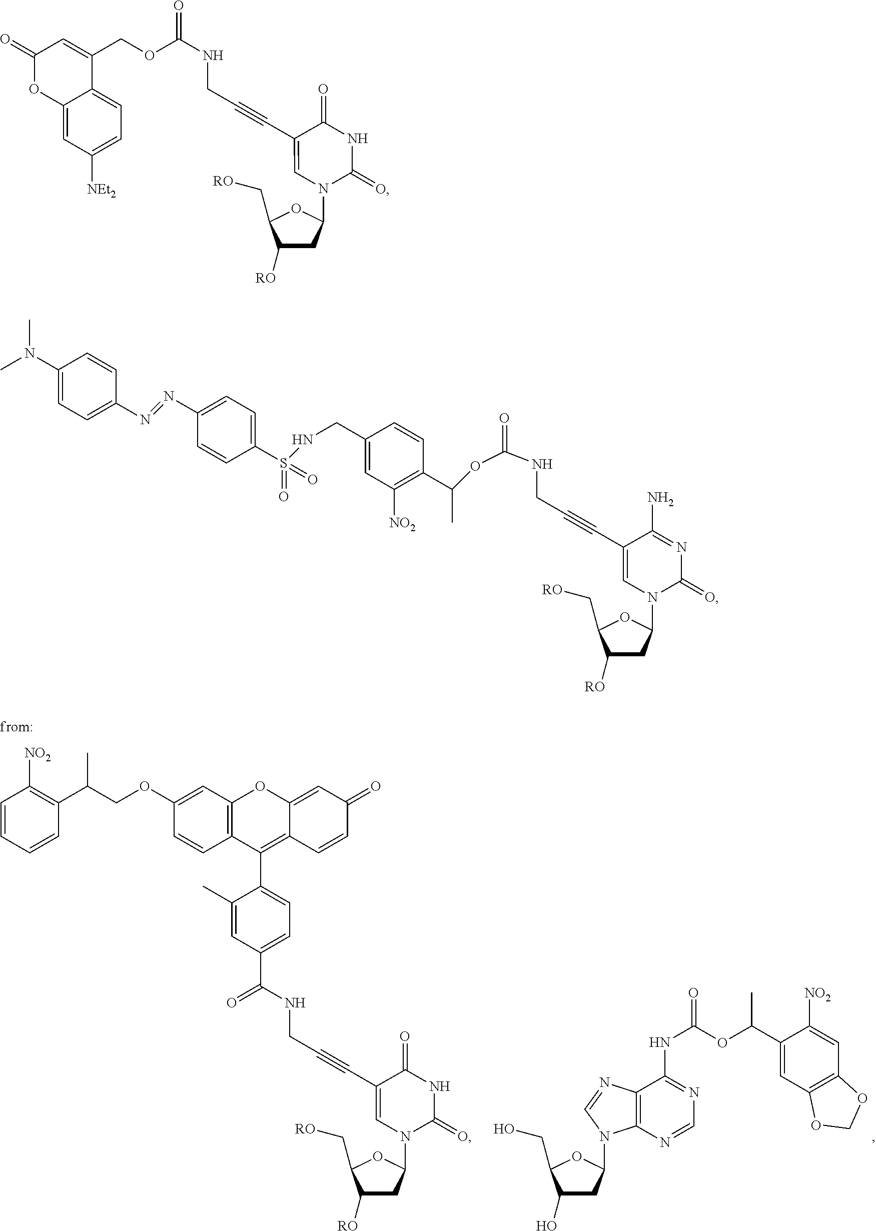

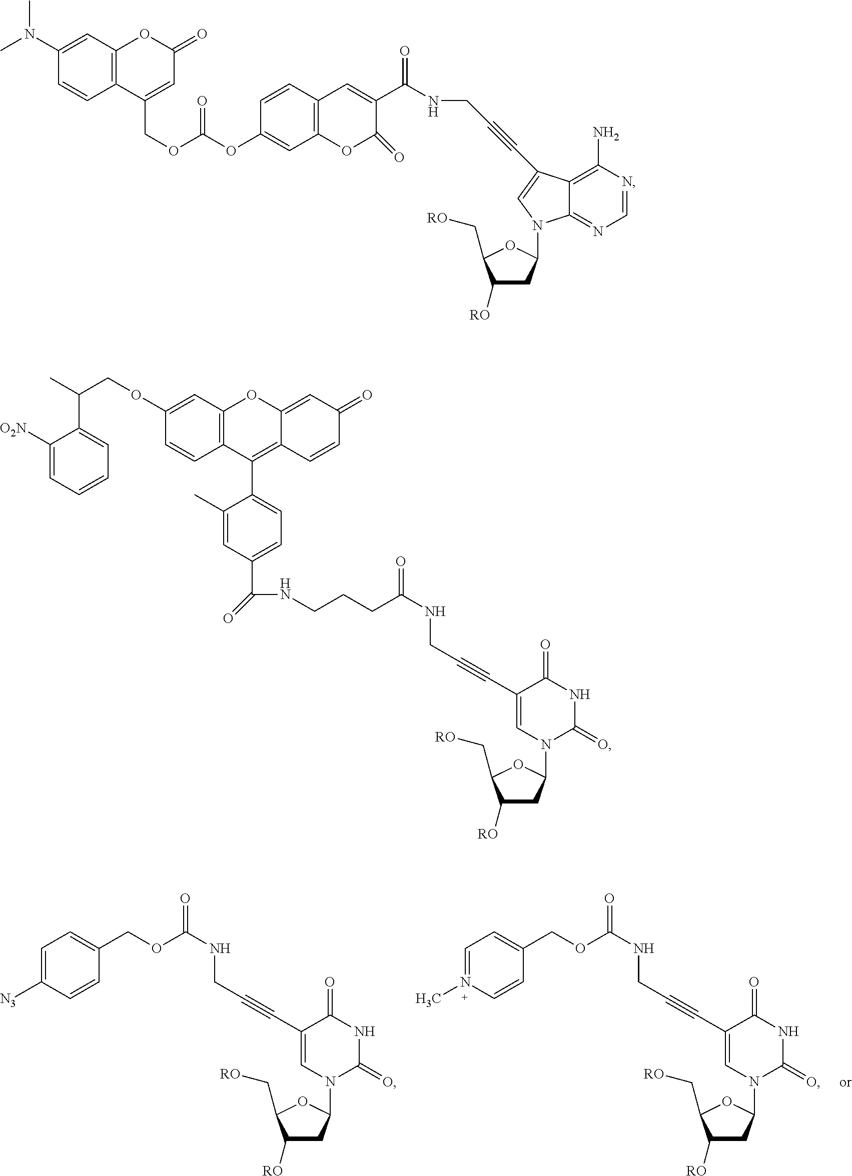

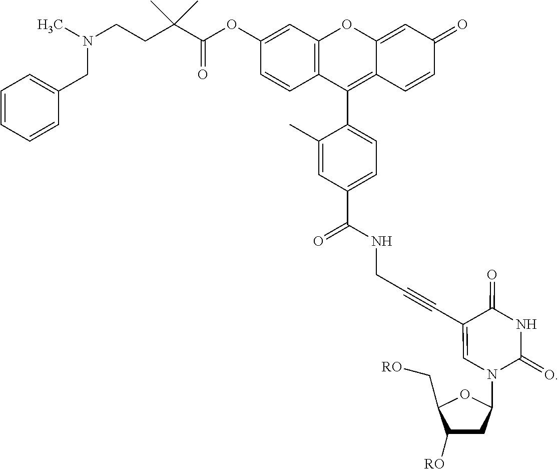

In some embodiments, the enzyme binds the writable nucleic acid polymer. In some embodiments, the enzyme is a polymerase. In some embodiments, the enzyme is a nucleic acid polymerase. In some embodiments, the enzyme is a template-dependent polymerase. In some embodiments, the enzyme is a template-independent polymerase. In some embodiments, the enzyme is a nuclease. In some embodiments, the enzyme is a helicase. In some embodiments, the enzyme is a nickase. In some embodiments, the sensitizer has a structure of:

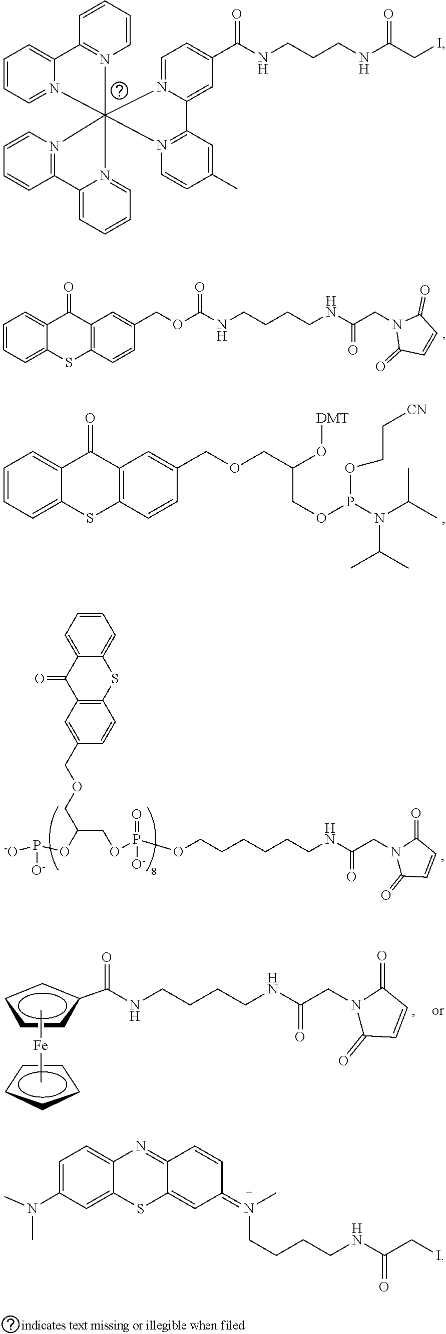

In some embodiments, the sensitizer is conjugated to the enzyme via a cysteine sidechain. In some embodiments, the system further comprises a primer oligomer, wherein the primer oligomer has a sequence complementary to the sequence at the 3′ end of the nucleic acid polymer. In some embodiments, the enzyme produces a nucleic polymer complementary to the writable nucleic acid polymer when writing data on to the writable nucleic acid polymer. In some embodiments, the system further comprises a set of triphosphate residues. In some embodiments, the triphosphate residues are dNTPs or NTPs. In some embodiments, the triphosphate residues are modifiable dNTPs or NTPs.

In yet another aspect, also provided herein are various methods for writing data onto a writable nucleic acid polymer, comprising: (a) providing in a solution a writable nucleic acid polymer that comprises a plurality of convertible residues iteratively spaced along and linked to the backbone of the nucleic acid polymer, wherein each of the plurality of convertible residues has a first state and is capable of being converted from the first state into a second state, the first state and the second state being different; wherein the plurality of convertible residues are covalently to the nucleic acid polymer in the first state and in the second state; and wherein the nucleic acid polymer comprises a sequence for priming a polymerase at the 3′ end of the nucleic acid polymer; and (b) providing an enzyme conjugated with a sensitizer that receives and transmits energy to the convertible residues; and (c) selectively converting one or more of the plurality of convertible residues into the second state such that a data encoded polymer is generated.

In some embodiments, the transmission of energy from the sensitizer to the convertible residues converts the convertible residues from the first state to the second state.

In some embodiments, the transmission of energy from the sensitizer to the convertible residues occurs as the enzyme moves along the writable nucleic acid polymer.

In some embodiments, the transmission of energy from the sensitizer to the convertible residues occurs when the enzyme is within proximity to the convertible residues of the writable nucleic acid polymer.

In some embodiments, one or more of the plurality of convertible residues are selectively converted into the second state by light pulses, voltage pulses, an enzymatic agent, or a redox potential.

In some embodiments, one or more of the plurality of convertible residues are selectively converted into the second state by light.

In some embodiments, one or more of the plurality of convertible residues are selectively converted into the second state by a redox potential. In some embodiments, the enzyme binds the writable nucleic acid polymer.

In some embodiments, the enzyme is a polymerase. In some embodiments, the enzyme is a nucleic acid polymerase.

In some embodiments, the convertible residues are:

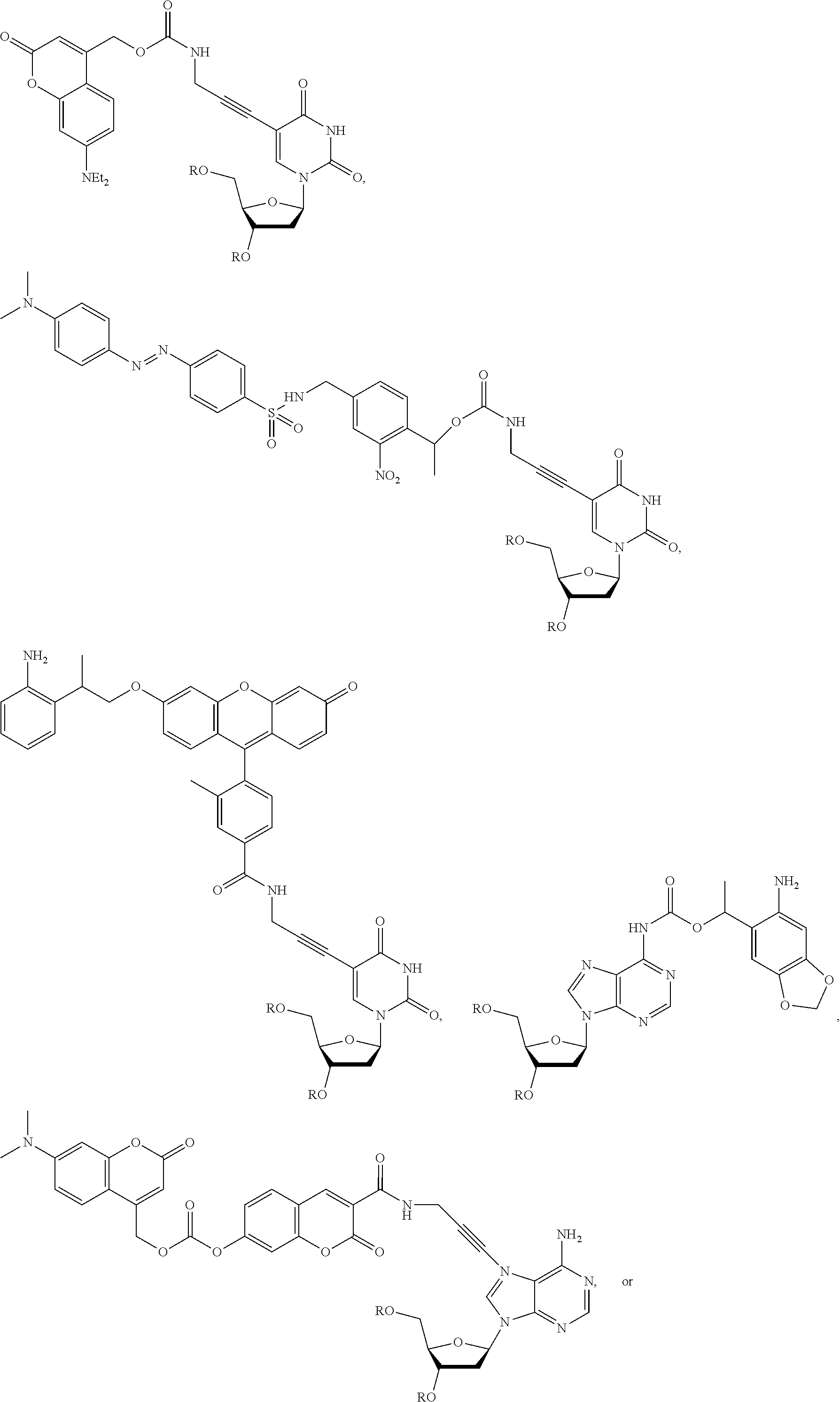

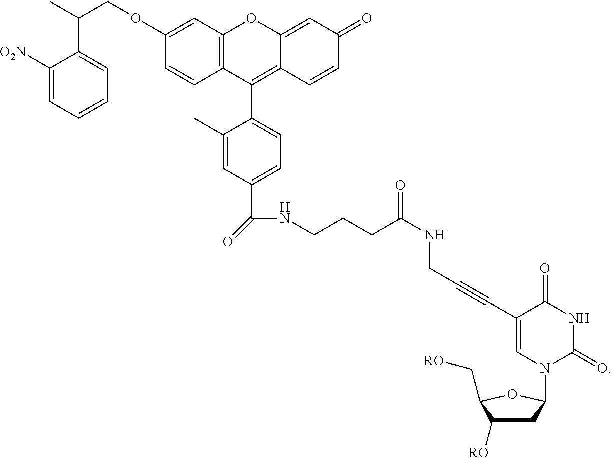

In some embodiments, the sensitizer has a structure of:

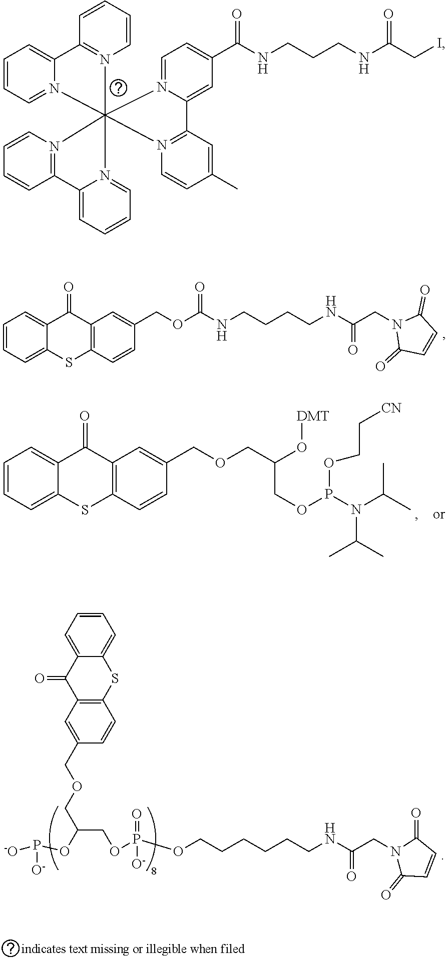

In some embodiments, the sensitizer is conjugated to the enzyme via a cysteine sidechain.

In some embodiments, the method further comprises: (c′) adding to the solution a primer oligomer, wherein the primer oligomer has a sequence complementary to the sequence at the 3′ end of the nucleic acid polymer.

In some embodiments, the method further comprises: (c″) adding to the solution a set of triphosphate residues, wherein the set of triphosphate residues comprises triphosphate residues having a first structure and triphosphate residues having a second structure.

In some embodiments, the set of triphosphate residues are added such that a final concentration of triphosphate residues having the first structure is lower than a final concentration of triphosphate residues having the second structure.

In some embodiments, the ratio of the triphosphate residues having the first structure to the triphosphate residues having the second structure results in the enzyme pausing as the enzyme moves along writable nucleic acid polymer and reaches residues of the template complementary to the triphosphate residues having the first structure.

In yet another aspect, further provided herein are nucleic acid polymerases for use in encoding data into a nucleic acid polymer, comprising: a nucleic acid polymerase conjugated with a sensitizer, wherein the sensitizer is a molecule capable of receiving and transmitting energy.

In some embodiments, the sensitizer is conjugated to the nucleic acid polymerase via cysteine side-chains. In some embodiments, the sensitizer is a molecule capable of receiving and transmitting light energy. In some embodiments, the sensitizer is a molecule capable of receiving and transmitting redox energy. In some embodiments, the sensitizer has a structure of:

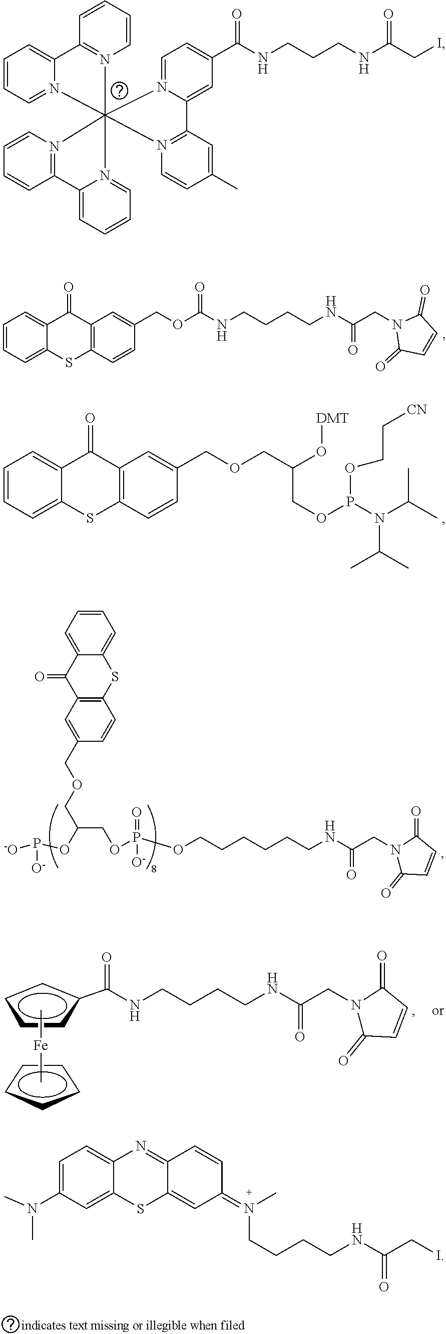

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIGS. 1A and 1B provide a schematic of a writable nucleic acid polymer in accordance with various embodiments.

FIG. 2 provides a schematic of generating a writable nucleic acid polymer utilizing polymerase extension via a rolling circle reaction in accordance with various embodiments.

FIG. 3 provides a schematic of generating a writable nucleic acid polymer utilizing chemical synthesis and ligation in accordance with various embodiments.

FIG. 4 provides a schematic of writing data using convertible residues (e.g., residues comprising chemically modifiable moieties) on a DNA strand utilizing a sensitizer conjugated to a polymerase enzyme in accordance with various embodiments.

FIGS. 5A to 5D provide molecular structure diagrams of various photoactive convertible residues for use in a writable nucleic acid polymer in accordance with various embodiments.

FIG. 6A provides molecular structure diagrams of various caged groups for use in a writable polymer in accordance with various embodiments.

FIG. 6B provides a schematic of dual-bit convertible residues for use in a writable nucleic acid polymer in accordance with various embodiments.

FIGS. 7A to 7E provide molecular structure diagrams of various sensitizer groups for performing chemical alteration in accordance with various embodiments.

FIG. 7F provides molecular structure diagrams of various redox active convertible residues for use in a writable nucleic acid polymer in accordance with various embodiments.

FIG. 8 provides molecular structure diagrams of various photocaging groups for use in a writable polymer in accordance with various embodiments.

### DETAILED DESCRIPTION

Provided herein are compositions of data-encodable polymers (e.g., nucleic acid polymers), and methods and systems thereof, for data encoding/decoding (e.g., writing/reading) and data storage. Also provided herein are method of making the polymers (e.g., nucleic acid polymers) described herein.

Turning now to the drawings and data, compositions and systems of nucleic acid data storage, methods of use and methods of synthesis, in accordance with various embodiments, are disclosed. In several embodiments, a system of data storage comprises writable nucleic acid polymers having a plurality of repeated convertible residues (e.g., chemically alterable group). In some embodiments, a system of data storage comprises a polymerase to promote chemical alteration of the convertible residue. In some embodiments, a system of data storage comprises a polymerase conjugated with a sensitizer group to promote chemical alteration of the convertible residue. Accordingly, a writable nucleic acid polymer is akin to a “blank tape” that does not initially store data but is encodable, wherein the writable nucleic acid polymer may be encoded by converting (e.g., chemically altering) one or more of the convertible residues utilizing the polymerase or sensitizer conjugated polymerase. In some embodiments, conversion (e.g., chemical alteration) of groups of the repeated convertible residues can be thought of as a binary code, where each convertible residue is akin to a “bit,” unaltered groups are akin to a “0” and groups that have been altered are akin to a “1.” It should be understood, however, that a binary code is not the only possibility, and codes can be written in ternary, quaternary, or other numeral system code, which can be done utilizing multiple types of convertible residues or performing multiple writings to further alter the state a convertible base. In some embodiments, the conversion of a convertible residue is stable, or permanent, which allows for long-term archiving. In some embodiments, the combination of two juxtaposed convertible residues comprises a “bit”. In embodiments utilizing two juxtaposed convertible residues, alteration of one group is akin to a “0” in the binary sense, and alteration of the other or both convertible residue is akin to a “1.”

In some embodiments, the terms “writable” and “data-encodable” are used herein interchangeably. In some embodiment, the terms “writing” and “data encoding” are used herein interchangeably.

In other embodiments of the systems provided herein, the systems comprise two or more sets of convertible residues (e.g., chemical residues having different structures, such having different chemically modifiable moieties), where residue conversion (e.g., cage group removal from the residue) can be thought of as a binary code, and each convertible residues (or sets of 2 or more convertible residues) is akin to a “bit” of data. In some embodiments, convertible residues are utilized to encode a bit, where conversion of a first residues structure (i.e., a first set of convertible residues) is akin to a “0,” and conversion of a second residues structure (i.e., a second set of convertible residues) of the pair is akin to a “1”, and data can be encoded by selective conversion of residues along the polymer (e.g., a nucleic acid polymer). In some embodiments, a pair of convertible residues are utilized to encode a bit, where conversion of one residues of the pair is akin to a “0,” and conversion of both residues of the pair is akin to a “1” and data can be encoded by convertible residue pair conversions along the polymer. It should be understood, however, that a binary code is not the only possibility, and codes can be written in ternary, quaternary, or other numeral system code, which can be done utilizing multiple types of convertible residues or performing multiple writings to further alter the state a convertible residue. In some embodiments, the conversion of a convertible residues is stable, or permanent, which allows for long-term archiving.

In some embodiments, the convertible residues are convertible nucleobases.

In some embodiment, the nucleic acid polymer is a single-stranded nucleic acid polymer or a double-stranded nucleic acid polymer. In some embodiment, the nucleic acid polymer is a single-stranded nucleic acid polymer. In some embodiment, the nucleic acid polymer is a double-stranded nucleic acid polymer.

Described herein are various embodiments directed towards compositions of writable nucleic acid polymers. Any appropriate nucleic acid polymer can be utilized, including (but not limited to) DNA, RNA, phosphorothiate DNA, enantio-DNA, glycerol nucleic acids (GNA), threose nucleic acids (TNA), 2′-fluoro-DNA, 2′-O-methyl RNA, and locked nucleic acids (LNA). A nucleic acid polymer may be single stranded or double stranded, and data writing can be performed utilizing a polymerase and a single stranded or double stranded template. In several embodiments, a writable nucleic acid polymer comprises a plurality of convertible residues (e.g., residues comprising a chemically modifiable moiety) that are covalently linked to a polymer (e.g., a nucleic acid polymer) backbone. In certain embodiments, convertible residues are spaced apart to provide spatial resolution such that each bit of one or more convertible residues can be independently and selectively altered in accordance with encoding. In some embodiments, spacer residues linked via the polymer backbone are utilized to provide spaces between the convertible residues. In some embodiments, spacer residues are unreactive to the writing mechanism. In various embodiments, a writable nucleic acid polymer can further include delimiters and/or data tags for labeling the data, each of which can be provided by a particular sequence of nucleobases.

In some embodiments, the terms “chemically alterable group” and “convertible residue” are used herein interchangeably.

In some embodiments, any appropriate nucleic acid polymer can be utilized, including (but not limited to) DNA, RNA, phosphorothioate DNA, glycerol nucleic acids (GNA), threose nucleic acids (TNA), locked nucleic acids (LNA), and combinations thereof.

In some embodiments, the plurality of convertible residues are capable of being incorporated into the nucleic acid polymer by an enzyme. In some embodiments, the plurality of convertible residues are capable of being incorporated into the nucleic acid polymer by an enzyme, where a sensitizer is conjugated to the enzyme. In some embodiments, the plurality of convertible residues are capable of being incorporated into a nucleic acid polymer by a polymerase, where a sensitizer is conjugated to a polymerase. In some embodiments, the plurality of convertible nucleotides are capable of being incorporated into the nucleic acid polymer by a polymerase.

In some embodiments, the plurality of convertible residues are non-naturally occurring nucleobases. In some embodiments, the plurality of convertible residues are modified naturally occurring nucleobases or derivatives of naturally occurring nucleobases.

In some embodiments, each of the plurality of convertible residues comprises a chemically modifiable moiety (e.g., modifiable fluorophore, releasable fluorophore, removable photocage, removable quencher, redox modifiable molecule, etc.). In some embodiments, each of the plurality of convertible residues the chemically modifiable moiety is directly attached to the base of the convertible residues. In some embodiments, each of the plurality of convertible residues the chemically modifiable moiety is attached to the base without a linker. In some embodiments, the plurality of convertible residues are covalently linked to the backbone of the nucleic acid via the sugar.

In some embodiments, the residues conversion (i.e., from the first state to the second state) is performed by removing one or more removal groups from the residues (e.g., nucleobases). In several embodiments, the removable group is a caging group.

In one embodiment, the chemically modifiable moiety is activatable by light, thereby converting from the first state into the second state. In some embodiments, the conversion from the first state into the second state occurs via an irreversible reaction. In some embodiments, the convertible residue becomes a naturally occurring nucleobase after conversion into the second state. In some embodiments, the convertible residue becomes a native nucleobase after conversion into the second state. In one embodiment, the convertible residue becomes guanine, adenine, thymine, or cytosine after conversion into the second state. In some embodiments, the backbone of the polymer (e.g., phosphate and sugar in nucleic acid polymer) remain unchanged during the conversion from the first state into the second state. In some embodiments, the chemically modifiable moiety is activatable by light, voltage, enzymatic agent, chemical reagent, or a redox agent, thereby converting from the first state into the second state. In some embodiments, the chemically modifiable moiety comprises one or more photo-removable or photo-cleavable groups.

In some embodiments, the convertible residue is selected from the group consisting of O6-guanine, N2-guanine, N7-guanine, N6-adenine, N5-adenine, O4-thymine, N3-thymine, 2-thio-thymine, 4-thio-thymine, N4-cytosine, or N3-cytosine.

In some embodiments, the first state and the second state of the plurality of convertible residues are readable by a sequencing method capable of detecting and differentiating non-naturally occurring and/or modified nucleobases. In some embodiments, the first state and the second state of the plurality of convertible residues are readable by nanopore sequencing. In some embodiments, the first state and the second state of the plurality of convertible residues are readable by sequencing by synthesis. In some embodiments, when the plurality of convertible residues are converted to the second state, properties of the plurality of convertible residues are modified (e.g., having reduced size, altered shape, modified H-bonding, and/or modified polymerase substrate ability) as compared to the first state. In some embodiments, one or more of the plurality of convertible residues are capable of being converted from the second state into a third state; wherein the one or more of the plurality of convertible residues are attached covalently to the nucleic acid polymer in the third state. In some embodiments, each of the plurality of convertible residues is capable of being independently and selectively converted.

In some embodiments, the polymers described herein (e.g., nucleic acid polymers) comprise two or more different sets of convertible residues, each set of convertible residues has a first state and is capable of being converted from the first state into a second state, the first state and the second state being different. In some embodiments, each of the plurality of convertible residues comprises a chemically modifiable moiety that can be activated by light, and the two or more different sets of convertible residues are activatable by light of a different wavelength. In some embodiments, a first set of convertible residues is activatable by light of a first wavelength, and a second set of convertible residues is activatable by light of a second wavelength, the first wavelength and the second wavelength being different.

In certain embodiments, convertible residues (or pairs of convertible bases) are iteratively spaced apart to provide spatial resolution such that each nucleobase (or each set or pair) can be independently and selectively converted in accordance with encoding. In some embodiments, iteratively spaced can be referred to as approximately regularly spaced. In certain embodiments, convertible residues are stochastically or irregularly spaced apart, but data is encoded by identifying and selectively converting nucleobases to yield an encoded polymer. In some of the embodiments utilizing stochastically or irregularly spaced convertible residues, the data encoding mechanism may skip any convertible residues as necessary until it reaches the right convertible residue in accordance with the code.

In several embodiments, a writing procedure is utilized to encode a writable nucleic acid with data. In some embodiments, data encoding can be performed by selectively altering convertible residues of a nucleic acid molecule such that the written nucleic acid molecule contains a sequence of unaltered and altered, akin to a binary code of “zeros” and “ones”. In some embodiments, when a bit comprises two convertible residues, data encoding can be performed by selectively altering one or both of the convertible residues of each bit to generate a code, which can be performed to generate a binary code. In many embodiments, a convertible residue is altered via light, voltage, enzymatic agent, chemical reagent, and/or a redox agent in conjunction with a sensitizer conjugated to a polymerase.

Various embodiments provided in this disclosure utilize a template-dependent DNA polymerase and a conjugated sensitizer to selectively encode modifications in nucleobases as the polymerase travels along the template. Unlike other template-independent enzymatic approaches that store data in DNA by its sequence, data can be stored in specific modifications of a DNA template. In many embodiments, the DNA modifications are designed to be switched structurally by light or electronic or voltage potential pulses that are imparted upon the DNA as it is being copied by the enzyme. The light or electronic pulses can be used to induce chemical changes in the DNA, and the sequence of these alterations can act as “bits” (e.g., binary bits of ones and zeros) of data. To assist in localizing the light or redox energy to nucleotides near the enzyme, in several embodiments, the polymerase is modified to contain one or more sensitizer groups that capture energy from light or electrons/holes (redox) and then transfer the energy to nearby reactive groups in the DNA template. Various embodiments are also directed to designs of DNA templates with groups that can be modified by light or redox to render local chemical or optical changes. As the polymerase proceeds along the template, making a copy in the presence of deoxyucleoside triphosphates (dNTPs), pulses of light or redox potential are applied in accordance with many embodiments, resulting in local chemical alterations one after the other along the strand. The sequence of structures and their alterations can encode digital data. At a subsequent time, this data can be “read” by sequencing methodologies, including nanopore sequencing by electric current changes (such as by Oxford Nanopore Technologies PromethION, MinION, and GridION sequencing platforms (Oxford, UK)), by optical sequencing using a plasmonic nanopore device, by shotgun sequencing methods, or Pacific Bioscience's Single Molecule, Real-Time (SMRT) sequencing platform (Menlo Park, CA). Also, a nanopore device can be fabricated or manufactured for reading the data. The nanopore can be comprised of solid-state materials or can contain one or more proteins.

In some embodiments, the data written (encoded) nucleic acid polymers are stored in accordance with standard nucleic acid storage protocols. For instance, data written nucleic acid polymers can be stored dry, as a precipitate, or in an appropriate nuclease-free solution at room temperature, or at colder temperatures (e.g., −20° C.). Stabilizers such as (for example) alcohol, chelating agents and nuclease inhibitors, may be included with the stored nucleic acid.

In several embodiments, template DNAs of repeating sequence comprising light- or redox-alterable groups (e.g., chemically modifiable moieties) are utilized. An advantage of a repeating sequence template is that it has predictable and tunable spacing of the alterable groups, enabling the user to control the proximity of the DNA polymerase to the alterable groups. In many embodiments, however, non-repeating sequence DNAs can be utilized, which can be obtained from biological sources and labeled later with alterable groups. An advantage of non-repeating DNAs is that DNAs from biological sources can have very long lengths and can contain a unique sequence at the 3′ end that can serve as a unique primer binding site for the sensitized polymerase.

In addition to storing data in DNA, various methods disclosed herein may also be used for other applications, including local labeling of DNA, control of DNA folding and formation of nanostructures, control of protein interactions with DNA, and alteration of methylation patterns.

In some embodiments, the use of solid supports to sequester and stabilize the nucleic acid such as polymer beads, glass beads, or mineral solids are also contemplated. In some embodiments, the data on the written (encoded) nucleic acid polymers is decoded or read by sequencing by synthesis (SBS). And in some embodiments, a sequencer capable of reading modified and/or unmodified nucleobases can be utilized to decode or read data, such as Oxford Nanopore Technologies PromethION, MinION, and GridION sequencing platforms (Oxford, UK) or Pacific Bioscience's Single Molecule, Real-Time (SMRT) sequencing platform (Menlo Park, CA).

The present disclosure overcomes many of the limitations associated with traditional nucleic acid data storage by separating the synthesis and data encoding into distinct steps. The disclosure provides molecular strategies for producing long strands of writable nucleic acids that, in themselves, do not encode data, but rather provide a template with the capacity for being written. Writable nucleic acid polymers can be produced in bulk in advance of data encoding. The disclosure further provides compositions and systems comprising convertible residues (and pairs of convertible residues) that act as “bits” of data, which can be switched from a first state into a second state, thus defining “0” and “1” in binary code. The disclosure further provides methods for writing data into the writable nucleic acid polymers provided herein at the single molecule level, thus consuming negligible amounts of material. Data writing may be achieved chemically or physically, utilizing (for example) light pulses or voltage pulses. Finally, because the written nucleic acid polymers are long, they encode more data per molecule than do short DNAs and can be efficiently and rapidly read by various sequencers existing within the current market. The compositions, systems, and methods described herein greatly increase the speed and density of nucleic acid data encoding while lowering cost.

### Writable Polymers for Encoding Data

In one aspect, provided herein are nucleic acid polymers for encoding data, comprising a plurality of convertible residues, iteratively spaced along and covalently linked to the backbone of the polymer, wherein each of the plurality of convertible residues has a first state and a is capable of being converted from the first state into a second state, and wherein the plurality of convertible residues are covalently linked to the polymer in the first state and in the second state. In some embodiments, the first state and the second state are different (e.g., the convertible residues have different structures when in the first and the second state). In some embodiments, the plurality of convertible residues in the first state and in the second state are readably by polymerase. In some embodiments, the plurality of convertible residues in the first state and in the second state are readable by polymerase, where a sensitizer group is conjugated to the polymerase to promote local chemical alteration of the linked convertible residues (e.g., different structures). In some embodiments, the plurality of convertible residues are repeatedly spaced along the backbone of the polymer being copied by the enzyme. In some embodiments, the plurality of convertible residues are incorporated into the polymer the polymerase as it copies an existing DNA strand.

In some embodiments, the polymers described herein are nucleic acid polymers and the plurality of convertible residues are convertible residues.

In certain embodiments, the convertible residues are iteratively spaced apart to provide spatial resolution such that each residue can be independently converted. In some embodiments, any appropriate spacer (e.g., non-writable, i.e., unreactive to the data writing mechanism) are between the convertible residues. In some embodiments, residues linked by the polymer backbone can be utilized as spacers. In some embodiments, the spacers spaced between the convertible residues in accordance with the spatial resolution of the writing mechanism and/or writing device. In some embodiments, spacers are residues, which may be unreactive to the writing mechanism. In various embodiments, the polymer further comprises delimiters and/or data tags for labeling the data.

In some embodiments, the polymers described herein (e.g., nucleic acid polymers) further comprise a plurality of spacer residues linked via the backbone of the polymer, wherein each of the plurality of convertible residues are separated by one or more spacer residues of the plurality of spacer residues. In some embodiments, wherein the iterative spacing among the plurality of convertible residues conforms to a resolution of a writing mechanism for encoding data on the polymer. In some embodiments, the iterative spacing among two adjacent convertible residues is equal to or greater than a resolution of a data encoding mechanism for encoding data into the polymer. In some embodiments, the resolution of the writing mechanism is at least 1 nm. In some embodiments, the plurality of spacer residues do not interfere with reading of the convertible residues. In some embodiments, the plurality of spacer residues in the polymer are the same spacer residues. In some embodiments, the plurality of spacer residues comprise two or more different spacer residues (e.g., different nucleobases such as different naturally occurring nucleobases).

In some embodiments, the polymers described herein (e.g., nucleic acid polymers) consist essentially of spacer residues.

In some embodiments, each of the plurality of convertible residues are separated by 2, 3, 4, 5, 6, 7, 8, 9, 10, 20, 30, 40, or 50 spacer residues. In some embodiments, each of the plurality of convertible residues are separated by 6 spacer residues. In some embodiments, the plurality of spacer residues are naturally occurring nucleobases, non-naturally nucleobases, tetrahydrofuran abasic residues, or ethylene glycol residues, the plurality of spacer residues are naturally occurring nucleobases.

In some embodiments, the polymers described herein (e.g., nucleic acid polymers) further comprise one or more delimiters linked to the backbone of the polymer. In some embodiments, each of the one or more delimiters comprises one or more naturally occurring nucleobases or non-naturally nucleobases. In some embodiments, the one or more delimiters comprise naturally occurring nucleobases. In some embodiments, the one or more delimiters separate two or more adjacent data fields within the polymer.

In some embodiments, the polymers described herein (e.g., nucleic acid polymers) further comprise one or more data tags. In some embodiments, the one or more data tags comprise one or more naturally occurring nucleobases or non-naturally nucleobases. In some embodiments, the polymer is a nucleic acid polymer, and the one or more data tags are present at the 5′ or 3′ end of the nucleic acid polymer. In some embodiments, the one or more data tags are incorporated to the nucleic acid polymer during the nucleic acid polymer is synthesized, during the plurality of convertible residues are converted to the second state, or via ligation after the plurality of convertible residues are converted to the second state.

In some embodiments, the polymer can have any number or length of monomeric units, for example, from as short as 10 monomeric units to longer than 100,000 monomeric units. In various embodiments, the polymer has greater than 500 monomeric units, greater than 1,000 monomeric units, greater than 5000 monomeric units, greater than 10,000 monomeric units, greater than 50,000 monomeric units, or greater than 100,000 monomeric units.

In some embodiments, the nucleic acid polymer comprises greater than 10 convertible residues. In some embodiments, the nucleic acid polymer comprises greater than 100 convertible residues. In some embodiments, the nucleic acid polymer comprises greater than 500 convertible residues. In some embodiments, the nucleic acid polymer comprises greater than 1,000 convertible residues. In some embodiments, the nucleic acid polymer comprises greater than 10,000 convertible residues. In some embodiments, the nucleic acid polymer comprises greater than 100,000 convertible residues.

In some embodiments, the ratio of the total number of monomeric units (e.g., nucleotides) to the convertible residues (e.g., convertible nucleobases) in the polymer (e.g., nucleic acid polymer) is between 2 to 100. In some embodiments, the ratio of the total number of monomeric units (e.g., nucleotides) to the convertible residues in the polymer (e.g., nucleic acid polymer) is between 2 to 10. In some embodiments, the ratio of the total number of monomeric units (e.g., nucleotides) to the convertible residues in the polymer (e.g., nucleic acid polymer) is between 10 to 50.

In some embodiments, the ratio of the total number of monomeric units (e.g., nucleotides) to the convertible residues (e.g., convertible nucleobases) in the polymer (e.g., nucleic acid polymer) is between 10 to 100. In some embodiments, the ratio of the total number of monomeric units (e.g., nucleotides) to the convertible residues (e.g., convertible nucleobases) in the polymer (e.g., nucleic acid polymer) is between 20 to 100. In some embodiments, the ratio of the total number of monomeric units (e.g., nucleotides) to the convertible residues (e.g., convertible nucleobases) in the polymer (e.g., nucleic acid polymer) is between 20 to 50. In some embodiments, the ratio of the total number of monomeric units (e.g., nucleotides) to the convertible residues (e.g., convertible nucleobases) in the polymer (e.g., nucleic acid polymer) is greater than 100.

### Writable Nucleic Acid Polymers

In certain embodiments, the polymers described herein (e.g., writable polymers) are nucleic acid polymers and the plurality of convertible residues are convertible residues. In certain embodiments, the polymers described herein are nucleic acid polymers comprising a plurality of convertible residues iteratively spaced along and covalently linked to the backbone of the nucleic acid polymer, wherein each of the plurality of convertible residues has a first state (e.g., having a first state structure) and is capable of being converted from the first state into a second state (e.g., having a second state structure), the plurality of convertible residues are covalently linked to the nucleic acid polymer in the first state and in the second state. In some embodiments, the first state and the second state are different and are both readable by polymerase. In some embodiments, the first state and the second state are different and are both readable by polymerase, where a sensitizer group is conjugated to the polymerase to promote local chemical alteration of the linked convertible residues (e.g., chemically alterable group or structures that vary from the first state to the second state). In some embodiments, the nucleobase in the second state is a natural nucleobase. In some embodiments, the nucleobase in the second state is scarless (i.e., in native form of nucleobase, such as guanine, adenine, thymine, or cytosine.

In some embodiments, the unwritten state is also referred to as the unconverted state, and the written state is also referred to the converted state.

Compounds in accordance with embodiments of the disclosure are based on nucleic acids having a plurality of convertible residues that are repeated along the polymer, which are akin to data bits. Each convertible residue can exist in two or more states, an unaltered state and at least a first altered state, in which the collection of altered states of the convertible residues denotes a data code (e.g., binary code). In several embodiments, writable nucleic acid polymers are synthesized with a plurality of convertible residues in an “unwritten” state that are capable of being converted. In some embodiments, two different convertible residues are employed as a pair for encoding a single bit: conversion of one encodes a “0” while conversion of the other or both encodes a “1”. These writable nucleic acids can be created having long lengths (e.g., 5 to 50 kb, or more) and can be produced in bulk, prior to data writing.

In some embodiments, a single convertible residue (e.g., chemically alterable group or convertible nucleotide) is utilized to encode a bit of data. In some embodiments, a set of two or more convertible residues is utilized to enable the encoding of a bit of data. In some embodiments, a pair of two different convertible residues are employed as a pair for enabling the encoding of a single bit. In some embodiments utilizing a pair of two different convertible residues, conversion of a first residues encodes a “0” while conversion of the other residue encodes a “1”. In some embodiments utilizing a pair of two different convertible residues, conversion of one residue encodes a “0” while conversion of both of the residues encodes a “1”.

In several embodiments, a writable nucleic acid polymer comprises a plurality of convertible residue (e.g., chemically alterable groups) attached to residues that are linked by the polymer backbone. In certain embodiments, bits of convertible residues are iteratively spaced apart to provide spatial resolution such that each group can be independently altered. The spatial resolution depends, at least in part, on the polymerase with conjugated sensitizer. Spatial resolution can be assessed and optimized through experimentation. Distance effects of photosensitization have been described in literature (see, e.g., K. A. Ryu, et al., Nat Rev Chem. 2021:5:322-337; and M. Klausen, et al., Chempluschem. 201:84:589-598: the disclosures of which are each incorporated herein by reference). Any appropriate spacer between the convertible residues can be utilized. In some embodiments, residues without attached convertible residues can be utilized as spacers. Because the distances between nucleobases in a double-stranded DNA polymer is about 0.34 nm, in accordance with numerous embodiments, three spacers are utilized for each nanometer of spatial resolution of the alteration-inducing source. In some embodiments, spacers are nucleobases, which may be unreactive to the writing mechanism. In various embodiments, a writable nucleic acid polymer can further include delimiters and/or data tags for labeling the data, each of which can be provided by a particular sequence of residues.

In several embodiments, a data encodable nucleic acid polymer comprises a plurality of convertible residues (e.g., convertible nucleobase or chemically alterable group) that are linked by the polymer backbone. In certain embodiments, convertible residues are stochastically or irregularly spaced apart, but data is encoded by identifying and selectively converting nucleobases to yield an encoded polymer. In some of the embodiments utilizing stochastically or irregularly spaced convertible residues, the data encoding mechanism may skip any convertible residues as necessary until it reaches the right convertible residue in accordance with the code. In certain embodiments, convertible residues (or sets of convertible residues or convertible nucleobases) are iteratively spaced apart to provide spatial resolution such that each convertible residues (or each set of convertible residue) can be independently converted. The spatial resolution depends, at least in part, on the writing mechanism. For instance, if an optical light source and device with 1 nm of resolution is used to alter nucleobases, then each convertible residues (or each set of residues) needs to be separated by at least 1 nm. Any appropriate spacer between the convertible residues (or each set of residues) can be utilized. In some embodiments, residues linked by the polymer backbone can be utilized as spacers. Because the distances between residues in a double-stranded DNA polymer is about 0.34 nm, in accordance with numerous embodiments, three spacers are utilized for each nanometer of spatial resolution of the alteration-inducing source. In some embodiments, spacers are residues (e.g., nucleobases), which may be unreactive to the writing mechanism. In various embodiments, a data encodable nucleic acid polymer can further include delimiters and/or data tags for labeling the data, each of which can be provided by a particular sequence of residues.

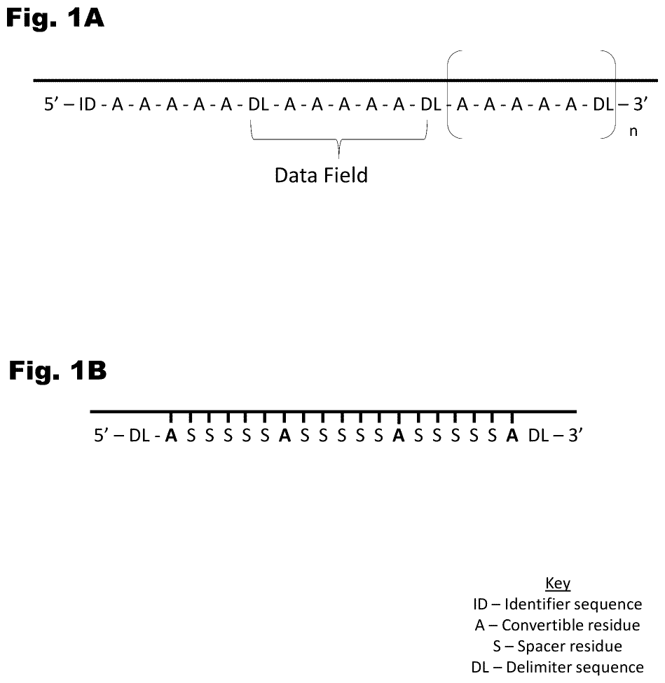

FIG. 1A illustrates an example of a writable nucleic acid polymer having a plurality of convertible residues (e.g., chemically alterable groups, or “A” as depicted in FIG. 1A). The writable nucleic acid polymer (e.g., nucleic acid polymer comprising convertible residues) comprises a repeating strand sequence, which can exist as a single-stranded or double-stranded molecule. Notably, because writing can be performed utilizing a polymerase conjugated with a sensitizer, separation of the two strands of the double stranded molecule may need to be performed prior to polymerase-mediated writing, depending on the polymerase and reaction conditions. In some embodiments, the repeating unit comprises convertible residues attached to a residue. In some embodiments, the repeating unit comprises convertible residues (e.g., chemically alterable groups or convertible nucleobases), which may be natural or unnatural, that can undergo chemical changes from a first structure state to a second structure state, akin to a switch from a “0” state to a “1” state. In some embodiments, the same residue is utilized to attach convertible residues. Thus, the convertible residue group is repeated in the nucleic acid polymer sequence in accordance with an appropriate spatial resolution. Prior to any data writing, convertible residues are initially provided in the unaltered state. In some embodiments, the repeating unit of the writable nucleic acid polymer comprises data fields that include a plurality of convertible residue, and may also contain spacers (e.g., “S” as depicted in FIG. 1B) or sequences that delimit or separate bits. FIG. 1B provides an exemplary concept of a data field sequence having a plurality of convertible residues separated by spacers. As shown, three spacers are utilized between each convertible residue which would provide the appropriate spatial resolution. It is understood that longer spacer sequences can be used in cases of lower bit-writing resolution. In some embodiments, a writable nucleic acid polymer includes one or more unique data tag sequences, denoting documentation such as type of data, date, or other information. A unique data tag sequence may be written during the synthesis of the writable DNA, or may be written during the data writing process, or may be added on to an end via a primer or may be added to the data strand via ligation or polymerase extension after data writing.

In various embodiments, writable nucleic acid polymers can be any length, for example, from as short as 15 nucleotides to longer than 100 kilobases. In various embodiments, a writable nucleic acid polymer is greater than 500 nucleotides long, is greater than 1000 nucleotides, is greater than 5000 nucleotides, is greater than 10,000 nucleotides, is greater than 50,000 nucleotides, or is greater than 100,000 nucleotides. Maximum lengths are only limited by the stability of the DNA, by the method used to make them, and by the method used to read the written data. Longer strands have the advantage of containing more data per molecule. Notably, current sequencing technologies can handle nucleic acid strands of tens to hundreds of thousands of bases in length (see N Kono and K. Arakawa, Dev Growth Differ. 2019:61:316-326; and Q Chen and Z. Liu, Sensors (Basel). 2019; 19:1886; the disclosures of which are each incorporated herein by reference).

Several embodiments are directed to convertible residues (e.g., chemically alterable groups), which can be attached to residues and incorporated into a writable nucleic acid polymer. A convertible residue, in accordance with various embodiments, is a group that is capable of being converted from a first chemical state into a second chemical state by a controlled reaction chemistry. Any appropriate mechanism to convert a convertible residue from a first state into a second state can be utilized, including (but not limited to) light pulses, voltage, enzymatic agent, chemical reagent, and/or redox pulses. It is understood that residues are not limited to naturally occurring nucleobase or nucleotide structures, but may also embody unnatural nucleobases or nucleotides, such as designer nucleobases.

In some embodiments, the structural change results in a conversion of a non-natural nucleobase (e.g., nucleobase in the first structural state) to a natural or native nucleobase (e.g., nucleobase in the second structure state). In some embodiments, the nucleobase in the second state is a natural nucleobase. In some embodiments, the nucleobase in the second state has no scar. In some embodiments, the nucleobase in the first state comprises a chemically modifiable moiety (e.g., removable photocage, removable quencher, releasable fluorophore, molecule capable of undergoing structural change due to oxidation or reduction). In some embodiments, the nucleobase in the first state does not comprise a linker (or a linker moiety) between the base of the nucleobase and the chemically modifiable moiety. In some embodiments, when the nucleobase in the first state is converted to the second state, the chemically modifiable moiety is removed and cleaved, thereby leaving the nucleobase in the second state a natural or native nucleobase. In some embodiments, when the nucleobase in the first state and in the second state are readable or recognizable by polymerase. In some embodiments, when the nucleobase in the first state and in the second state are readable or recognizable by polymerase, where a sensitizer group is conjugated to the polymerase to promote local chemical alteration of the linked convertible residues (e.g., chemically alterable groups or convertible nucleobase that may vary from the first state to the second state). In some embodiments, when the written nucleic acid polymer is readable by various sequencing methods, e.g., sequencing by synthesis (SBS).

Numerous embodiments are also directed to a writable nucleic acid polymer further incorporating one or more of spacers (e.g., “S” as depicted in FIG. 1B), delimiters (e.g., “DL” as depicted in FIGS. 1A and 1B), and data tags. In accordance with various embodiments, a spacer is molecular residue incorporated within a writable nucleic acid polymer that provides a requisite space between convertible residue (e.g., chemically alterable groups) in accordance with spatial resolution of the data writing sensitizer conjugated to the polymerase. In many embodiments, a spacer will be distinguishable from a convertible residue such that when the data is read in a sequencer, the spacer does not interfere with the ability to read the altered groups. In some embodiments, a spacer is unreactive with the data writing mechanism. In some embodiments, a writable nucleic acid polymer will utilize the same residue repeatedly for each spacer. In some embodiments, however, a writable nucleic acid polymer will utilize two or more different residues as spacers. Any appropriate residue that is distinguishable from the convertible residues may be utilized as spacers, including naturally occurring nucleobases, unnatural nucleobases, tetrahydrofuran abasic residues, and/or ethylene glycol residues.

In some embodiments, a spacer is distinguishable from convertible residues and/or converted nucleobases such that when the data is read in a sequencer, the spacer does not interfere with the ability to encode data and decode/read the encoded data. In some embodiments, a spacer is unreactive with the data encoding mechanism.

A delimiter, in accordance with various embodiments, is a residue that signifies a boundary. In some embodiments, a delimiter is utilized to separate two adjacent data fields. Any appropriate residue that is distinguishable from the convertible residues (e.g., chemically alterable groups) may be utilized as a delimiter, including naturally occurring nucleobases, unnatural nucleobases, tetrahydrofuran abasic residues, and/or ethylene glycol residues.

In another aspect, also provided herein are methods for generating a writable nucleic acid polymer, comprising providing a circular single-stranded oligonucleotide template, wherein the circular single-stranded oligonucleotide template is complementary to a repeating data field that comprises convertible residues; and incubating the circular single-stranded oligonucleotide template in the presence of a nucleic acid primer, a polymerase, and triphosphate nucleotides, wherein the triphosphate nucleotides comprise convertible residues in a first state and are capable of being converted from the first state into a second state, the first state and the second state being different.

In some embodiments, the circular single-stranded oligonucleotide template comprises nucleobases complementary to the convertible residues, and wherein the complementary nucleobases are iteratively spaced such that the incubation of the template with the nucleic acid primer, the polymerase, and the triphosphate nucleotides provides a nucleic acid polymer comprising a plurality of the convertible residues iteratively spaced along and covalently linked via the backbone of the nucleic acid polymer: wherein the plurality of the convertible residues are covalently linked to the nucleic acid polymer in the first state and in the second state. In such embodiments, the polymerase may be conjugated to a sensitizer as described herein.

In some embodiments, the repeating data field further comprises spacer nucleobases, and wherein the triphosphate nucleotides further comprise triphosphate spacer nucleotides.

In another aspect, also provided herein are methods for generating a writable nucleic acid polymer, comprising chemically synthesizing a plurality of oligomers, each oligomer comprises a plurality of convertible residues iteratively spaced along and linked via the nucleic acid polymer backbone, wherein each of the plurality of convertible residues has a first state and is capable of being converted from the first state into a second state: wherein the plurality of convertible residues are attached covalently to the nucleic acid polymer in the first state and in the second state, the first state and the second state being different; and ligating the plurality of oligomers to form the writable nucleic acid polymer.

In some embodiments, each of the plurality of oligomers comprises a plurality of spacer residues linked via the backbone of the nucleic acid polymer, wherein each of the plurality of the convertible residues is separated by one or more spacer residues of the plurality of spacer residues. In some embodiments, the ligating step is via chemical ligation. In some embodiments, the ligating step is via enzymatic ligation. In some embodiments, a complementary DNA splint is used in the ligating step.

In some embodiments, the method further comprising annealing a plurality of complements to the oligomers prior to the ligating step.

In several embodiments, a data tag is a string of residues (typically 4 or more residues) that signifies certain data. For instance, a data tag can signify type of data, date, data source, or any other information. Any appropriate residues that are distinguishable from the convertible residues (e.g., chemically alterable groups) may be utilized as data tag residues, including naturally occurring nucleobases, unnatural nucleobases, tetrahydrofuran abasic residues, and/or ethylene glycol residues.

Writable nucleic acids can be generated by any appropriate method for generating long nucleic acid polymers. Generally, in accordance with various embodiments, polymerase extension or chemical synthesis is utilized to generate writable nucleic acid polymers. If polymerase extension is utilized, appropriate residues that can be polymerized by the polymerase are to be utilized. In such aspects, a sensitizer may be conjugated to the polymerase. If chemical synthesis is utilized, a broader range of residues are available for incorporation, but generally synthesis results in shorter nucleic acid strands (e.g., between 10 and 200 residues), which can be ligated together to generate longer nucleic acid polymers. It is understood that both polymerase and ligation methods can construct repeating writable polymers in either single-stranded or double-stranded states. In some embodiments, a chemically modifiable moiety is attached to a residue (e.g., a convertible residue monomer) prior to incorporation into the polymer. In some embodiments, a convertible residue (e.g., chemically alterable group) is attached to a set of repeated convertible residues or other residues of the polymer after it is synthesized.

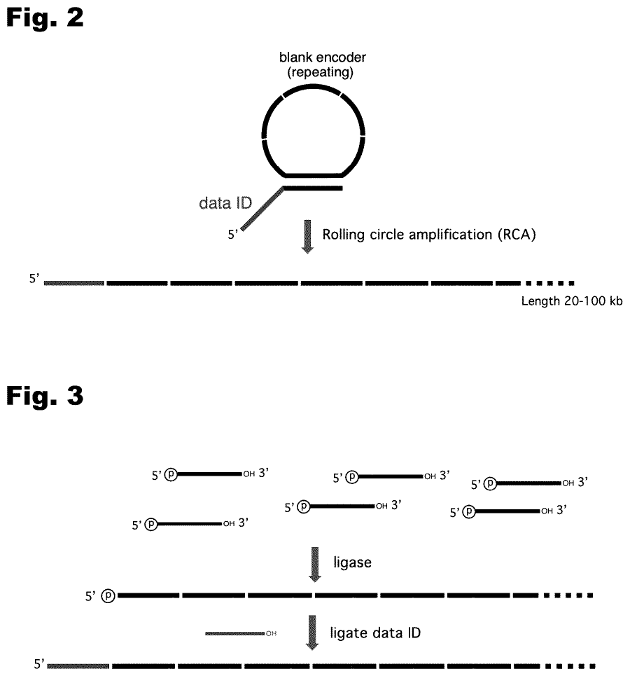

Provided in FIG. 2 is an example of generating a writable nucleic acid utilizing polymerase extension, and in particular, the figure illustrates an enzymatic rolling circle reaction method. In certain embodiments, a circular single-stranded DNA oligonucleotide is utilized as template (M. G. Mohsen and E. T. Kool, Acc Chem Res. 2016; 49; 2540-2550, the disclosure of which is incorporated herein by reference). The circular single-stranded DNA oligonucleotide is complementary to the repeating data field that comprises residues with attached (or for attaching) convertible residues. In various embodiments, the circular single-stranded DNA oligonucleotide further comprises spacers, delimiters, and/or data tags. In various embodiments, the circular DNA size is 20-10.000 nucleotides in length, preferably 20-200 nucleotides in length, and more preferably 45-95 nucleotides in length.

Once the nucleic acid circle template encoding the repeating data fields is constructed, it is incubated with a nucleic acid primer, a polymerase, a suitable buffer to support polymerase activity, and nucleoside triphosphates suitable for generating the nucleic acid polymer. The primer binds the circle, and the polymerase then produces a long repeating complement of the circle. Rolling circle nucleic acid synthesis is documented to proceed for many thousands of nucleotides, producing long DNA repeats (see M. M. Ali, et al., Chem Soc Rev. 2014:43:3324-41; and M. G. Mohsen and E. T. Kool. Acc Chem Res. 2016 Nov. 15; 49 (11): 2540-2550; the disclosures of which are incorporated herein by reference). In some embodiments, a data tag is utilized, which may be included at the remote 5′-end of the primer and remains non-complementary to the DNA circle. Rolling circle DNA synthesis in this case will result in the repeating nucleic acid polymer with a data tag attached to the 5′-end. If writable nucleic acid polymers are desired to be double-stranded, a primer complementary to the repeating data fields can be used together with a polymerase and nucleotides complementary to the first polymer to generate the complementary strand.

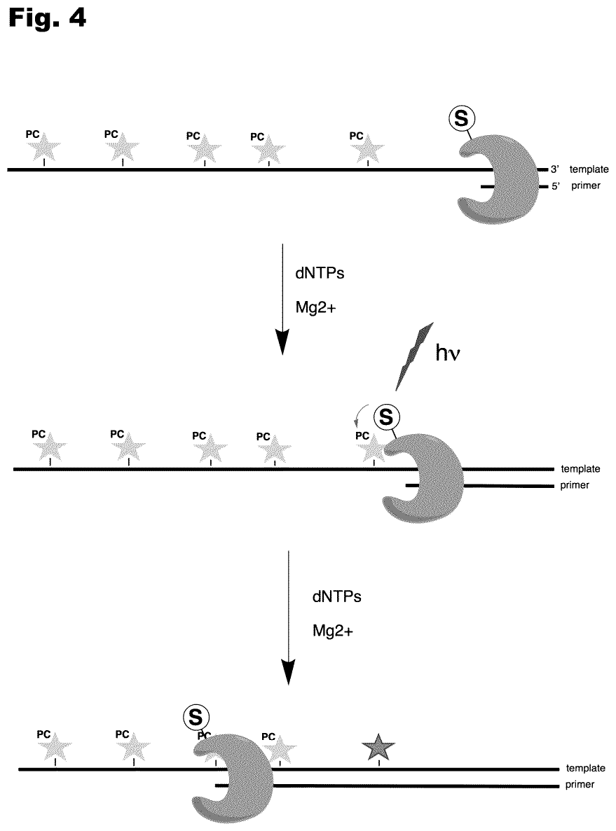

FIG. 3 illustrates a chemical synthesis and ligation method for generating a writable nucleic acid. In some instances, nucleotides for incorporation into a writable nucleic acid are not efficient polymerase substrates, especially many unnatural nucleobases, preventing the ability to effectively use a polymerase to generate long strands of the nucleic acid polymer. In a chemical synthesis and ligation approach, short writable nucleic acid polymers are constructed on a DNA synthesizer, which can be done utilizing phosphoramidite synthesis protocols, typically resulting in polymer lengths of 10-200 nucleotides. To assist in ligation, in some embodiments, the short-synthesized polymer further comprises a 5′-phosphate group and a native unaltered 3″-hydroxyl group. A DNA ligase enzyme in the presence of ATP (e.g., T4 DNA ligase) will join the short polymers together to generate a long repeating polymer. In some embodiments, a complementary “splint” nucleic acid oligonucleotide that can hybridize to the reactive ends is utilized to assist ligation.

In some embodiments, to generate a double-stranded writable nucleic acid, a nucleic acid complement comprising a 5′-phosphate group is synthesized. Prior to ligation, the complement strand hybridizes with the writable nucleic acid. In some embodiments, hybridization of the complement strand results in a duplex with sticky ends that can be efficiently ligated into a double-stranded writable nucleic acid polymer utilizing a ligase enzyme and ATP.

Ligation-derived polymer molecules may result in a range of polymer lengths. In some embodiments, a mixture of polymers with variable lengths is used for data encoding. In some embodiments, a specific length is enriched and/or isolated (e.g., by electrophoresis) and used for data encoding.

Several embodiments are directed to polymerase expansion of writable nucleic acid polymers via repetitive expansion using a thermostable polymerase (e.g., DNA polymerase from Thermococcus litoralis). In such embodiments, a sensitizer may be conjugated to the thermostable polymerase. For more on polymerase expansion of repetitive regions, see J. S. Hartig and E. T. Kool (J. S. Hartig and E. T. Kool, Nucleic Acids Res. 2005; 33; 4922-7, the disclosure of which is incorporated herein by reference).

If the ends of the data field DNA to be ligated are inefficient as a ligase enzyme substrate because of poor hybridization or an unnatural structure that interferes with the enzyme, in accordance with various embodiments, natural nucleobases can be added at ligation sites to ensure a good hybridization/ligation. In some embodiments, chemical ligation is utilized to generate a writable nucleic acid polymer. Chemical ligation can be achieved with cyanogen bromide, with carbodiimide reagents, or by nucleophilic reaction of a phosphorothioate group on one nucleic acid polymer strand terminus and a leaving group, such as (for example) iodide, on the other nucleic acid polymer strand terminus. Although chemical ligation involves joining of a phosphate end to a hydroxyl end, the reaction may be carried out with a 5′-phosphate and 3′-hydroxyl, or a 3′-phosphate and a 5′-hydroxyl. Such methods of chemical ligation have been described (see E. T. Kool, Acc Chem Res. 1998; 31; 502-510; C. Obianyor, et al., Chembiochem. 2020; 21; 3359-3370; and Y. Xu and E. T. Kool, Nucleic Acids Res. 1999; 27; 875-81; the disclosures of which are each incorporated herein by reference).

Described herein are various embodiments of nucleic acid polymers for encoding data, comprising: a plurality of convertible residues iteratively spaced (e.g., approximately regularly spaced) along and covalently linked to the backbone of the nucleic acid polymer, wherein each of the plurality of convertible residues has a first state and is capable of being converted from the first state into a second state, the first state and the second state being different: wherein the plurality of convertible residues are covalently linked to the nucleic acid polymer in the first and in the second state; and wherein the nucleic acid polymer comprises a sequence at the 3′ end of the nucleic acid polymer for priming a polymerase. In some embodiments, the nucleic acid polymer is a single-stranded nucleic acid polymer. In some embodiments, the sequence is a unique sequence only present at the 3′ end of the nucleic acid polymer. In some embodiments, the nucleic acid polymer comprises Deoxyribonucleic acid (DNA). Ribonucleic acid (RNA), phosphorothioate DNA, glycerol nucleic acids (GNA), threose nucleic acids (TNA), locked nucleic acids (LNA), or a combination thereof. In some embodiments, the nucleic acid polymer comprises greater than 10 convertible residues. In some embodiments, the ratio of the total number of nucleotides of the convertible residues in the nucleic acid polymer is between 2 to 100. In some embodiments, the plurality of convertible residues are non-naturally occurring nucleobases. In some embodiments, the plurality of convertible residues are modified naturally occurring nucleobases or derivatives of naturally occurring nucleobases. In some embodiments, each of the plurality of convertible residues comprises a chemically modifiable moiety. In some embodiments, each of the plurality of convertible residues, the chemically modifiable moiety is directly attached to the base of the convertible residue. In some embodiments, each of the plurality of convertible residues the chemically modifiable moiety is attached to the base without a linker or a sidechain. In some embodiments, the plurality of convertible residues are covalently linked to the backbone of the nucleic acid polymer via the sugar. In some embodiments, the chemically modifiable moiety is activatable by light, voltage, enzymatic agent, chemical reagent, or a redox agent, thereby converting from the first state into the second state. In some embodiments, the chemically modifiable moiety is activatable by light, thereby converting from the first state into the second state. In some embodiments, the conversion from the first state into the second state occurs via an irreversible reaction. In some embodiments, the convertible residues becomes a naturally occurring nucleobase after conversion into the second state. In some embodiments, the nucleic acid polymer comprises two or more different sets of convertible residues, each set of convertible residues has a first state and is capable of being converted from the first state into a second state, the first state and the second state being different. In some embodiments, each of the plurality of convertible residues comprises a chemically modifiable moiety that can be activated by light. In some embodiments, the two or more different sets of convertible residues are activatable by light of different wavelengths. In some embodiments, a first set of convertible residues is activatable by light of a first wavelength, and a second set of convertible residues is activatable by light of a second wavelength, the first wavelength and the second wavelength being different. In some embodiments, the chemically modifiable moiety comprises one or more photo-removable groups. In some embodiments, chemically modifiable moiety is a leaving group. In some embodiments, the convertible residues are:

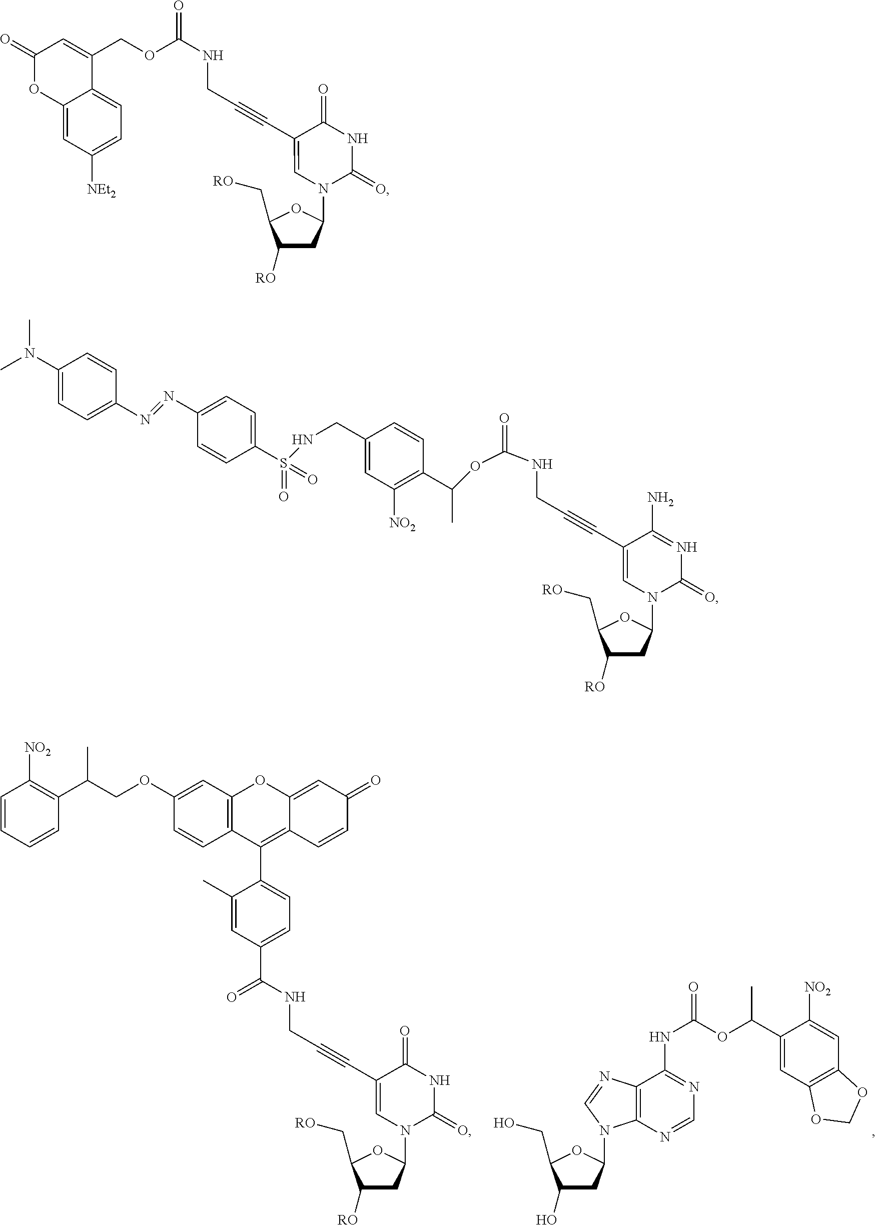

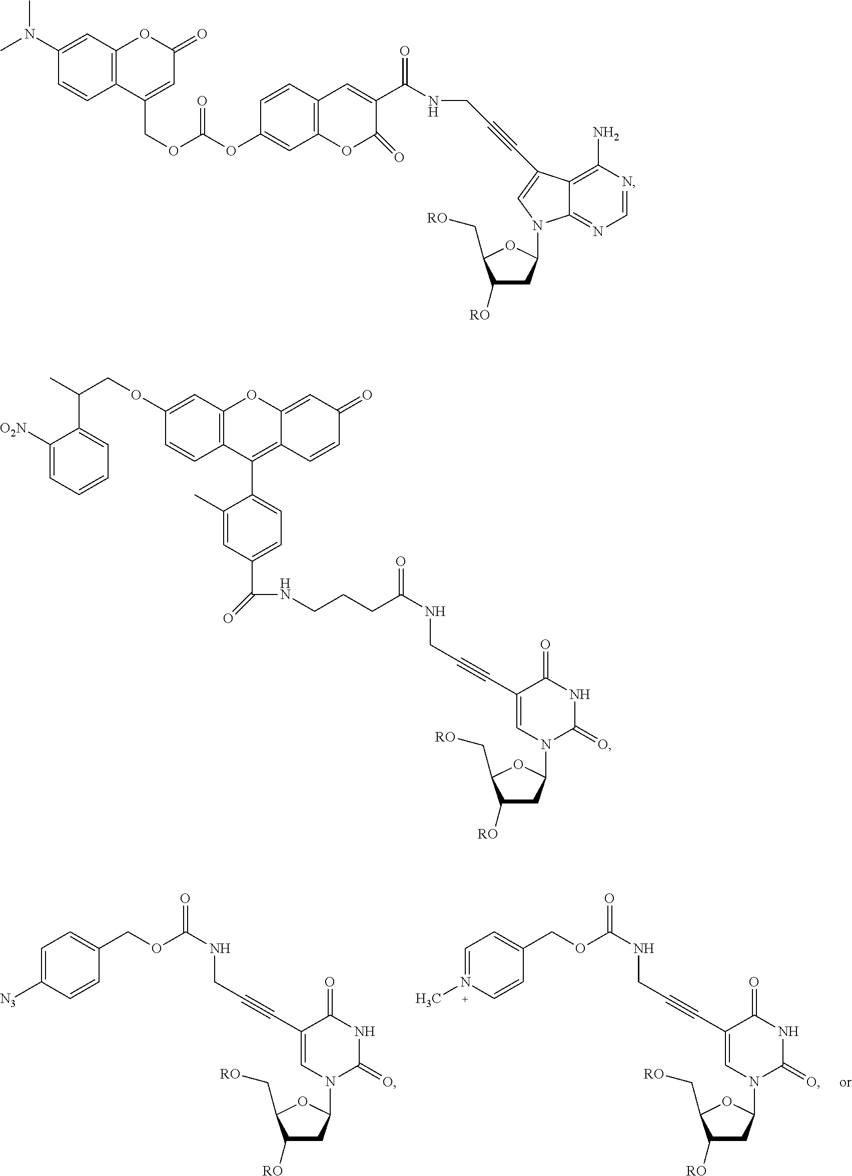

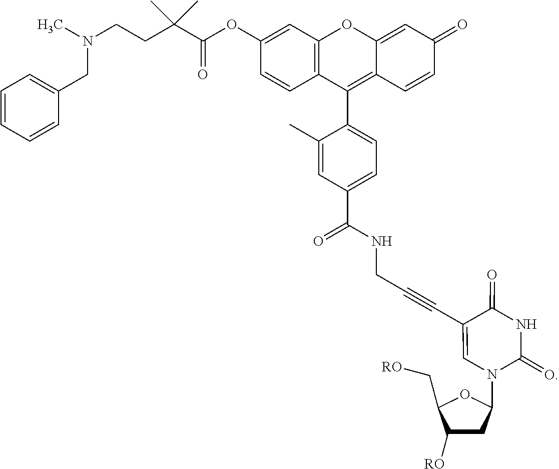

In some embodiments, all the plurality of convertible residues in the nucleic acid polymer have the same structure. In some embodiments, the plurality of convertible residues are capable of being converted by light of a wavelength of 325 nm, 360 nm, or 400 nm. In some embodiments, the plurality of convertible residues are capable of being converted by light of a wavelength of between 400 nm to 850 nm. In some embodiments, each of the plurality of convertible residues comprises a chemically modifiable moiety that is activatable by redox. In some embodiments, the chemically modifiable moiety is capable of being activated by localized oxidation. In some embodiments, the chemically modifiable moiety is capable of being activated by oxidation using electrodes. In some embodiments, the first state and the second state of the plurality of convertible residues are readable by sequencing. In some embodiments, the first state and the second state of the plurality of convertible residues are readable by nanopore sequencing. In some embodiments, the first state and the second state of the plurality of convertible residues are readable by sequencing by synthesis. In some embodiments, each of the plurality of convertible residues is capable of being independently and selectively converted. In some embodiments, the nucleic acid polymer further comprises a plurality of spacer residues linked via the backbone of the nucleic acid polymer, wherein each of the plurality of convertible residues are separated by one or more spacer residues of the plurality of spacer residues. In some embodiments, the iterative spacing among the plurality of convertible residues conforms to a resolution of a writing mechanism for encoding data on the nucleic acid polymer. In some embodiments, the resolution of the writing mechanism is at least 1 nm. In some embodiments, the plurality of spacer residues do not interfere with reading of the convertible residues. In some embodiments, the plurality of spacer residues in the nucleic acid polymer are the same spacer residues. In some embodiments, the plurality of spacer residues comprise two or more different types of spacer residues. In some embodiments, the nucleic acid polymer consists essentially of spacer residues. In some embodiments, each of the plurality of convertible residues are separated by 2, 3, 4, 5, 6, 7, 8, 9, 10, 20, 30, 40, or 50 spacer residues. In some embodiments, the plurality of spacer residues are naturally occurring nucleobases. In some embodiments, the nucleic acid polymer further comprises one or more delimiters linked to the backbone of the nucleic acid polymer. In some embodiments, each of the one or more delimiters comprises one or more naturally occurring nucleobases or non-naturally nucleobases. In some embodiments, the one or more delimiters comprise naturally occurring nucleobases. In some embodiments, the one or more delimiters separate two or more adjacent data fields within the nucleic acid polymer. In some embodiments, the nucleic acid further comprises one or more data tags. In some embodiments, the one or more data tags comprise one or more naturally occurring nucleobases or non-naturally nucleobases. In some embodiments, the one or more data tags are present at the 5′ or 3′ end of the nucleic acid polymer. In some embodiments, the one or more data tags are incorporated to the nucleic acid polymer during synthesis of the nucleic acid polymer, during conversion of the plurality of convertible residues to the second state, or via ligation after the plurality of convertible residues are converted to the second state.

Described herein are various embodiments of a polymerase for use in encoding data into a nucleic acid polymer, comprising: a nucleic acid polymerase conjugated with a sensitizer, wherein the sensitizer is molecule that can capture and transmit light or redox energy. In some embodiments the sensitizer is conjugated to the polymerase via cysteine side-chains. In some embodiments the sensitizer has a structure of:

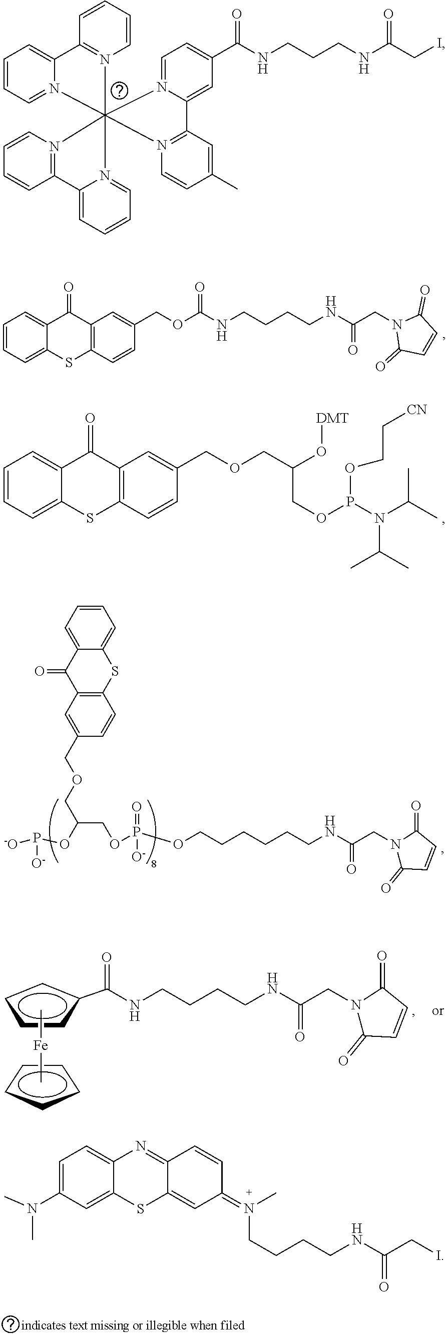

In another aspect, provided herein are systems and methods for writing or reading the writable or written polymers provided herein (e.g., nucleic acid polymers).

In another aspect, provided herein are systems for data writing, comprising: a writable polymer comprising a plurality of convertible residues iteratively spaced along and covalently linked to the backbone of the polymer, wherein each of the plurality of convertible residues has a first state and is capable of being converted from the first state into a second state, the first state and the second state being different and the plurality of convertible residues in the first state and the second state are readable by polymerase: wherein the plurality of convertible residues are attached covalently linked to the polymer in the first state and in the second state; and a data writing device for writing data on the writable polymer.

In some embodiments, the writable polymer is a writable nucleic acid polymer, and the plurality of convertible residues are convertible nucleobases. In some embodiments, the data writing device comprises a nanopore. In some embodiments, the data writing device converts the plurality of convertible residues into the second state by light pulses, voltage pulses, an enzymatic agent, or a redox agent. In some embodiments, the data writing device converts the converts the plurality of convertible residues into the second state by light pulses. In some embodiments, the data writing device comprises a light irradiation device.

Described herein are various embodiments of a system for data writing, the system comprising: a writable nucleic acid polymer comprising a plurality of convertible residues iteratively spaced (e.g., approximately regularly spaced) along and linked to the backbone of the nucleic acid polymer, wherein each of the plurality of convertible residues has a first state and is capable of being converted from the first state into a second state, the first state and the second state being different, wherein the plurality of convertible residues are covalently linked to the nucleic acid polymer in the first state and in the second state, and wherein the nucleic acid polymer comprises a sequence for priming a polymerase at the 3′ end of the nucleic acid polymer; and an enzyme conjugated with a sensitizer, wherein the sensitizer receives and transmits energy to the convertible residues. In some embodiments, the system further comprises an energy source for providing light or redox energy. In some embodiments, the energy source provides light. In some embodiments, the energy source provides redox energy. In some embodiments, the transmission of energy from the sensitizer to the convertible residues converts the convertible residues from the first state to the second state. In some embodiments, the convertible residues are:

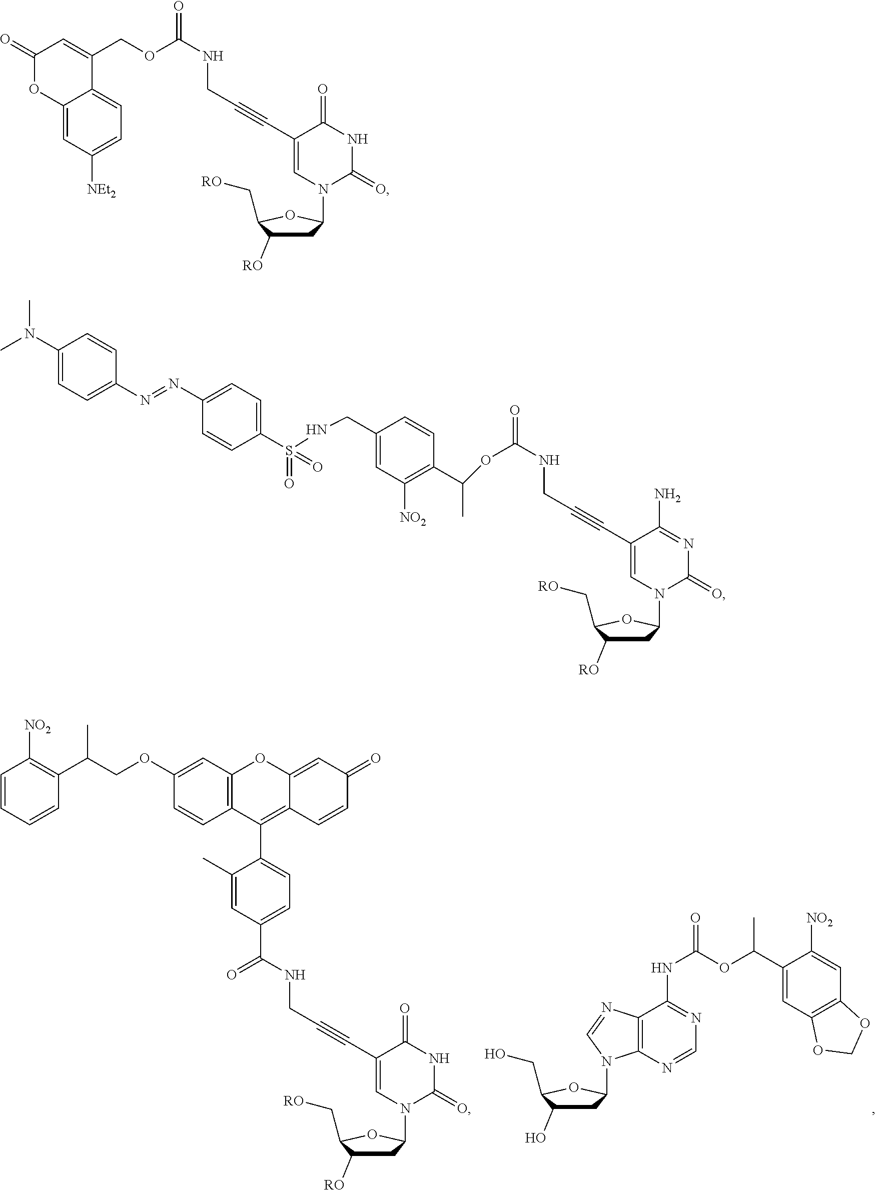

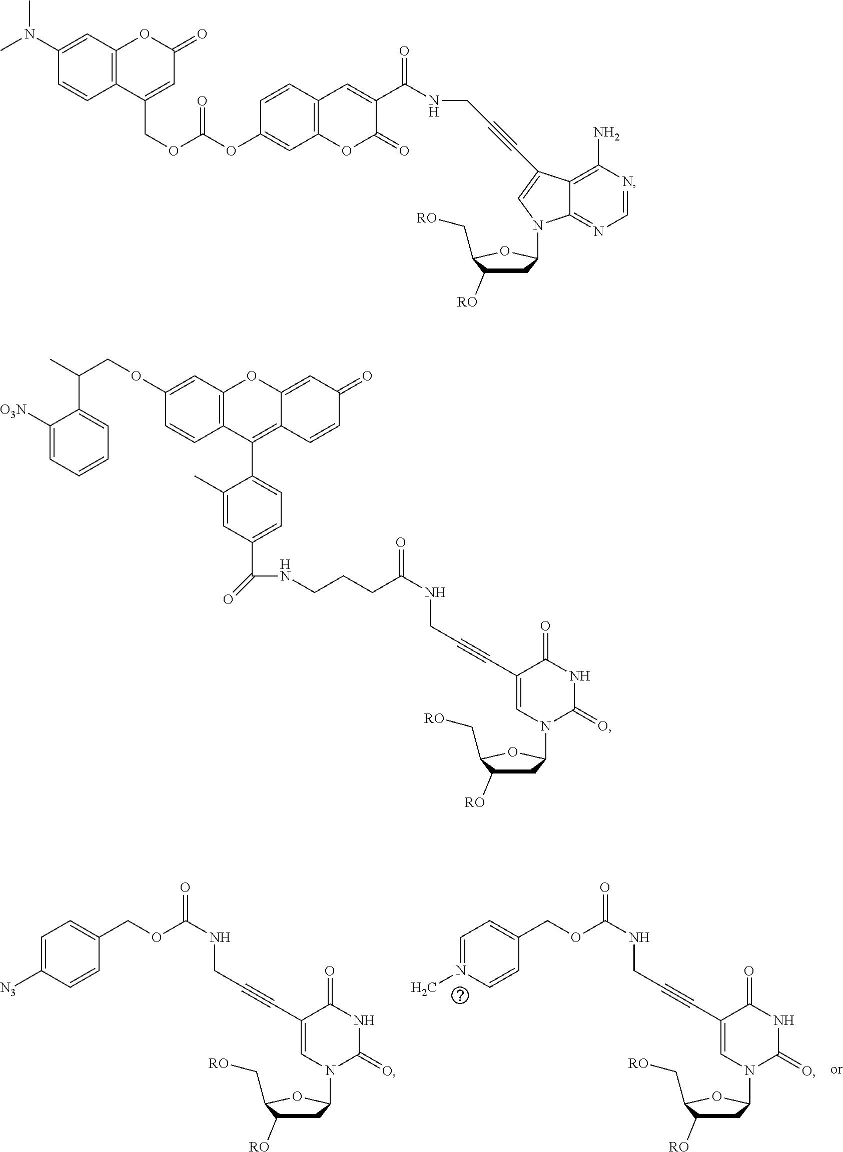

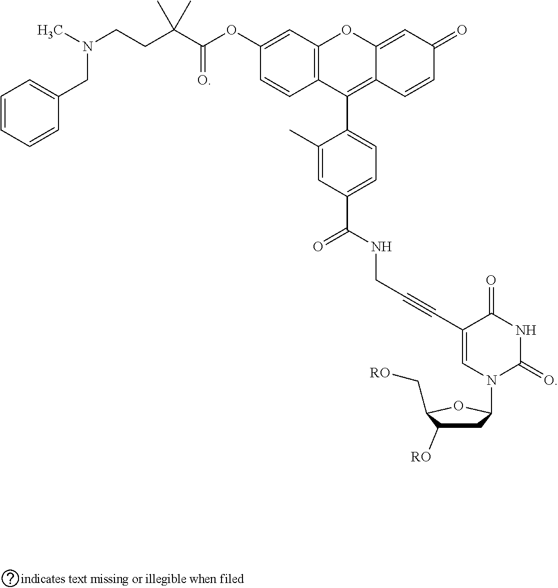

In some embodiments, the enzyme binds the writable nucleic acid polymer. In some embodiments, the enzyme is a polymerase. In some embodiments, the enzyme is a nucleic acid polymerase. In some embodiments, the enzyme is a template-dependent DNA polymerase. In some embodiments, the enzyme is a template-independent DNA polymerase. In some embodiments, the enzyme is a nuclease. In some embodiments, the enzyme is a helicase. In some embodiments, the enzyme is a nickase. In some embodiments, the sensitizer has a structure of:

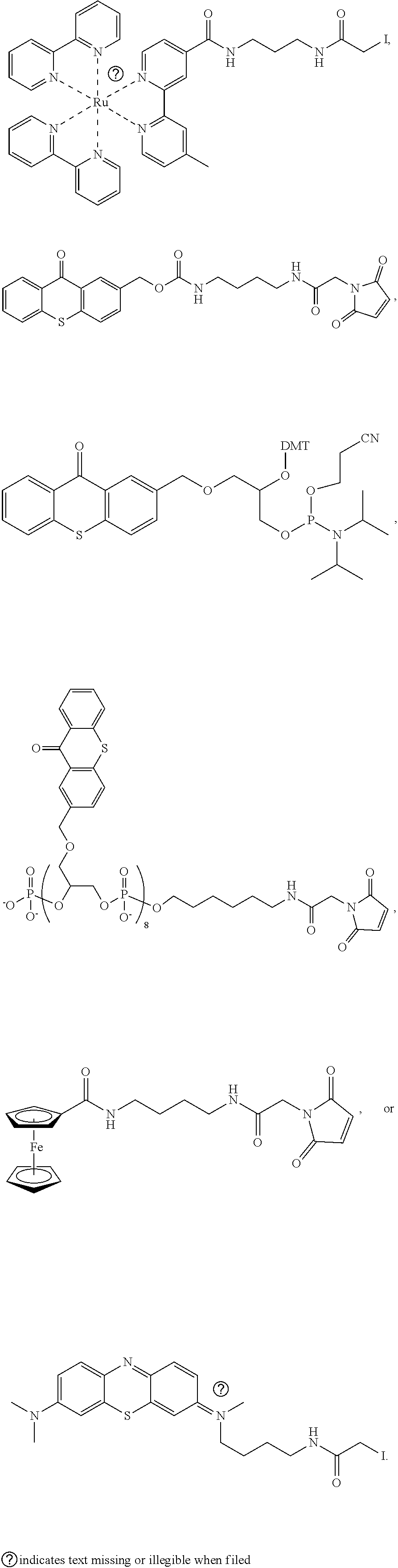

In some embodiments, the sensitizer is conjugated to the enzyme via a cysteine sidechain. In some embodiments, the system further comprises a primer oligomer, wherein the primer oligomer has a sequence complementary to the sequence at the 3′ end of the nucleic acid polymer. In some embodiments, the enzyme produces a nucleic polymer complementary to the writable nucleic acid polymer when writing data on to the writable nucleic acid polymer. In some embodiments, the system, further comprises a set of triphosphate residues. In some embodiments, the triphosphate residues are dNTPs or NTPs. In some embodiments, the triphosphate residues are modifiable dNTPs or NTPs.

In yet another aspect, provided herein are methods for writing data onto a writable polymer, comprising: providing a writable polymer that comprises a plurality of convertible residues iteratively spaced along and covalently linked via the backbone of the polymer, wherein each convertible residues of the plurality of convertible residues has a first state and is capable of being converted from the first state into a second state, the first state and the second state being different and the plurality of convertible residues in the first state and the second state are readable by polymerase; and selectively converting, utilizing a data writing device, one or more of the plurality of convertible residues into the second state such that a data encoded polymer is generated. In such embodiments, a sensitizer may be conjugated to the polymerase.

Various embodiments as described herein are directed towards writing and reading data on nucleic acid polymers. In some embodiments, a writable nucleic acid polymer is provided having convertible residues iteratively spaced along the polymer. The provided writable nucleic acid polymer may also have spacers, delimiters, and data tags, as described herein. To write data upon a nucleic acid polymer, in accordance with various embodiments, an individual strand is passed through a device having a nanopore. The device having a nanopore further provides a method for selectively converting a convertible residue from a first state into a second state. A number of systems, devices and/or methods can be utilized for converting a convertible residue, including (but not limited to) light pulses, voltage pules, an enzymatic agent, a chemical reagent, and/or a redox agent. An example of a nanopore device for passing DNA through and encoded with localized light pulses is described within the examples provided in the Examples.

In some embodiments, the writable polymer is a writable nucleic acid polymer, and the plurality of convertible residues are convertible nucleobases. In some embodiments, the data writing device comprises a nanopore, and the method further comprising passing the writable polymer through the nanopore of the writing device, wherein the nanopore comprises converts one or more of the plurality of convertible residues into the second state.

In some embodiments, the nanopore is a plasmonic nanopore that provides light pulses or redox energy to selectively convert convertible residues from the first state into the second state. In some embodiments, the data writing device comprises a plasmonic well or channel, and the method further comprising transferring the writable polymer into the plasmonic well or channel of the data encoding device, wherein the plasmonic well or channel provides light pulses or redox energy to selectively convert convertible residues from the first state into the second state. In some embodiments, the data writing device selectively coverts the convertible residues into the second state by light pulses, voltage pulses, an enzymatic agent, or a redox agent. In some embodiments, the data writing device selectively converts the converts the convertible residues into the second state by light pulses.

In some embodiments, the convertible residues become naturally occurring nucleobases after conversion into the second state.

In some embodiments, the plurality of convertible residues (e.g., residues comprising chemically alterable groups) comprise two or more types of convertible residues, wherein a first type of convertible residues are activatable by light of a first wavelength and a second type of convertible residues are activatable by light of a second wavelength. In some embodiments, the iterative spacing among the plurality of the convertible residues conforms to a resolution of the data writing device for selectively converting the convertible residues. In some embodiments, the selectively converting step does not require specific positioning of the writable polymer. In some embodiments, the conversion of the convertible residues into the second state is non-uniform on the data encoded polymer. In some embodiments, the conversion of the convertible residues into the second state is not limited to certain positions on the data encoded polymer.

In some embodiments, the method further comprising stretching or combing the writable polymer (e.g., a writable DNA) on a solid support.

In some embodiments, the method further comprising visualizing locations of the convertible residues using a dye.

In some embodiments, the method further comprising locally illuminating the writable polymer. In some embodiments, the locally illuminating uses Stimulated Emission Depletion (STED) laser.

In some embodiments, the method further comprising joining two or more data fields from two or more writable polymers end-to-end, resulting in a joined polymer comprising two or more data fields.

In some embodiments, the method further comprising controlling the passage rate of the writable polymer through the nanopore of the writing device.

In some embodiments, a plurality of writable polymers pass through the data writing device to write the same data (e.g., generating data redundancy).

In some embodiments, to encode data on a writable nucleic acid polymer provided herein, in accordance with various embodiments, an individual polymer has light energy or redox energy impinged upon the polymer in an iterative fashion such that it can controllably and selectively convert the convertible residues to encode a data code (e.g., a binary data code).

Although a device with a nanopore is described, any device that can controllably and selectively convert the convertible residues in accordance with a data code. In some embodiments, the device utilizes plasmonic channels or plasmonic wells for controllably and selectively converting the convertible residues.

Described herein are various methods for writing data onto a writable nucleic acid polymer, comprising: (a) providing in a solution a writable nucleic acid polymer that comprises a plurality of convertible residues iteratively spaced along and linked to the backbone of the nucleic acid polymer, wherein each of the plurality of convertible residues has a first state and is capable of being converted from the first state into a second state, the first state and the second state being different, wherein the plurality of convertible residues are covalently linked to the nucleic acid polymer in the first state and in the second state, and wherein the nucleic acid polymer comprises a sequence for priming a polymerase at the 3′ end of the nucleic acid polymer; and (b) providing an enzyme conjugated with a sensitizer that receives and transmits energy to the convertible residues; and (c) selectively converting one or more of the plurality of convertible residues into the second state such that a data encoded polymer is generated. In some embodiments, the transmission of energy from the sensitizer to the convertible residues converts the convertible residues from the first state to the second state. In some embodiments, the transmission of energy from the sensitizer to the convertible residues occurs as the enzyme moves along the writable nucleic acid polymer. In some embodiments, the transmission of energy from the sensitizer to the convertible residues occurs when the enzyme is within proximity to the convertible residues of the writable nucleic acid polymer. In some embodiments, one or more of the plurality of convertible residues are selectively converted into the second state by light pulses, voltage pulses, an enzymatic agent, or a redox potential. In some embodiments, one or more of the plurality of convertible residues are selectively converted into the second state by light. In some embodiments, one or more of the plurality of convertible residues are selectively converted into the second state by a redox potential. In some embodiments, the enzyme binds the writable nucleic acid polymer. In some embodiments, the enzyme is a polymerase. In some embodiments, the enzyme is a nucleic acid polymerase.

Described herein are various methods for writing data onto a nucleic acid polymer, comprising: (a) providing in a solution a template nucleic acid polymer comprising a sequence for priming a polymerase at the 3′ end of the nucleic acid polymer; and (b) providing a polymerase conjugated with a sensitizer that is capable of receiving and transmitting energy; (c) providing a plurality of convertible deoxynucleoside triphosphates (dNTPs), each of the convertible dNTPs comprising a chemically modifiable moiety in a first state and is capable of being converted from the first state into a second state, the first state and the second state being different; and (d) incorporating the convertible dNTPs in the solution by the polymerase (e.g., synthesis reaction) while selectively converting one or more of the plurality of the incorporated convertible dNTPs into a second state by providing energy to the sensitizer, thereby generating a data encoded nucleic acid polymer complementary to the template nucleic acid polymer. In some embodiments, the convertible dNTPs become convertible residues of the generated data encoded nucleic acid polymer complementary to the template nucleic acid polymer. In some embodiments, the sensitizer receives the provided energy and transmits to the convertible dNTPs that have been incorporated (i.e., convertible residues), and converts the convertible residues from the first state to the second state. In some embodiments, the convertible residues are selectively converted when the polymerase is within proximity to the convertible residues. In some embodiments, the convertible residues are selectively converted simultaneously when the convertible dNTPs are incorporated to become convertible residues in the nucleic acid polymer. In some embodiments, one or more of the plurality of convertible residues are selectively converted into the second state by light pulses, voltage pulses, an enzymatic agent, or a redox potential. In some embodiments, one or more of the plurality of convertible residues are selectively converted into the second state by light. In some embodiments, one or more of the plurality of convertible residues are selectively converted into the second state by a redox potential.

Described herein are various methods for writing data onto a nucleic acid polymer, comprising: (a) providing an polymerase conjugated with a sensitizer capable of receiving and transmitting energy: (b) providing a primer and a plurality of convertible deoxynucleoside triphosphates (dNTPs), each of the convertible dNTPs comprising a chemically modifiable moiety in a first state and is capable of being converted from the first state into a second state, the first state and the second state being different; and (c) incorporating the convertible dNTPs in the solution by the polymerase (e.g., synthesis reaction) while selectively converting one or more of the plurality of the incorporated convertible dNTPs into a second state by providing energy to the sensitizer, thereby generating a data encoded nucleic acid polymer. In some embodiments, the convertible dNTPs become convertible residues of the generated data encoded nucleic acid polymer. In some embodiments, the polymerase is a template-independent polymerase. In one embodiment, the polymerase is terminal deoxynucleotidyl transferase (TdT). In some embodiments, the sensitizer receives the provided energy and transmits to the convertible dNTPs that have been incorporated (i.e., convertible residues), and converts the convertible residues from the first state to the second state. In some embodiments, the convertible residues are selectively converted when the polymerase is within proximity to the convertible residues. In some embodiments, the convertible residues are selectively converted simultaneously when the convertible dNTPs are incorporated to become convertible residues in the nucleic acid polymer. In some embodiments, one or more of the plurality of convertible residues are selectively converted into the second state by light pulses, voltage pulses, an enzymatic agent, or a redox potential. In some embodiments, one or more of the plurality of convertible residues are selectively converted into the second state by light. In some embodiments, one or more of the plurality of convertible residues are selectively converted into the second state by a redox potential. In some embodiments, the enzyme binds the writable nucleic acid polymer.

In some embodiments, the convertible residues are selected from:

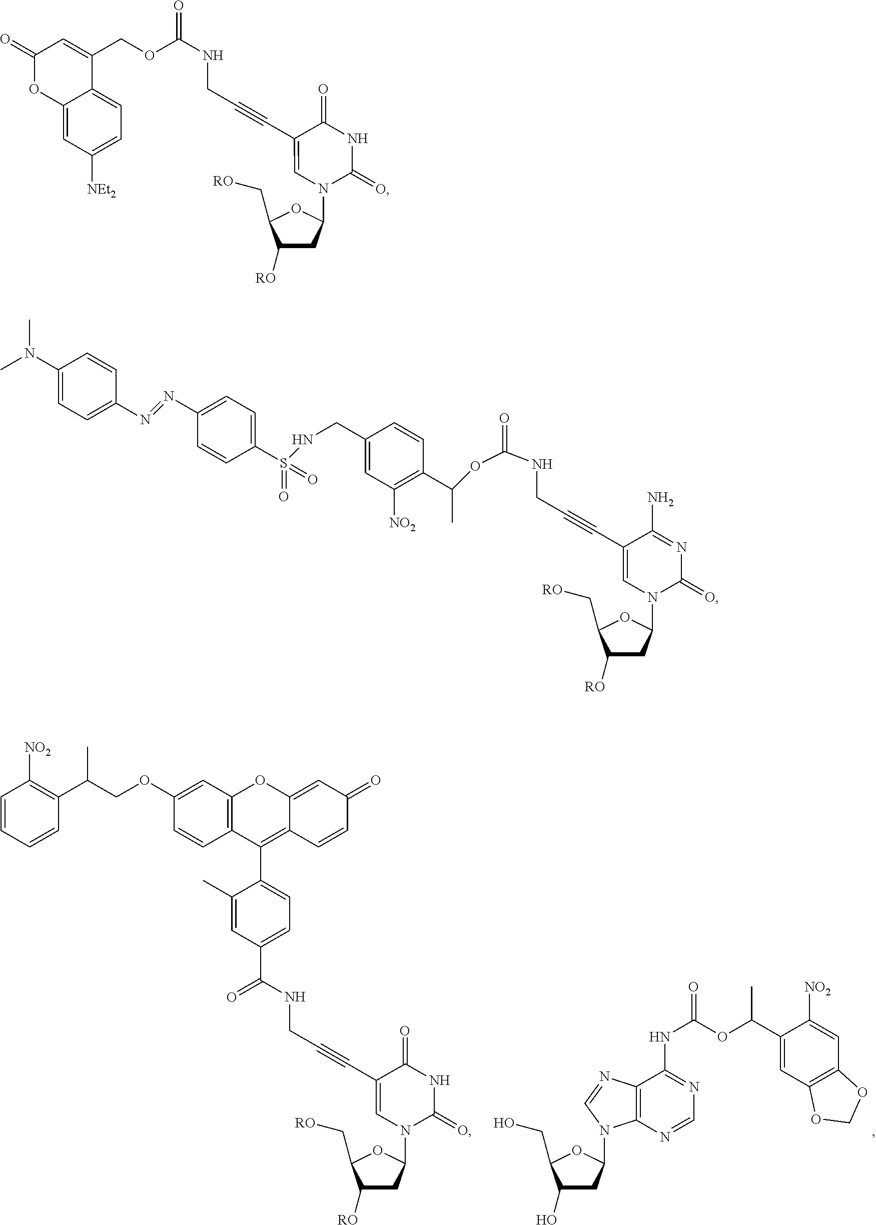

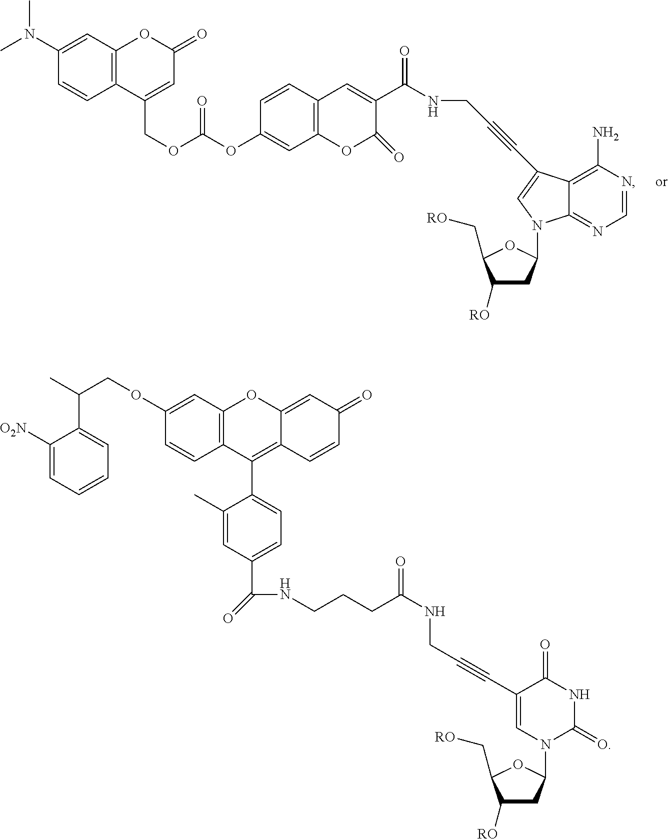

In some embodiments, the sensitizer has a structure selected from:

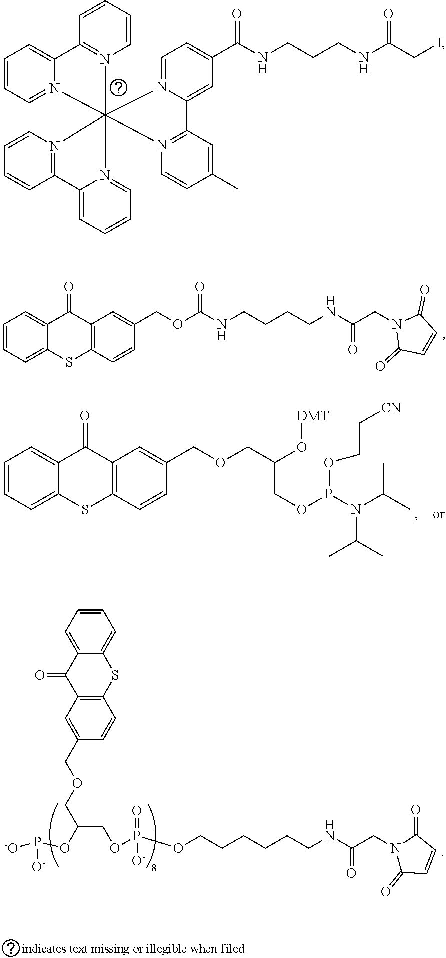

In some embodiments, the sensitizer is conjugated to the enzyme via a cysteine sidechain. In some embodiments, the method further comprises: (c′) adding to the solution a primer oligomer, wherein the primer oligomer has a sequence complementary to the sequence at the 3′ end of the nucleic acid polymer. In some embodiments, the method further comprises: (c″) adding to the solution a set of triphosphate residues, wherein the set of triphosphate residues comprises triphosphate residues having a first structure and triphosphate residues having a second structure. In some embodiments, the set of triphosphate residues are added such that a final concentration of triphosphate residues having the first structure is lower than a final concentration of triphosphate residues having the second structure. In some embodiments, the ratio of the triphosphate residues having the first structure to the triphosphate residues having the second structure results in the enzyme pausing as the enzyme moves along writable nucleic acid polymer and reaches residues of the template complementary to the triphosphate residues having the first structure.

### DATA WRITING INTO NUCLEIC ACIDS WITH SENSITIZERS CONJUGATED TO POLYMERASES

Several embodiments are directed towards writing data on nucleic acid polymers utilizing sensitizers conjugated to polymerases. In many embodiments, a writable nucleic acid polymer is provided comprising convertible residues iteratively spaced along the polymer. The provided writable nucleic acid polymer may also have spacers, delimiters, and data tags, as described herein. To write data upon a nucleic acid polymer, in accordance with various embodiments, the polymer is provided in a buffered solution with polymerase comprising a conjugated sensitizer, dNTPs, and primer. In many embodiments, the polymerase with conjugated sensitizer is utilized in conjunction with a light or redox source for selectively performing alteration of convertible residues from a first state into a second state. Examples of data encoding with a polymerase with conjugated sensitizer and light pulses or redox is described within the examples provided in the Examples.

In some embodiments, as the polymerase with conjugated sensitizer travels along a writable nucleic acid polymer, the sensitizer molecule in conjunction with light or redox selectively alters the convertible residues. For instance, if a convertible residue is to be altered into a second state via light pulses, as the polymerase with a conjugated light sensitizer reaches the group, requisite light is provided (e.g., requisite wavelength and intensity), resulting in only the convertible residues within resolution of the sensitizer to be altered. If a convertible residue is to remain in a first state, as the polymerase with a conjugated light sensitizer reaches the group, light will not be provided at the requisite conditions and the group will not be converted. In many embodiments, to ensure the sensitizer converts only the local convertible residues, the convertible residues can be flanked with spacers in accordance with the sensitizer's resolution.

In certain embodiments, as a writable nucleic acid polymer passes through the nanopore, the device selectively provides the mechanism for converting the convertible residue. For instance, if a nucleobase is to be converted into a second state via light pulses, as the nucleic acid polymer passes through the nanopore, the device can provide light such that it only contacts the convertible residue to be converted. If a nucleobase is to remain in a first state, the device will not provide light such that the convertible residue will pass through the nanopore without conversion. In many embodiments, to ensure a device only converts a single nucleobase, the convertible residue can be flanked with spacers in accordance with the device's writing resolution. For instance, if an optical light source and device with 1 nm of resolution is used to alter nucleobases, then each convertible base needs to be separated by at least 1 nm.

In certain embodiments, if a nucleobase is to be converted into a second state via light pulses, as the nucleic acid polymer passes through the nanopore, the device can provide light such that it only contacts the set of convertible residues to be converted. If a nucleobase is to remain in the initial state, the device will not provide light such that the convertible residue will pass through the nanopore without conversion. In many embodiments, to ensure a device only converts a set of nucleobase, the set of convertible residues can be flanked with spacers in accordance with the device's writing resolution.

In some embodiments, to ensure a device only converts a single nucleobase (or a set of nucleobases), the device utilizes two or more mechanisms for converting a nucleobase; a first system, method, or device being able to convert a first nucleobase structure but not a second nucleobase structure and a second system, method, or device being able to convert the second nucleobase structure but not the first nucleobase structure. For instance, a device can utilize two wavelengths of light for providing energy such that the first wavelength is able to convert a first nucleobase structure but not a second nucleobase structure and a second wavelength is able to convert the second nucleobase structure but not the first nucleobase structure.

In some embodiments, to ensure a device only converts a single nucleobase (or a set of nucleobases), the device utilizes two or more system, method, or device for converting a nucleobase; a first mechanism being able to convert a first nucleobase structure but not a second nucleobase structure and a second mechanism being able to convert both the first nucleobase structure and the second nucleobase structure concurrently as a pair. For instance, a device can utilize two wavelengths of light for providing energy such that the first wavelength is able to convert a first nucleobase structure but not a second nucleobase structure and a second wavelength is able to convert both the first nucleobase structure and the second nucleobase structure concurrently as a pair.

In many embodiments, the writing device is provided a code for writing the data into the nucleic acid polymer. Accordingly, the writing device will selectively convert various nucleobases of the polymer that are akin to being a “1” in binary code, while selectively allowing nucleobases of the polymer to pass through the pore without conversion that are akin to being a “0”. After writing a data code into the nucleic acid polymer, it can be stored by any appropriate method, system, or device for storing nucleic acid molecules. For instance, data written nucleic acid polymers can be stored dry, as a precipitate, or in an appropriate nuclease-free solution at room temperature, or at colder temperatures (e.g., −20° C.). Stabilizers such as (for example) alcohol, chelating agents and nuclease inhibitors, may be included with the stored nucleic acid.

In some embodiments, the polymers provided herein (e.g., nucleic acid polymers) can be stored under standard nucleic acid storage protocols. In some embodiments, the polymer is a nucleic acid polymer that can be stored in appropriate nuclease-free solution at room temperature, or at a lower temperature (e.g., −20° C.). In some embodiments, the polymer can be stored at room temperature without stabilizer.

In many embodiments, the data encoding device is provided a code for writing the data into the nucleic acid polymer. Accordingly, in some embodiments, the encoding device will selectively convert various nucleobases of the polymer that in accordance with the code. In some embodiments that use solitary nucleobases as a bit, a data is encoded by selecting converting some of the nucleobase and selectively not converting the others, resulting in a binary code of converted and unconverted nucleobases. In some embodiments that use solitary nucleobases as a bit, a data is encoded by selectively converting some of the nucleobase into a first converted structure and selectively converting others into a second converted structure, resulting in a binary code of converted nucleobases; any unconverted nucleobases remain unencoded and are not utilized to decode the data code.

In some embodiments that utilize a set of nucleobases to encode a bit, each set will comprise at least two convertible residues and the encoding device will selectively convert a first nucleobase of some of the sets into a converted structure and selectively convert a second nucleobase of other sets into a converted structure, resulting in a binary code. In some embodiments that utilize a set of nucleobases to encode a bit, each set will comprise at least two convertible residues and the encoding device will selectively convert a first nucleobase of some of the sets into a converted structure and selectively convert both nucleobases of other sets into a converted structure, resulting in a binary code.

Any appropriate nucleic acid polymerase capable of conjugating with a sensitizer and traveling along the nucleic acid polymer can be utilized. In theory, any nucleic acid polymerase can be utilized. Examples of polymerases that can be used include (but are not limited to) Taq DNA polymerase, KlenTaq DNA polymerase, Klenow fragment of DNA Pol I (Kf), T7 DNA polymerase. T4 DNA polymerase, KOD DNA polymerase, 90N DNA polymerase, Phi29 DNA polymerase. Bst DNA polymerase, HIV reverse transcriptase, Vent DNA polymerase, and SuperScript polymerase. In many embodiments, the sensitizer group is conjugated onto any available amino acid within 10 Å of the DNA when in contact with the DNA. An available acid is any amino acid that is capable of being conjugate such that the polymerase can still polymerize a nucleic acid template efficiently. For more discussion on polymerases and sensitizer conjugation, see, e.g., A. F. Garnder, et al., Front Mol Biosci. 2019; 6:28; and A. Hottin and A. Marx, Acc Chem Res. 2016:49:418-27: the disclosures of which are each herein incorporated by reference.

In many embodiments, the light source works in conjunction with a provided code for writing the data into the nucleic acid polymer. Accordingly, the light source will provide the requisite light to selectively convert various convertible residues of the polymer. After writing a data code into the nucleic acid polymer, it can be stored by any appropriate method, system, or device for storing nucleic acid molecules. For instance, data written nucleic acid polymers can be stored dry, as a precipitate, or in an appropriate nuclease-free solution at room temperature, or at colder temperatures (e.g., −20° C.). Stabilizers such as (for example) alcohol, chelating agents and nuclease inhibitors, may be included with the stored nucleic acid.

In some embodiments, nucleic acid polymers most efficiently store data at the single molecule level, providing the highest potential density of information. In some embodiments, however, if redundancy of data is required for better accuracy of data storage, then a plurality of nucleic acid polymers could be used to redundantly write the same data on each polymer of the plurality. Error correction algorithms are already well developed for digital data storage, and some of these algorithms can be applied in the present approach (see J. Li, et al., IEEE Transactions on Emerging Topics in Computing. 2021; 9; 651-663, the disclosure of which is incorporated herein by reference).

In various embodiments in which the encoded data is to be decoded by sequencing by synthesis (SBS), it may be desirable to have a redundancy of data and thus the same data on each polymer of the plurality. For instance, when using a nucleobase structure such as O6-nitrobenzyl-guanine, the structure is read as a mix of A and G using SBS and thus a redundancy of reading the structure would be needed to interpret whether the structure is O6-nitrobenzyl-guanine, guanine, or adenine.

In another aspect, also provided herein are methods for reading data from a polymer encoded with data, comprising: providing the polymer encoded with data comprising convertible residues iteratively spaced along and covalently linked via the backbone of the polymer, wherein a first subset of the convertible residues are in a first state and a second subset of the convertible residues are in a second state, the first state and the second state being different and the plurality of convertible residues in the first state and the second state are readable by polymerase; and passing the writable polymer encoded with data through a data reading device to read the encoded data on the polymer encoded with data.

In some embodiments, the writable polymer is a writable nucleic acid polymer, and the plurality of convertible residues are convertible residues. In some embodiments, the convertible residues in the first state can be converted into the second state via light. In some embodiments, the data reading device comprises a nanopore. In some embodiments, the data reading device is a sequencing device. In some embodiments, the sequencing device is a sequencing by synthesis device.

In some embodiments, the method further comprising measuring current flow of electrolytes during passage of the writable polymer.

In some embodiments, the method further comprising determining whether each of the plurality of convertible residues is in the first state, or the second state based on the measured current flow of electrolytes during passage of the writable polymer.

In some embodiments, the method further comprising re-passing the polymer encoded with data through the data reading device to re-read the encoded data on the polymer encoded with data.

In some embodiments, the method further comprising validating and correcting the encoded data on the polymer encoded with data by comparing the encoded data on multiple copies of the polymer encoded with data.

In another aspect, also provided herein are methods for reading or decoding data from a nucleic acid polymer encoded with data, the method comprising:

providing a plurality of redundant copies of the nucleic acid polymer encoded with data comprising:
a plurality of converted nucleobases, wherein each converted nucleobase comprises a first nucleobase structure, wherein the first converted nucleobase has been converted from a first state into a second state, the first state and the second state being different; and
a plurality of convertible residues, wherein each convertible residue comprising a second nucleobase structure and a directly linked leaving group, and wherein the convertible residue is provided in a first state and is capable of being converted from the first state into a second state by releasing the second leaving group from the second nucleobase structure, the first state and the second state being different;
wherein the converted nucleobases and convertible residues are linked via the nucleic acid polymer backbone; and
sequencing each redundant copy of the plurality redundant copies of the nucleic acid polymer.

In some embodiments, the method further comprising detecting the plurality of converted nucleobases and the plurality of convertible residues; and decoding the data based on the detected plurality of converted nucleobases.

In some embodiments, the plurality of converted nucleobases in the first state and the second state are readable by polymerase. In some embodiments, the plurality of convertible residues in the first state and the second state are readable by polymerase. In some embodiments, the plurality of converted nucleobases and the plurality of convertible residues are detected based on the sequencing result of the redundant copies of the nucleic acid polymer encoded with data.

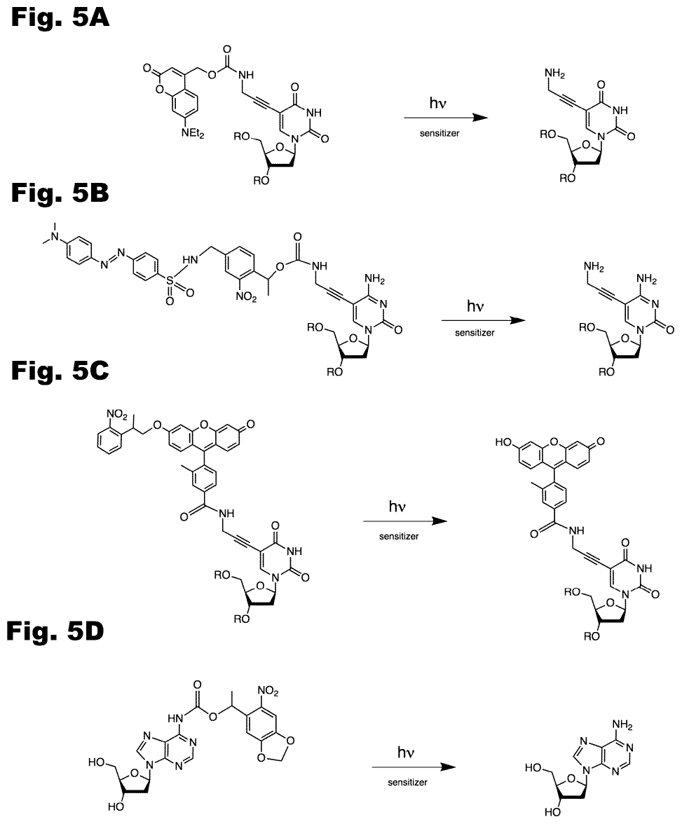

FIG. 4 provides a schematic diagram of writing data via a DNA polymerase and sensitizer group in accordance with various embodiments. In many embodiments, a DNA polymerase enzyme is conjugated with an “antenna” or sensitizer group (marked “S” in FIG. 4), which is highly sensitive to capture of light or of redox signals. In several embodiments, a modifiable DNA template is provided. The modifiable DNA template contains modifiable chemical groups (e.g., chemically modifiable moieties of convertible residues or a redox alterable molecule) along the strand that can be switched structurally by optical excitation and/or by redox (these modifiable chemical groups are represented by star shapes, with alterable photocaged groups therein abbreviated “PC” in FIG. 4). In several embodiments, light pulses or redox are utilized to selectively modify the modifiable chemical groups to yield a sequential pattern of modified groups (e.g., binary code). To yield a sequential pattern, in accordance with many embodiments, the DNA polymerase is provided with a primer that enables the enzyme to start at one end, as well as with dNTPs sufficient to proceed along the strand in a buffer supportive of DNA synthesis. Once synthesis is initiated, the solution containing the enzyme is exposed to pulses of light via LED or laser, or to pulses of redox potential via an electrode. The polymerase's conjugated sensitizer captures this energy, and this energy is transferred to the groups in the DNA template that are closest to the enzyme at the time of the pulse. Differential pulses of energy as the polymerase moves along the template result in a sequence of chemical changes upon the modifiable groups. As the polymerase proceeds further along the template, some chemical groups remained unaltered because the requisite light is not provided. In some embodiments, the polymerase can be induced to pause at specific nucleotides in the sequence by controlling concentrations of dNTPs. For example, lowering the concentration of dGTP relative to the others will result in pauses of the enzyme at cytosine residues in the template. Accordingly, in some embodiments, one or more dNTPs is kept a lower concentration than the other dNTPs in order to induce pausing at particular positions sequence such that the polymerase and sensitizer are localized at that position to induce chemical changes upon the local modifiable chemical group.

Several embodiments are directed to convertible residues (e.g., chemically alterable group or a residue comprising a chemically modifiable moiety), which can be attached a residue and incorporated into a writable nucleic acid polymer. A chemically alterable group, in accordance with various embodiments, is a group that is capable of being converted from a first chemical state into a second chemical state by a controlled reaction chemistry in conjunction with a sensitizer. Any appropriate mechanism to convert a nucleobase from a first state into a second state can be utilized, including (but not limited to) light pulses, voltage, enzymatic agent, chemical reagent, and/or redox. It is understood that residues are not limited to naturally occurring nucleobases, but may also embody unnatural nucleobases, such as designer nucleobases.

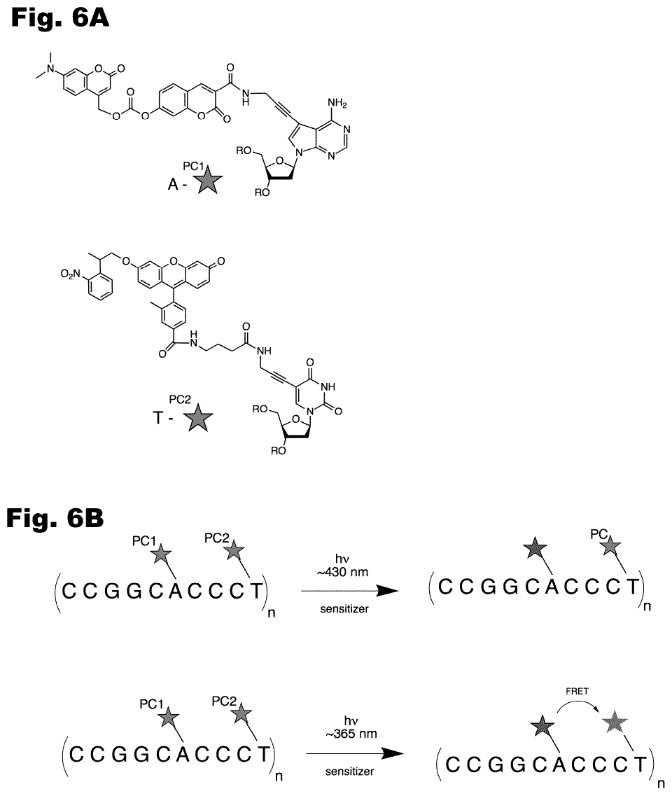

FIGS. 5A to 5D provide examples of convertible residue (e.g., chemically alterable group) that can be incorporated into the template DNA, in accordance with various embodiments. The convertible residues contain chemical bonds that can be cleaved by photoexcitation or by redox. Examples shown here include caged DNA bases, caged fluorophores, and releasable fluorescence quenchers. FIG. 5A provides an example of a convertible residue containing a photocleavable caging group. Removal of the caging group yields an amine-substituted DNA base. FIG. 5B provides an example of a convertible residue containing a photocleavable quencher. Removal of the quencher group yields an amine-substituted DNA base. FIG. 5C provides an example of a convertible residue containing a photocleavable cage and fluorophore. Removal of the caging group yields a fluorescent label on a DNA base. FIG. 5D provides an example of a convertible residue containing a photocleavable cage. Removal of the caging group yields a native DNA base.

Various photo removable groups can be incorporated into convertible residues (see, e.g., D. D. Young and A. Deiters. Org Biomol Chem. 2007:5:999-1005; and Y. Wu. Z. Yang. and Y. Lu. Curr Opin Chem Biol. 2020:57:95-104; the disclosures of which are each incorporated herein by reference). While a few examples are provided, it is understood that any appropriate photo removable group and other nucleobases may be used in accordance with the various embodiments.

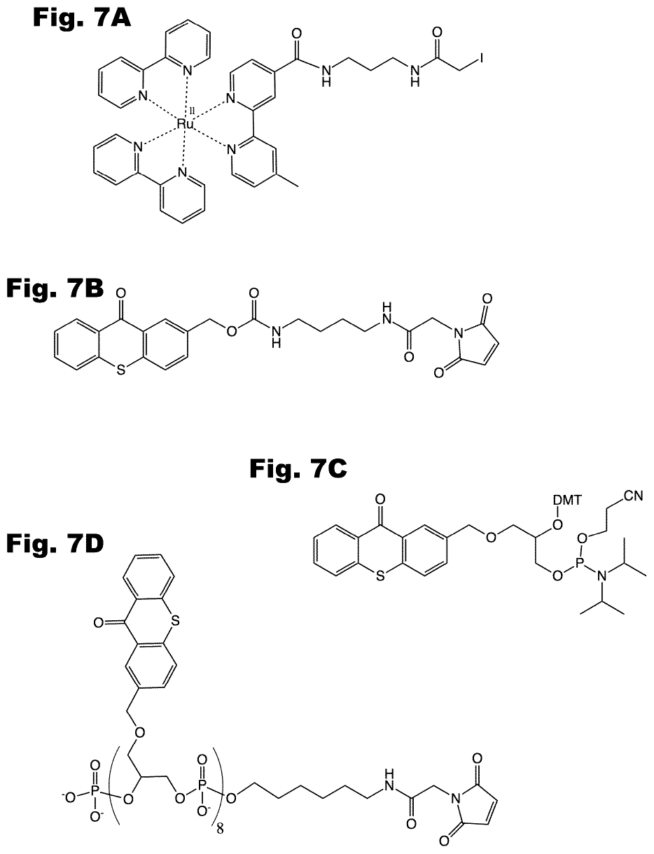

FIG. 6A provides examples of residues having convertible residues. FIG. 6B provides DNA template strands that can be altered by light signals to encode data, in accordance with various embodiments. Note that residues having convertible residues may be spaced apart by non-alterable residues, such as (for example) natural DNA bases. Spacing of convertible residues can help ensure that the enzyme is near only a single convertible residue for pulses of light or redox as the enzyme moves along the template. In many embodiments, the template DNA is prepared with a repeating sequence that regularly spaces the convertible residues such that these groups are independently altered and not altered simultaneously. In several, two convertible residues are positioned very near one another (e.g., within 6 base pairs, such that the groups are utilized in tandem and altered simultaneously by the sensitized enzyme (see FIG. 6B).

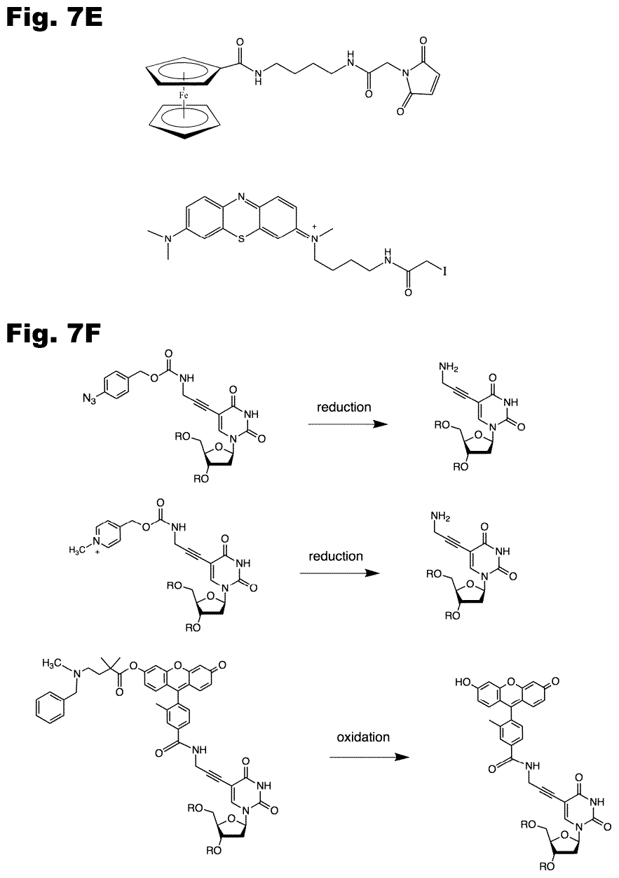

FIGS. 7A to 7F provide examples of sensitizer molecules that can be conjugated to a DNA polymerase enzyme, in accordance with various embodiments. The sensitizer molecules can be used as an antenna to help catalyze chemical alterations. When conjugated to the enzyme, the sensitiver molecule efficiently captures light or redox energy, and then transfers this energy to the convertible residues on the DNA template. The examples provided in FIGS. 7A to 7D are thiol-reactive molecules that can be conjugated to cysteine sidechains, although other types of conjugation methods are known in the art. FIG. 7A provides an example of a Ru-tris (BiPy) sensitizer (S. Pierce, et al., Inorg Chem. 2020:59:14866-14870; and J. B. Edson. L. P. Spencer, and J. M. Boncella. Org Lett. 2011:13:6156-9; the disclosures of which are each incorporated herein by reference); FIG. 7B provides an example of a monomeric thioxanthenone sensitizer (D. Woll, et al., Helv Chim Acta. 2004:87:28-45; and K. A. Ryu, et al., Nat Rev Chem. 2021:5:322-337; the disclosures of which are each incorporated herein by reference); FIG. 7C provides an example of a phosphoramidite monomer that can be used on a DNA synthesizer to assemble an oligomeric sensitizer; FIG. 7D provides an example of an oligomeric antenna that can capture more photons than a single sensitizer can. Although a few examples are provided here any one of multiple types of sensitizers are contemplated. (see e.g., K. A. Ryu, et al., Nat Rev Chem. 2021:5:322-337; and M. Klausen, et al., Chempluschem. 201:84:589-598: the disclosures of which are each incorporated herein by reference)

FIG. 7E provides an example of redox sensitizer structures, which can be conjugated to a polymerase enzyme, either singly or in groups. Application of oxidation or reduction potential via an electrode in solution causes the sensitizer molecule to be oxidized or reduced. This generated redox potential is then transferred to the chemically alterable nearby group in the DNA, resulting in a chemical change. FIG. 7F provides examples of redox-alterable groups that can be incorporated into DNA. These redox-alterable groups can be modified by a nearby redox sensitizer conjugated to a polymerase enzyme as it moves along the DNA template. A number of redox-cleavable linkers can be utilized, many of which are known in the art (see, e.g., R. Camble. R. Garner, and G. T. Young. J Chem Soc C. 1969:1911-1916; and J. B. Edson. L. P. Spencer, and J. M. Boncella. Org Lett. 2011:13:6156-9; the disclosures of which are each incorporated herein by reference)

In many embodiments, to read the data on written nucleic acid polymers, any appropriate sequencer capable of reading unnatural and/or altered nucleobases can be utilized. Examples of commercial nanopore sequencers include Oxford Nanopore Technologies PromethION. MinION, and GridION sequencing platforms (Oxford. UK) and Pacific Bioscience's Single Molecule. Real-Time (SMRT) sequencing platform (Menlo Park. CA). Alternatively, a nanopore device can be fabricated or manufactured for reading the data. The nanopore can be comprised of solid-state materials or can contain one or more proteins.

### Modifiable Fluorophores

Described herein are compositions and systems of data storage utilizing polymers, methods of use and methods of synthesis, in accordance with various embodiments. In several embodiments, a system of data storage comprises writable polymers having one or more convertible residues. In some embodiments, the convertible residues comprise convertible nucleotides. In several embodiments, a system of data storage comprises writable (i.e., data-encodable) polymers having one or more residues that are convertible. Accordingly, a writable nucleic acid polymer is akin to a blank tape that is encodable, wherein the writable nucleic acid polymer is encoded by converting one or more its nucleobases. Conversion of convertible residues can be thought of as a binary code, where each convertible residue is akin to a “bit,” unconverted convertible residues are akin to a “0,” and convertible residues that have been converted are akin to a “1.” It should be understood, however, that a binary code is not the only possibility, and codes can be written in ternary, quaternary, or other numeral system code, which can be done utilizing multiple types of convertible residues or performing multiple writings to further alter the state of a convertible residue. In some embodiments, the conversion of a convertible residue is stable, or permanent, which allows for long-term archiving. In some embodiments, the combination of two convertible residues comprises a “bit”.

In some embodiments, a first fluorescent state comprises a blank state (e.g., unwritten state), a “0” state, or a “1” state. In some embodiments, a second fluorescent state comprises a blank state (e.g., unwritten state), a “0)” state, or a “1” state. In some embodiments, a third fluorescent state comprises a blank state (e.g., unwritten state), a “0)” state, or a “1” state. In some embodiments, a first fluorescent state comprising a blank state may be converted to a second fluorescent state comprising a “1” state. In some embodiments, a first fluorescent state comprising a “1” state may be converted to a second fluorescent state comprising a “0” state.

In some embodiments, the conversion of the convertible residue from a first state to a second state is executed by a writing device. In some embodiments, the writing device comprises a light impinging module (e.g., light source). In some embodiments, the state (e.g., first state, second state, third state, etc.,) is detected by a reading device or unit. In some embodiments, the reading unit comprises the writing device. In some embodiments, the reading unit comprises a (fluorescence) detection device. In some embodiments, the reading unit comprises an analysis module.

In some embodiments, the conversion of the convertible residues comprises any detectable change of fluorescence state, including photoactivation of a fluorophore, inactivation of a fluorophore (e.g., by light), release of a fluorophore, uncaging of a caged fluorophore, quenching of a fluorophore by a quencher (e.g., a quencher release from a convertible residue), and photobleaching of a fluorophore.

In some embodiments, convertible residues may comprise a fluorophore. In some embodiments, the fluorophore may comprise a modifiable fluorophore. In some embodiments, the convertible residue may comprise a leaving group. In some embodiments, the leaving group may be a quencher or a cage (e.g., photo-removeable group or photo-cleavable group). In some embodiments, the fluorophore comprises the leaving group. In some embodiments, the leaving group of the fluorophore may be the cage. In some embodiments, the fluorophore may be a caged fluorophore (e.g., the fluorophore comprising the cage). In some embodiments, the cage may be the leaving group of the modifiable fluorophore. In some embodiments, the convertible residue may be a convertible fluorophore. In some embodiments, the convertible residue comprises the leaving group, wherein the leaving group may be a quencher.

In some embodiments, the convertible residue comprises a modifiable fluorophore that can be activated by light. In some embodiments, the modifiable fluorophore can be activated by light in the presence of an additive (e.g., a phosphine). In some embodiments, the convertible residue comprises a modifiable fluorophore that can be inactivated by light. In some embodiments, the modifiable fluorophore can be inactivated by light in the presence of an additive (e.g., a phosphine).

In some embodiments, the convertible residue comprises a releasable fluorophore that is capable of being released from the polymer by light.

In some embodiments, the convertible residue comprises a photobleachable fluorophore that is capable of being photobleached by light.

Described herein are various compositions, systems, methods of making and methods of use, for a (writable) polymer for encoding data, comprising: a plurality of convertible residues iteratively spaced along and covalently linked to the backbone of the polymer, wherein each convertible residue of the plurality of convertible residues has a first fluorescent state, and is capable of being converted from the first fluorescent state into a second fluorescent state, the first fluorescent state and the second fluorescent state being different: wherein each of all or a subset of the plurality of convertible residues comprises chemically modifiable moiety (e.g., a modifiable fluorophore), and wherein the plurality of convertible residues are covalently linked to the polymer in the first fluorescent state and in the second fluorescent state.

In some embodiments, wherein each of the convertible residues of the plurality of convertible residues are iteratively spaced along the backbone of the polymer, iteratively spaced can be referred to as approximately regularly spaced.

In some embodiments, a convertible residue (e.g., a residue comprising a modifiable fluorophore or a residue comprising a releasable quencher) is referred to as a writable “bit.” and a converted residue (e.g., an altered chemically alterable group or a converted fluorophore with altered emission) is referred to as a written “bit.”

In some embodiment, the terms “writable” and “data-encodable” are used herein interchangeably. In some embodiment, the terms “writing” and “data encoding” are used herein interchangeably.

In some embodiments, the terms “leaving group” and “removable group” are used herein interchangeably. In some embodiments, when referring to convertible residues, the terms “pair” and “duad” are used herein interchangeably. “Duad,” used herein refer to a pair of different convertible residues (e.g., writable bits) that are located close enough relative to one another in the polymers described herein (e.g., nucleic acid polymers) such that both are exposed to a single writing action or event (e.g., the same pulse of light or the same voltage pulse). Thus, the convertible residues that comprise the duad may be closer than the resolution of the writing action or event.

In several embodiments, the one or more convertible residues comprise one or more chemically modifiable moieties (e.g., modifiable fluorophores). Accordingly, a writable polymer is akin to a blank tape that is encodable, wherein the writable polymer is encoded by turning on/off or converting a fluorophore, which can be done by any method in which a fluorophore can be modified. In various embodiments, fluorophores are modified by uncaging, unquenching, and/or photoconverting, depending on the modification mechanism utilized. Fluorophore modification can be thought of as a binary code, where a modifiable fluorophore is akin to a “bit:” one state of a fluorophore is akin to a “0,” and a second state of a fluorophore akin to a “1”. For instance, in one example, a caged fluorophore can be akin to “0” and an uncaged fluorophore can be akin to a “1”. It should be understood, however, that a binary code is not the only possibility, and codes can be written in ternary, quaternary, or other numeral system code, which can be done utilizing multiple types of fluorophores or performing multiple writings/modifications to further alter the state of a fluorophore. The modification of a fluorophore can be stable, or permanent, which allows for long-term archiving, especially if kept in a dark storage location. In some embodiments, the combination of two uniquely identifiable fluorophores comprises a “bit”. For instance, a caged fluorophore and a quenched fluorophore can be utilized to be a single bit, wherein an uncaged fluorophore having a first fluorescent emission intensity or wavelength can be akin to a “0” and an unquenched fluorophore having a second fluorescent emission intensity or wavelength can be akin to a “1”.

### NUMBERED EMBODIMENTS

1. A nucleic acid polymer for encoding data, comprising:
a plurality of residues having a chemically alterable group iteratively spaced along and linked via the nucleic acid polymer backbone, wherein each chemically alterable group of the plurality of convertible nucleobases is provided having a first state and is capable of being altered from the first state into a second state; and
wherein the nucleic acid polymer includes a 3′-tail with a unique sequence for priming a polymerase, wherein the unique sequence of the 3′-tail is only present in the 3′-tail of the nucleic acid polymer.

2. The nucleic acid polymer of embodiment 1 further comprising a plurality of spacer residues linked via the nucleic acid polymer backbone, wherein sets of one or more spacer residues of the plurality of spacer residues are in-between each chemically alterable group of the plurality of residues having the chemically alterable group and provide the iterative spacing among the plurality of the residues having the chemically alterable group.

3. The nucleic acid polymer of embodiment 1 or 2, wherein the iterative spacing among the plurality of the residues having a chemically alterable group conforms to a resolution of a sensitizer for encoding data on the nucleic acid polymer.

4. The nucleic acid polymer of embodiment 1, 2, or 3 further comprising delimiter residues or data tag residues linked to the nucleic acid polymer backbone.

5. The nucleic acid polymer of any one of embodiments 1 to 4, wherein the residues having the chemically alterable group of the plurality of residues having the chemically alterable groups are selected from:

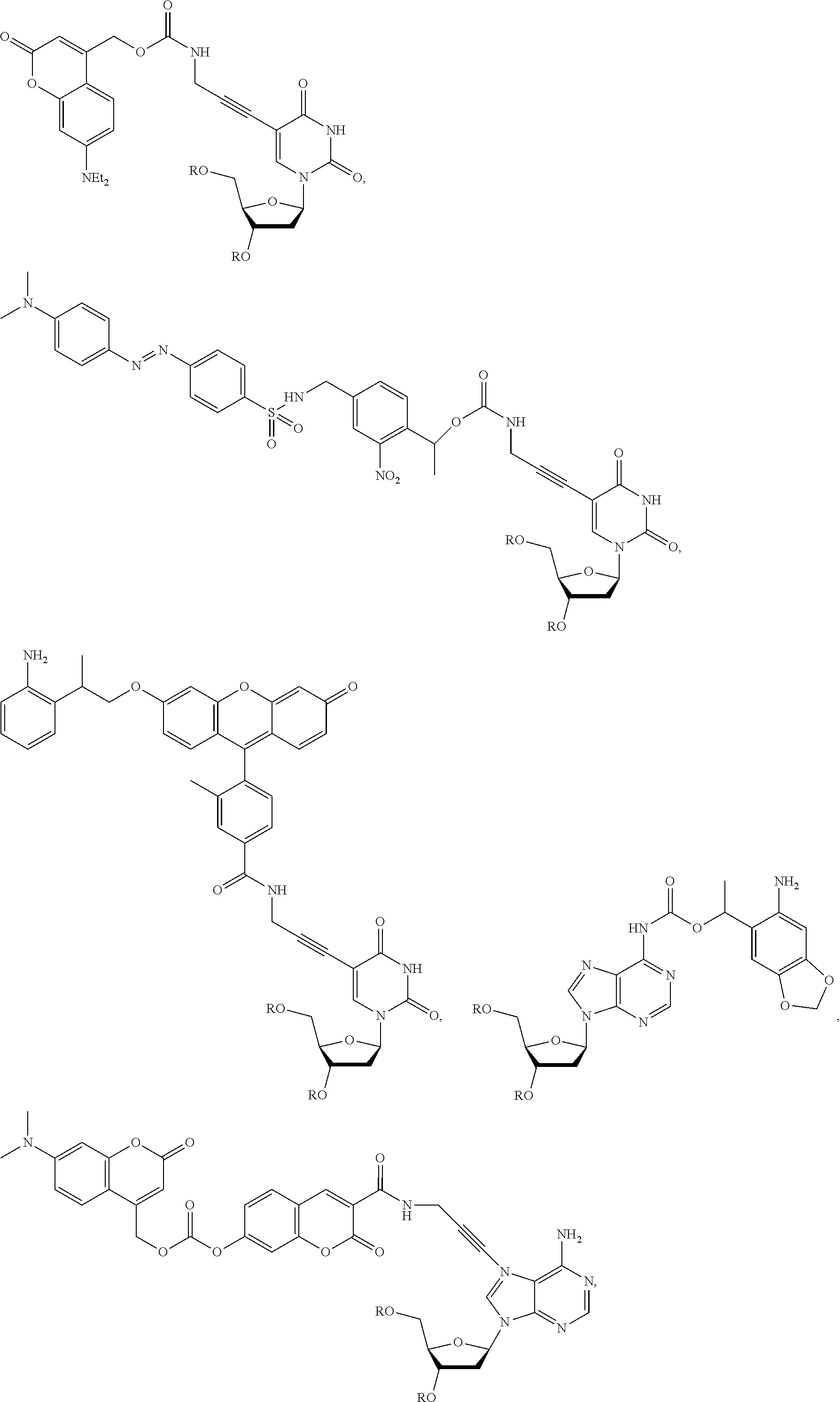

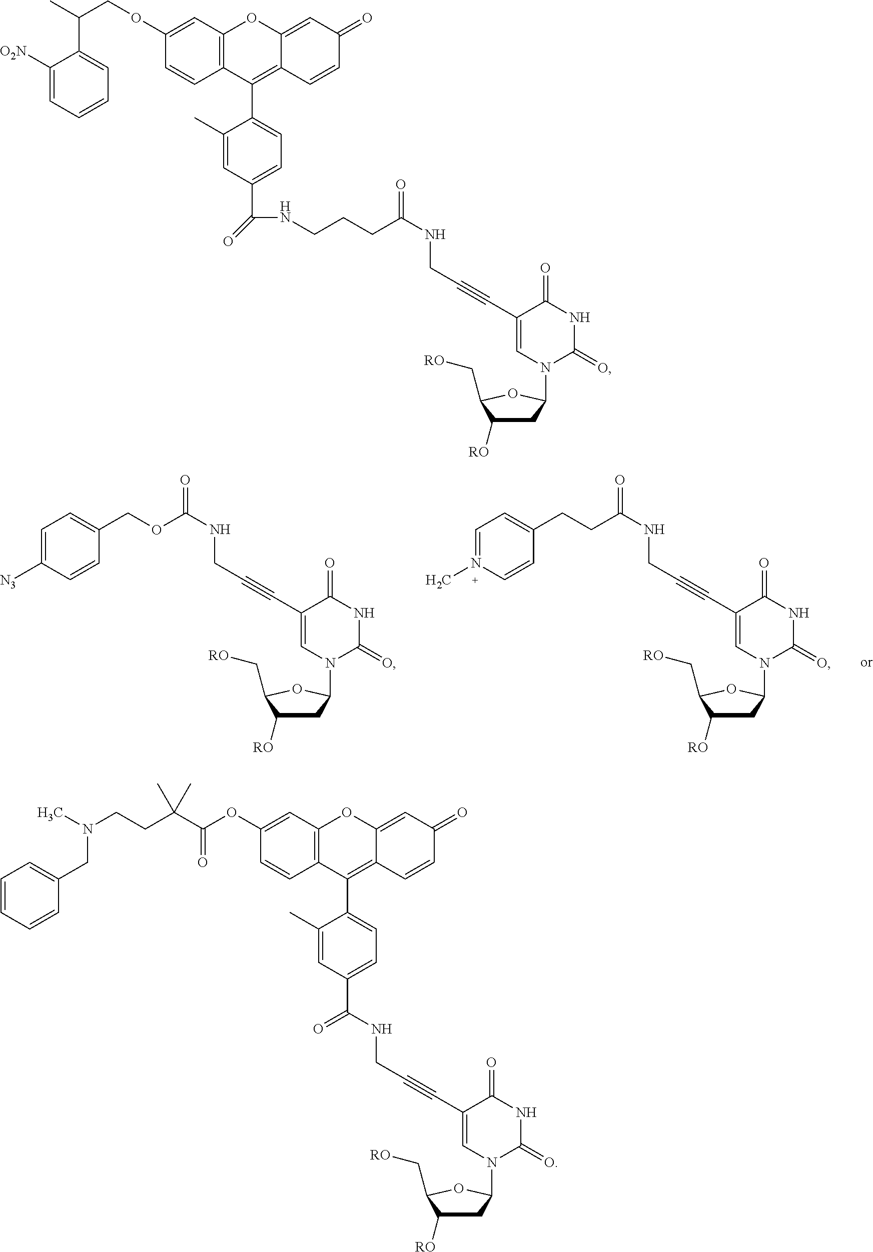

6. The nucleic acid polymer of any one of embodiments 1 to 5, wherein the nucleic acid polymer incorporates residues of DNA, RNA, phosphorothiate DNA, enantio-DNA, glycerol nucleic acids (GNA), threose nucleic acids (TNA), 2′-fluoro-DNA, 2′-O-methyl RNA, or locked nucleic acids (LNA).

7. The nucleic acid polymer of any one of embodiments 1 to 6, wherein the plurality of residues having a chemically alterable group all have the same structure.

8. A polymerase for use in encoding data into a nucleic acid polymer, comprising:
a nucleic acid polymerase conjugated with sensitizer, wherein the sensitizer is molecule that can capture and transmit light or redox energy.

9. The polymer of embodiment 8, wherein the sensitizer is conjugated to the polymerase via cysteine side-chains.

10. The polymerase of embodiment 8 or 9, wherein the sensitizer has a structure selected from:

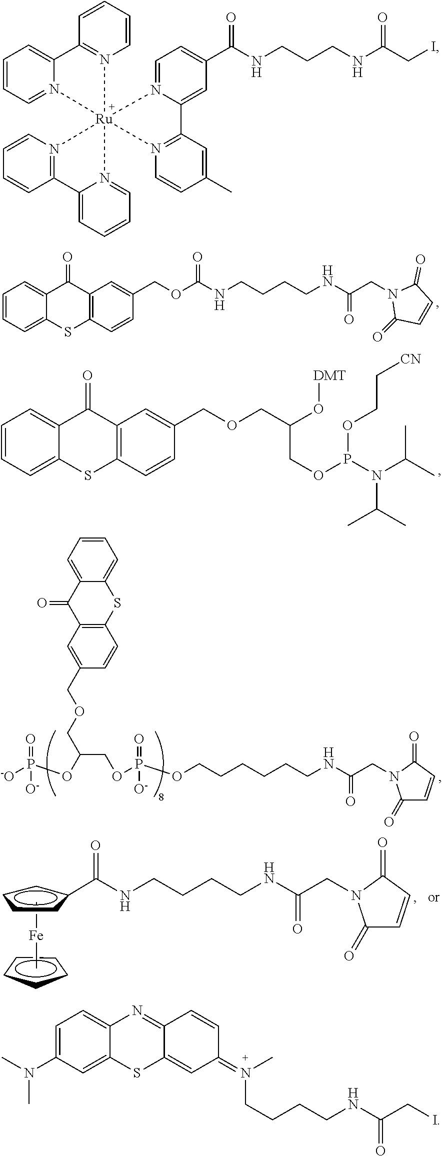

11. A system for data writing into nucleic acids, comprising:
a writable nucleic acid polymer comprising a plurality of residues having a chemically alterable group iteratively spaced along and linked via the nucleic acid polymer backbone, wherein each chemically alterable group of the plurality of convertible nucleobases is provided having a first state and is capable of being altered from the first state into a second state;
an energy source for providing light or redox energy; and
a nucleic acid polymerase conjugated with sensitizer, wherein the sensitizer is molecule that can capture and transmit light or redox energy.

12. The system of embodiment 11, wherein the residues having the chemically alterable group of the plurality of residues having the chemically alterable groups are selected from:

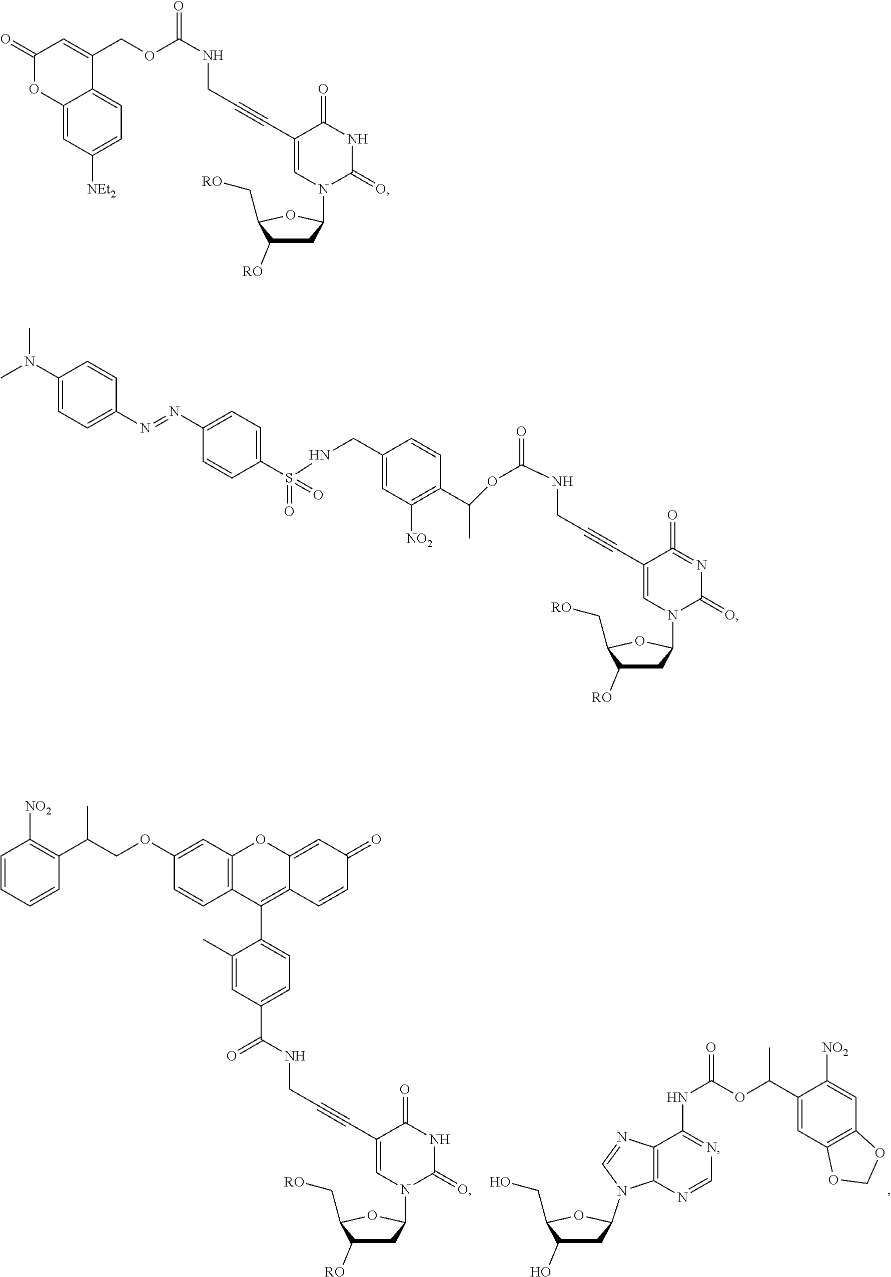

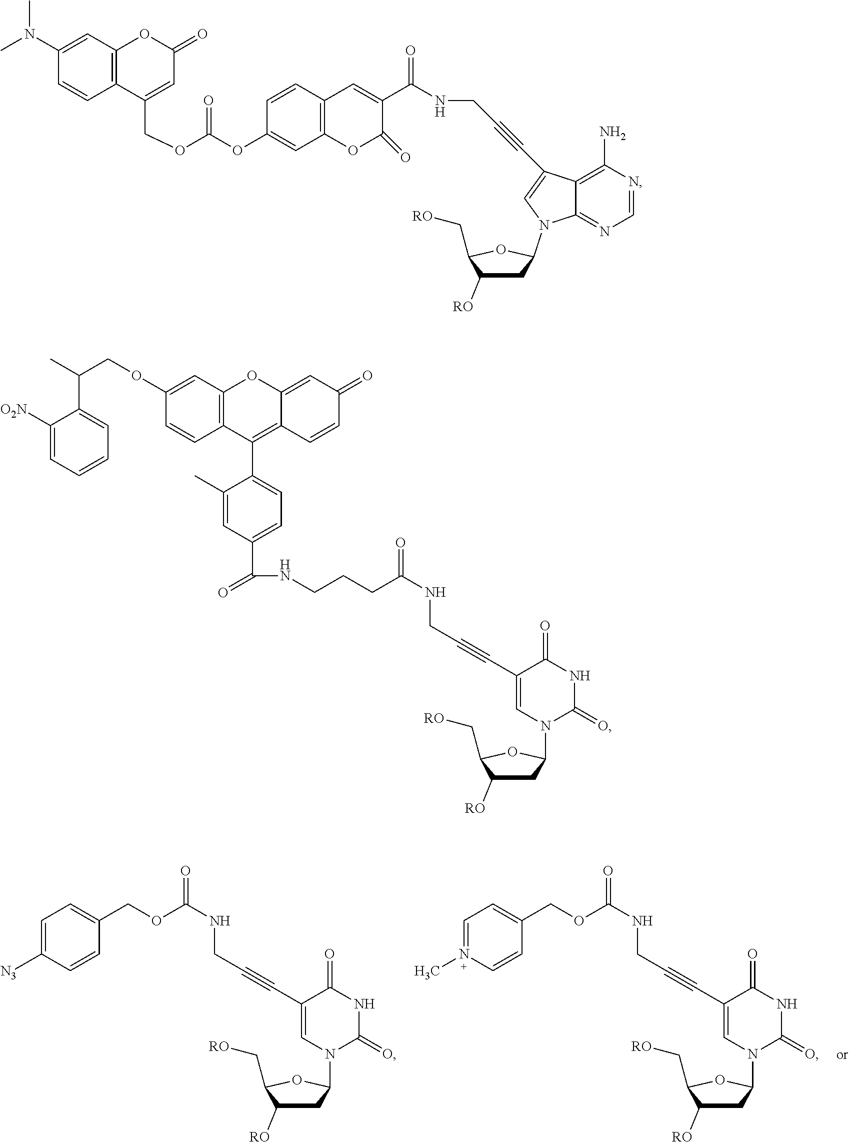

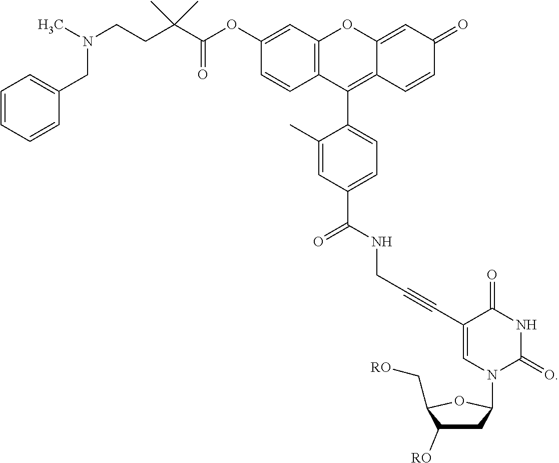

13. The system of embodiment 11 or 12, wherein the nucleic acid polymer further comprises a plurality of spacer residues linked via the nucleic acid polymer backbone, wherein sets of one or more spacer residues of the plurality of spacer residues are in-between each chemically alterable group of the plurality of residues having the chemically alterable group and provide the iterative spacing among the plurality of the residues having the chemically alterable group.

14. The system of embodiment 11, 12 or 13, wherein the iterative spacing among the plurality of the residues having a chemically alterable group conforms to a resolution of a sensitizer for encoding data on the nucleic acid polymer.

15. The system of any one of embodiments 11-14, wherein the plurality of residues having a chemically alterable group all have the same structure.

16. The system of any one of embodiments 11-15, wherein the sensitizer has a structure selected from:

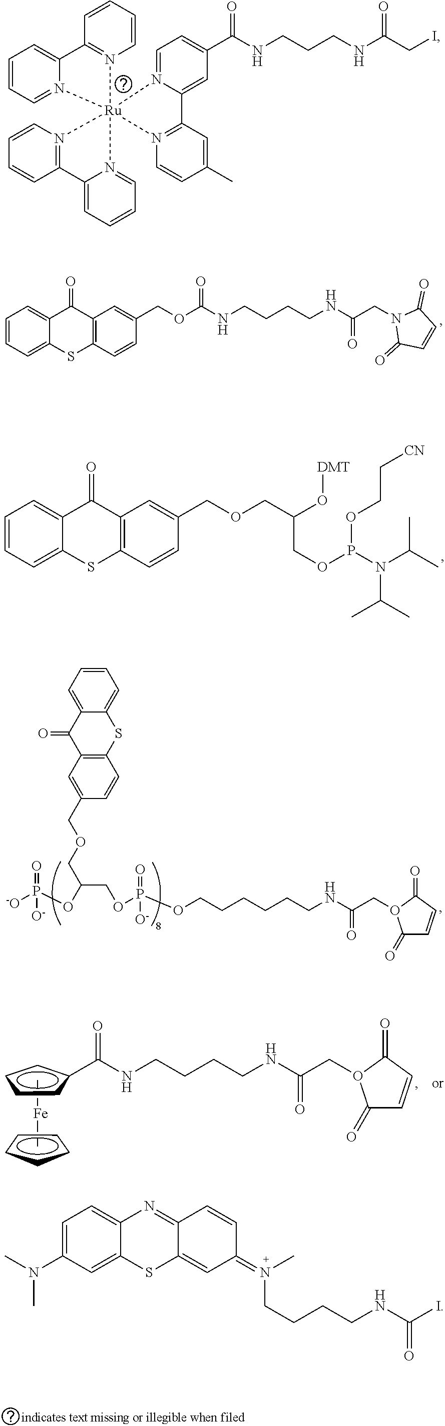

17. The system of any one of embodiments 11-16, wherein the nucleic acid polymer includes a 3′-tail with a unique sequence for priming the nucleic acid polymerase, and wherein the unique sequence of the 3′-tail is only present in the 3′-tail of the nucleic acid polymer.

18. The system of embodiment 17 further comprising a primer oligomer, wherein the primer oligomer has a sequence complementary to the unique sequence of the 3′-tail of the nucleic acid polymer.

19. The system of any one of embodiments 11-18, further comprising triphosphate residues.

20. The system of embodiment 19, wherein the triphosphate residues are dNTPs or NTPs.

21. An encoded nucleic acid polymer, comprising:
a plurality of residues iteratively spaced along and linked via the nucleic acid polymer backbone, wherein each residue of the plurality of residues has an unaltered chemically alterable group or an altered chemically alterable group, and wherein a sequence of unaltered and altered chemically alterable groups represents a code of data.

22. The encoded nucleic acid polymer of embodiment 21, wherein each altered chemically alterable group was altered from an unaltered state into the altered state by a light or redox energy via sensitizer conjugated to a polymerase. 23. The encoded nucleic acid polymer of embodiment 21 or 22, wherein the residues having an unaltered chemically alterable group are selected from:

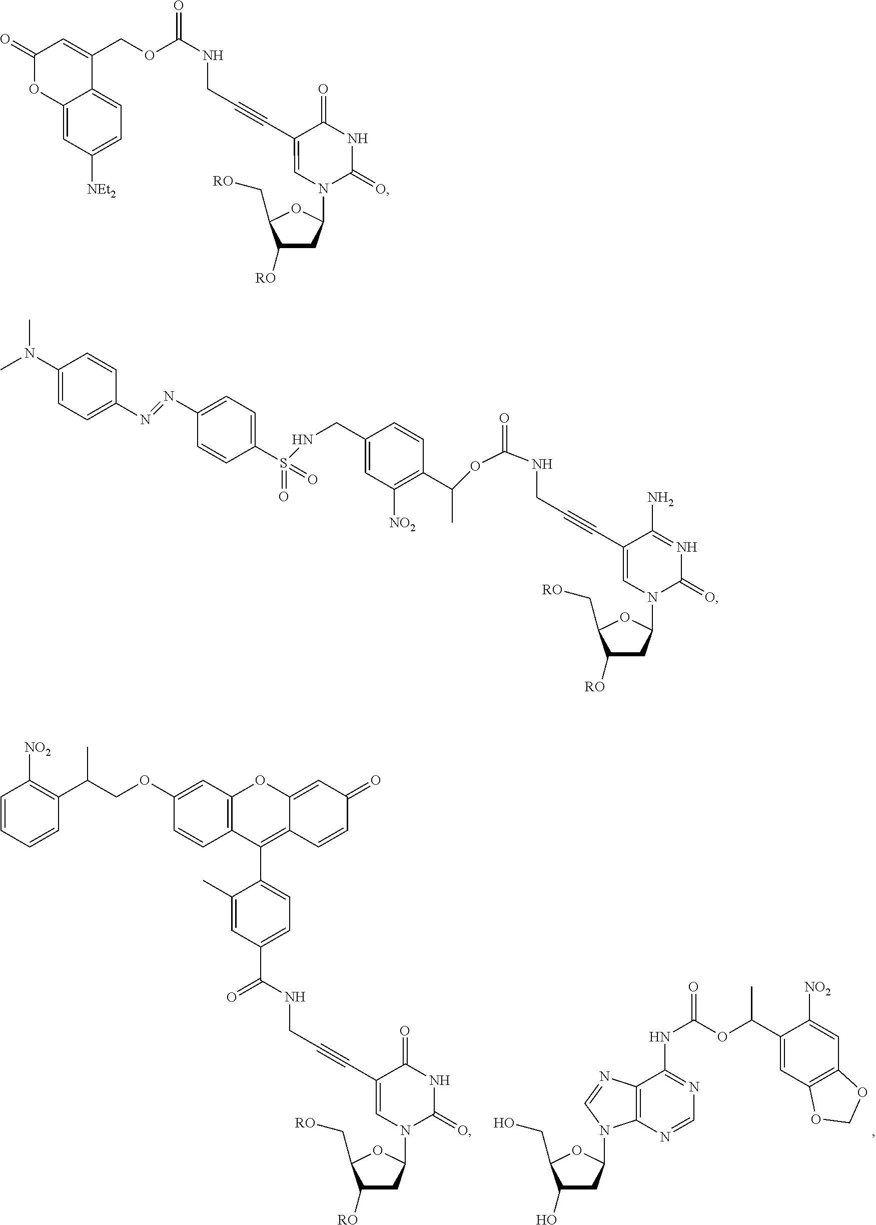

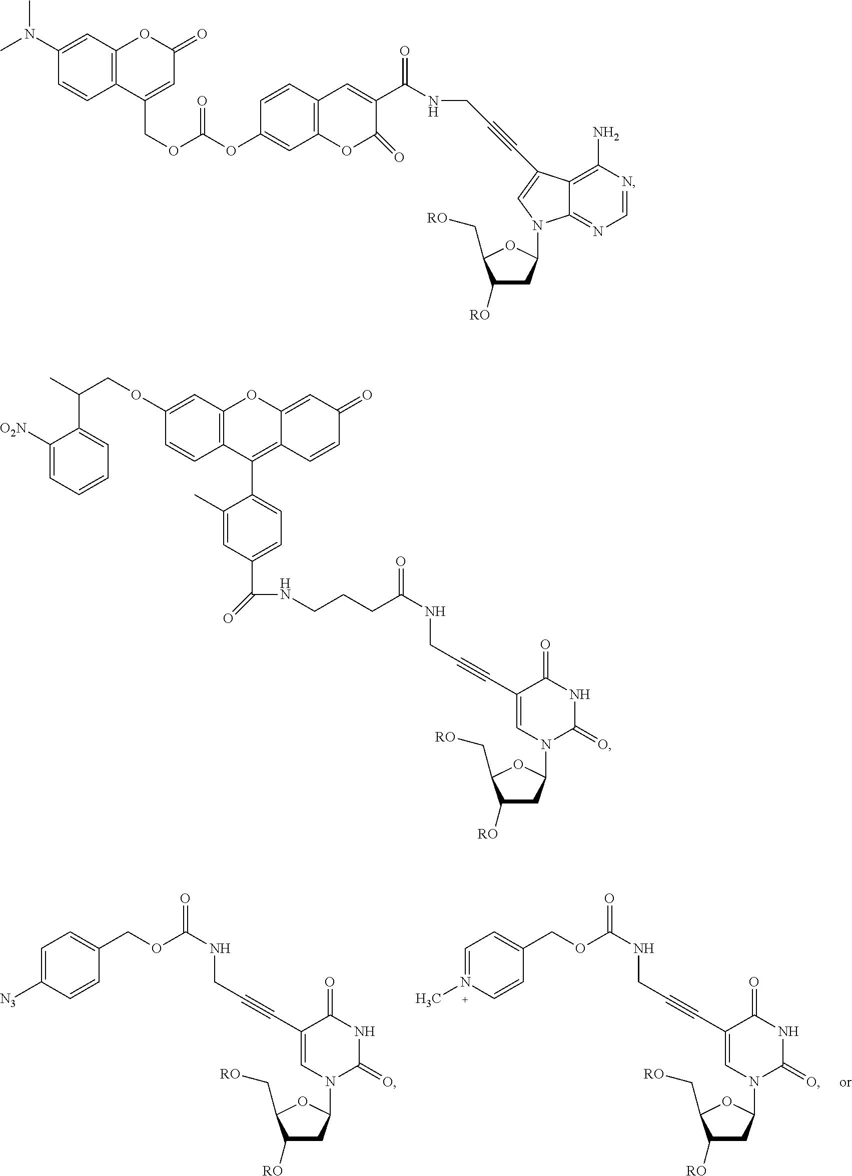

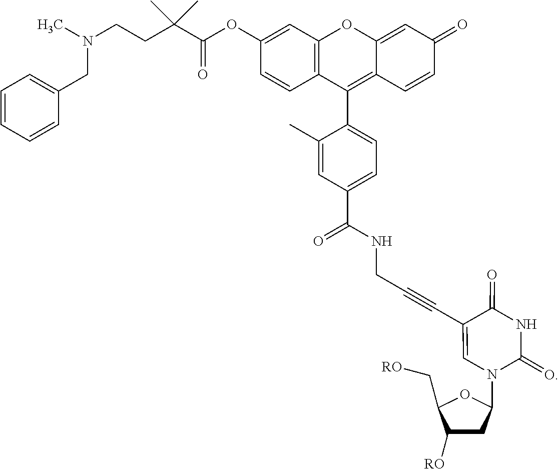

24. The encoded nucleic acid polymer of embodiment 21, 22 or 23, wherein the residues having an unaltered chemically alterable group each have the same structure.

25. The encoded nucleic acid polymer of any one of embodiments 21-24, wherein the residues having an altered chemically alterable group each have the same structure.

26. A method of encoding data onto a writable nucleic acid polymer utilizing light energy, comprising:
providing a solution comprising a writable nucleic acid polymer template that comprises a plurality of residues having a chemically alterable group iteratively spaced along and linked via the nucleic acid polymer backbone, wherein each chemically alterable group of the plurality of convertible nucleobases is provided having a first state and is capable of being altered from the first state into a second state;
adding to the solution a nucleic acid polymerase conjugated with sensitizer molecule; and
selectively pulsing light energy, wherein the pulsed light energy is captured by the sensitizer molecule and transmitted from the sensitizer molecule to residues having the chemically alterable group nearby the sensitizer molecule as the nucleic acid polymerase travels along the writable nucleic acid polymer template, resulting in altering a subset of the plurality of chemically alterable groups into the second state such that a data encoded nucleic acid polymer is generated.

27. The method of embodiment 26, wherein the residues having the chemically alterable group of the plurality of residues having the chemically alterable groups are selected from:

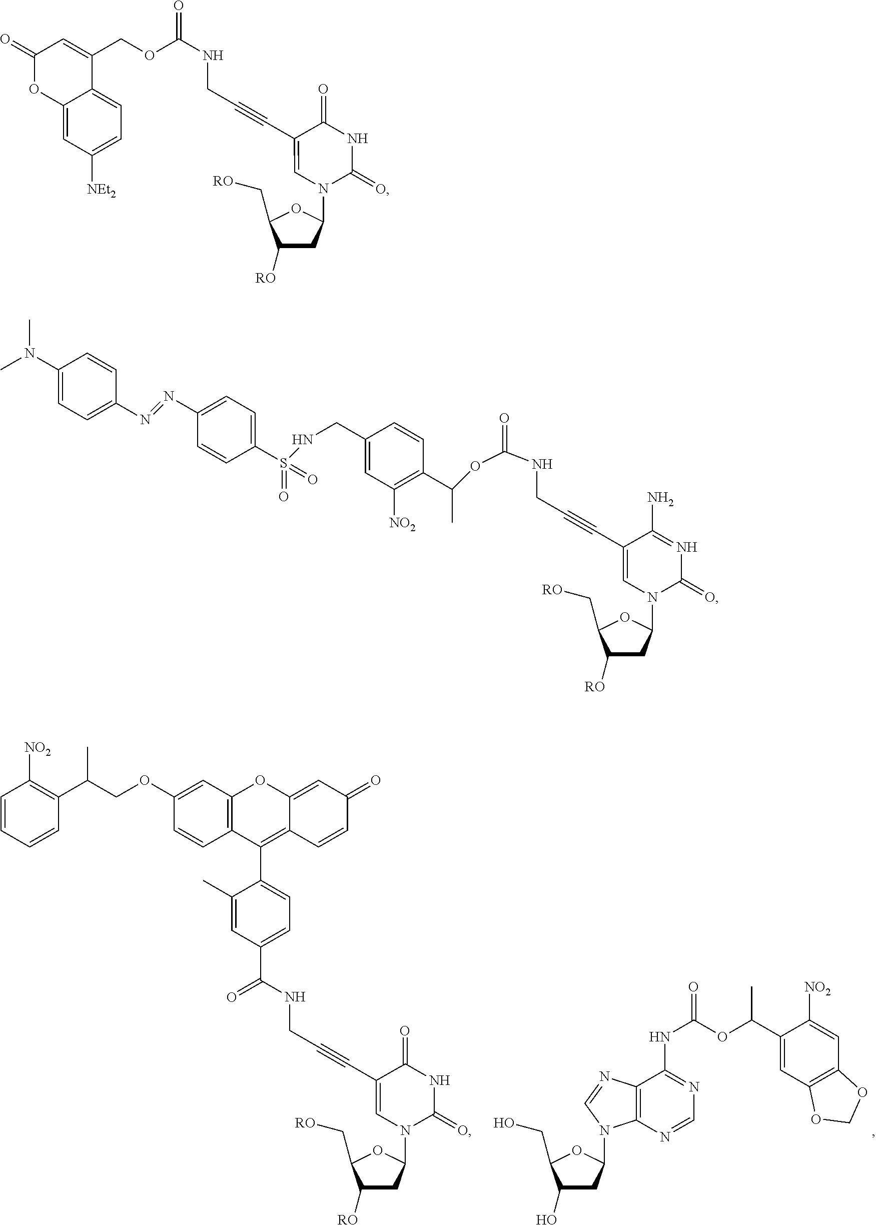

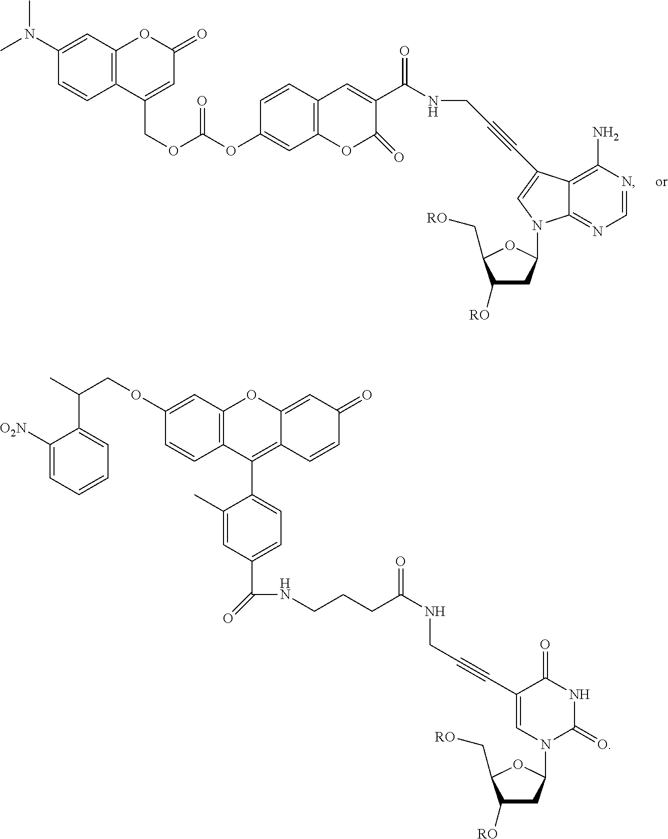

28. The method of embodiment 26 or 27, wherein writable nucleic acid polymer template further comprises a plurality of spacer residues linked via the nucleic acid polymer backbone, wherein sets of one or more spacer residues of the plurality of spacer residues are in-between each chemically alterable group of the plurality of residues having the chemically alterable group and provide the iterative spacing among the plurality of the residues having the chemically alterable group.

29. The method of embodiment 26, 27, or 28, wherein the sensitizer molecule has a structure selected from:

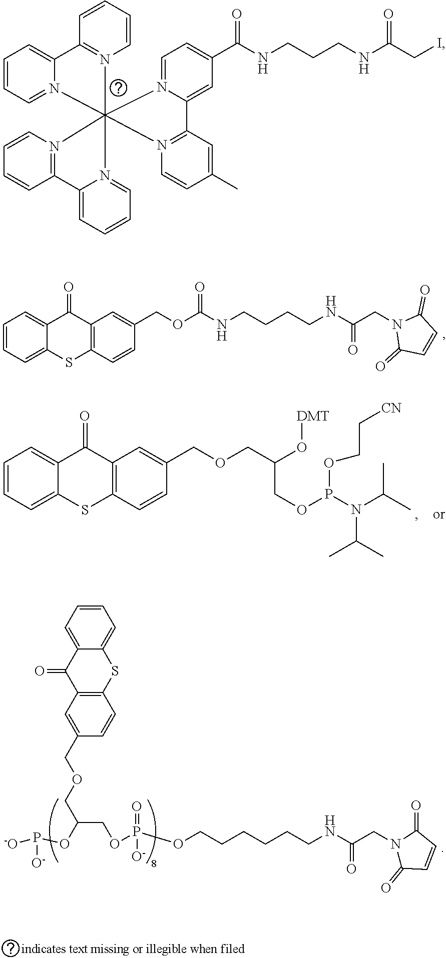

30. The method of any one of embodiments 26-29, wherein writable nucleic acid polymer template includes a 3′-tail with a unique sequence for priming the nucleic acid polymerase, and wherein the unique sequence of the 3′-tail is only present in the 3′-tail of the nucleic acid polymer.

31. The method of embodiment 30 further comprising: adding to the solution a primer oligo, wherein the primer oligomer has a sequence complementary to the unique sequence of the 3′-tail of the nucleic acid polymer.

32. The method of any one of embodiments 26-31 further comprising:
adding to the solution a set of triphosphate residues, wherein the set of triphosphate residues comprises triphosphate residues having a first structure and triphosphate residues having a second structure.

33. The method of embodiment 32, wherein the set of triphosphate residues are added such that a final concentration of triphosphate residues having the first structure is lower than a final concentration of triphosphate residues having the second structure.

34. The method of embodiment 33, wherein the ratio of the triphosphate residues having the first structure to the triphosphate residues having the second structure results in the polymerase pausing as it travels along writable nucleic acid polymer template when it reaches residues of the template complementary to the triphosphate residues having the first structure.

35. A method of encoding data onto a writable nucleic acid polymer utilizing redox potential, comprising:
providing a solution comprising a writable nucleic acid polymer template that comprises a plurality of residues having a chemically alterable group iteratively spaced along and linked via the nucleic acid polymer backbone, wherein each chemically alterable group of the plurality of convertible nucleobases is provided having a first state and is capable of being altered from the first state into a second state;
adding to the solution a nucleic acid polymerase conjugated with sensitizer molecule; and selectively providing redox potential with an electrode in contact with the solution, wherein the redox potential is captured by the sensitizer molecule and transmitted from the sensitizer molecule to residues having the chemically alterable group nearby the sensitizer molecule as the nucleic acid polymerase travels along the writable nucleic acid polymer template, resulting in altering a subset of the plurality of chemically alterable groups into the second state such that a data encoded nucleic acid polymer is generated.

36. The method of embodiment 35, wherein the residues having the chemically alterable group of the plurality of residues having the chemically alterable groups are selected from:

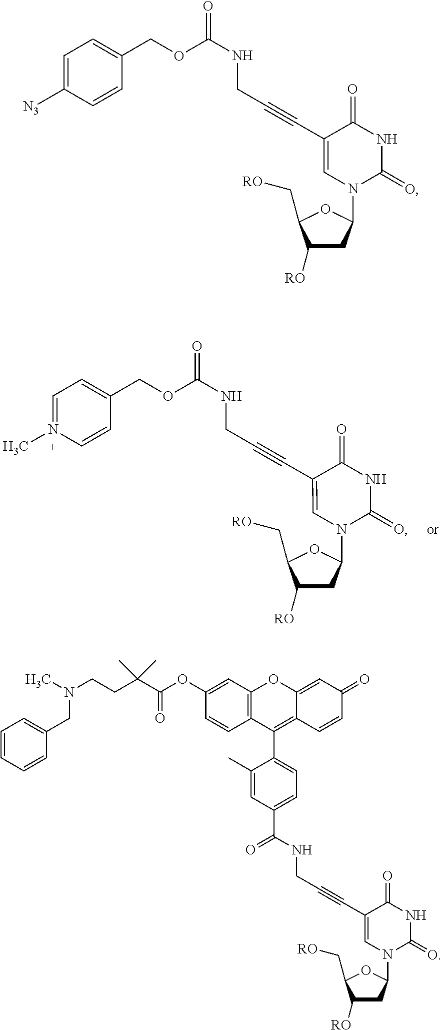

37. The method of embodiment 35 or 36, wherein writable nucleic acid polymer template further comprises a plurality of spacer residues linked via the nucleic acid polymer backbone, wherein sets of one or more spacer residues of the plurality of spacer residues are in-between each chemically alterable group of the plurality of residues having the chemically alterable group and provide the iterative spacing among the plurality of the residues having the chemically alterable group.

38. The method of embodiment 35, 36, or 37, wherein the sensitizer molecule has a structure selected from:

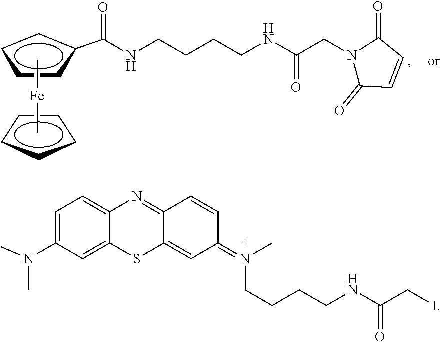

39. The method of any one of embodiments 35-38, wherein writable nucleic acid polymer template includes a 3′-tail with a unique sequence for priming the nucleic acid polymerase, and wherein the unique sequence of the 3′-tail is only present in the 3′-tail of the nucleic acid polymer.

40. The method of embodiment 39 further comprising:
adding to the solution a primer oligo, wherein the primer oligomer has a sequence complementary to the unique sequence of the 3′-tail of the nucleic acid polymer.

41. The method of any one of embodiments 35-40 further comprising:
adding to the solution a set of triphosphate residues, wherein the set of triphosphate residues comprises triphosphate residues having a first structure and triphosphate residues having a second structure.

42. The method of embodiment 41, wherein the set of triphosphate residues are added such that a final concentration of triphosphate residues having the first structure is lower than a final concentration of triphosphate residues having the second structure.

43. The method of embodiment 42, wherein the ratio of the triphosphate residues having the first structure to the triphosphate residues having the second structure results in the polymerase pausing as it travels along writable nucleic acid polymer template when it reaches residues of the template complementary to the triphosphate residues having the first structure.

### EXAMPLES

Described herein are various examples of compositions, systems, and methods for data storage utilizing polymers. Examples of writable nucleic acid polymers, methods to produce such polymers, methods to writing data, and methods for reading data are provided.

### Example 1: Preparation of a DNA Polymerase Modified with One or More Photosensitizer Groups

In order to selectively focus excitation energy on a convertible residue on a DNA polymer, a polymerase is conjugated with a photosensitizer group. The DNA polymerase BST is prepared as a cysteine mutant at position 845, in a structural domain near the DNA when it is bound. It is reacted with a maleimide conjugate of thioxanthenone as the photosensitizer group (see FIG. 7B), and the conjugated enzyme is purified away from the excess sensitizer by size exclusion spin column. In a separate experiment, the polymerase Cys mutant is reacted with the octamer sensitizer reagent of FIG. 7D. Mass spectrometry of the protein reveals the added mass of the conjugated groups for these two preparations, and absorption spectroscopy confirms the addition of the absorbance of the sensitizer group or groups in both cases. Tests with copying a DNA template in the presence of a primer. Mg²⁺, and dNTPs confirm that the modified enzymes remain active in synthesis of DNA.

### Example 2: Use of Light Pulses and a Sensitized Polymerase to Make Structural Changes in DNA During Template Copying

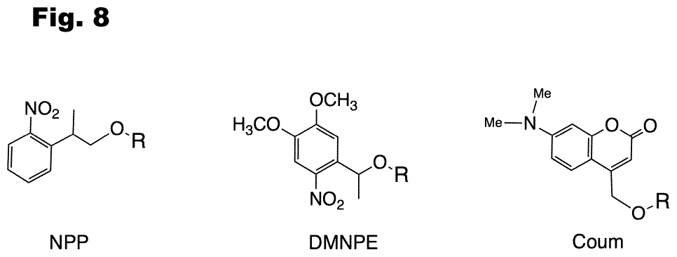

A DNA template containing multiple photocaging groups in a repeating sequence is prepared prior to the experiment. The photocaging group. NPP (FIG. 8), is linked on to aminopropynyl sidechains of deoxyuridine in the sequence. At the 3′-end of the DNA template is a poly(dT) tail, providing a unique site for a primer to bind. In the solution (a pH 7.5 buffer containing 5 mM MgCl2), a poly(dA) primer, and the sensitizer-modified DNA polymerase are added to the template. The template-copying reaction is initiated by the addition of template-complementary dNTPs (200 uM each). The solution is then exposed to five 100 millisecond pulses of light at 380-420 nM, with spacings of 10 seconds between illumination. Control experiments contain polymerase lacking the sensitizer group. The experiment is repeated at different intensities of light, with the goal achieving little or no uncaging of the template DNA when the polymerase has no sensitizer but uncaging at sites along the DNA when it does. Experiments with optimal light intensity results in loss of five caging groups from the DNA template when it is copied with the sensitized polymerase. Further experiments using four or three pulses of light reveal loss of four and three groups from the DNA, respectively. Further experiments with altered timing between pulses reveal ideal timing that is not too short, thus missing an effective bit writing step, or too long, skipping many unwritten bits.

### Example 3: Use of Light Pulses and a Sensitized Polymerase to Make Optical Changes in DNA During Template Copying

A DNA template containing multiple photocaged fluorophores attached at thymidines in the polymer is prepared prior to the experiment. The photocaging group. DMNPE (FIG. 8), is present on the fluorophores, rendering them dark. At the 3′-end of the DNA template is a poly(dA) tail, providing a unique site for a primer to bind. In the solution (a pH 7.5 buffer containing 5 mM MgCl₂), a poly(dT) primer, and the sensitizer-modified DNA polymerase are added to the template. The template-copying reaction is initiated by the addition of template-complementary dNTPs (500 uM each). The solution is then exposed to five 100 millisecond pulses of light at approximately 380-420 nM, with spacings of 10 seconds between illumination. Control experiments contain polymerase lacking the sensitizer group. The experiment is repeated at different intensities of light, with the goal achieving little or no uncaging of the template DNA fluorophores when the polymerase has no sensitizer but uncaging at sites along the DNA when it does. Experiments with optimal light intensity results in release of five caging groups from the DNA template when it is copied with the sensitized polymerase, which can be observed by fluorescence and by mass spectrometry. Further experiments using four or three pulses of light reveal loss of four and three groups from the DNA, respectively, uncaging four and three fluorophore groups.

### Example 4: Use of Dual Wavelength Light Pulses and a Sensitized Polymerase to Make Dual Optical Changes in DNA During Template Copying

A DNA single-stranded template containing multiple dual photocaged coumarin and Tokyo green fluorophores at nearby nucleotides in the sequence is prepared prior to the experiment. The photocaging groups, NPP and Coum (FIG. 8), are linked to the fluorophores, rendering them dark. At the 3′-end of the DNA template is a poly(dT) tail, providing a unique site for a primer to bind. In the solution (a pH 7.5 buffer containing 5 mM MgCl₂), a poly(dA) primer, and a sensitizer-modified DNA polymerase are added to the template. In this case, the polymerase is modified with two sensitizers. Ru (tris) BiPy and thioxanthenone. The template-copying reaction is initiated by the addition of template-complementary dNTPs (500 μM each). The solution is then exposed to 100 millisecond pulses of LED light, with spacings of 10 seconds between illumination. Control experiments contain polymerase lacking the sensitizer group. The experiment is repeated at different intensities of light, with the goal achieving little or no uncaging of the template DNA fluorophores when the polymerase has no sensitizer but uncaging at sites along the DNA when it does. Experiments with optimal light intensity results in loss of caging groups from the DNA template when it is copied with the sensitized polymerase, which can be observed by fluorescence and by mass spectrometry. Further experiments using only 500 nm light pulses results in loss of Coum caging groups, resulting in blue fluorescence at the polymerase-sensitized sites. Conversely, use of 380 nm light results in uncaging of both the Coum and NPP groups, resulting in fluorescence in both fluorophores. Because they are nearby, some of the coumarin fluorescence energy is donated to the nearby Tokyo green dye, enhancing its fluorescence. In a third experiment, the pulses are performing in alternating fashion with 500 nm and 365 nm light. This results in alternating outcomes of uncaging, and this results in blue fluorescence and green fluorescence emission alternating along the written DNA strand.

### Example 5: Use of an Electrode and a Redox-Sensitized Polymerase to Make Structural Changes in DNA

A DNA template containing multiple redox-sensitive caging groups having a repeating sequence is prepared prior to the experiment. The redox-sensitive group, picolylmethyl, (FIG. 5A), is linked to an aminopropynyl sidechain of deoxyuridine in the sequence. At the 3′-end of the DNA template is a poly(dT) tail, providing a unique site for a primer to bind. In the solution (a pH 7.5 buffer containing 5 mM MgCl₂), a poly(dA) primer, and a redox sensitizer-modified DNA polymerase are added to the template. The template-copying reaction is initiated by the addition of template-complementary dNTPs (200 μM each). The solution is then exposed to five 100 millisecond pulses of voltage potential at a potential sufficient to reduce methylene blue, with spacings of 10 seconds between the pulses (J. D. Mahlum, M. A. Pellitero, and N. Arroyo-Curras, J Phys Chem C. 2021; 125; 9038-9049; and D. Kang, et al., Anal Chem. 2009; 81; 9109-13; the disclosures of which are each incorporated herein by reference). Control experiments contain polymerase lacking the sensitizer group. The experiment results in loss of five caging groups from the DNA template when it is copied with the sensitized polymerase. Further experiments using four or three voltage pulses result in loss of four and three groups from the DNA, respectively.

### Example 6: Use of a DNA Synthesizer to Make an Oligomeric Sensitizer

A monomeric photosensitizer based on thioxanthenone is prepared with a dimethoxytrityl group on the primary hydroxyl group and a phosphoramidite group on the secondary hydroxyl (FIG. 4C). This monomer is dissolved in dry acetonitrile at 0.1M and placed in a monomer reagent port on a DNA synthesizer. The synthesizer is further supplied with 3′-phosphate-ON solid support. The reagent is then used to synthesize an octamer oligomer using the standard DNA synthesis cycle. If coupling yields are low, an extended coupling time is used. At the last step of synthesis, a 5′-amine phosphoramidite reagent (with TFA protecting group) is coupled. The final oligomer is deprotected with ammonia following standard DNA deprotection protocols and is purified by HPLC and characterized by mass spectrometry. This product is reacted with maleimide NHS ester to provide a thiol-reactive compound ready for derivatization of a cysteine group on a DNA polymerase mutant (see structure in FIG. 4D).

### Example 7: Preparing Modifiable DNA for Polymerase Copying and Pulsed Light Modification

A 50nt circular DNA oligonucleotide is prepared with the repeating sequence (GTTATTTTTT) 5. It encodes a repeating DNA polymer of sequence (AAAAAATAAC) n. Two nucleotides comprising caged fluorophores are further prepared for the experiment: a deoxyuridine substituted with Coum-caged coumarin, and a deoxcytidine substituted with DNPE-caged Tokyo green (FIG. 8). These are prepared in nucleoside triphosphate form to provide the two caged nucleotides. A complementary primer, unmodified BST3.0 DNA polymerase, dATP and the two caged nucleoside triphosphates are incubated in a polymerase-supportive buffer. This results in rolling-circle synthesis of a long repeating DNA single strand containing the two modified nucleotides in each repeat unit. PAGE gel analysis confirms lengths of 1000 nucleotides or more in the product modified strands

1.-103. (canceled)

104. A method comprising:
(a) providing a polymer, wherein said polymer comprises a plurality of convertible residues, wherein each of said convertible residues is in a first state, and is capable of being converted into a second state;
(b) contacting said polymer with an enzyme under conditions to promote binding of said enzyme to said polymer, wherein said enzyme comprises a sensitizer; and
(c) enzymatically encoding data into said polymer with aid of said enzyme by converting at least a subset of convertible residues of the plurality of convertible residues from said first state into the second state, wherein said converting comprises applying a stimulus to said sensitizer to effect conversion of said at least said subset of convertible residues of said plurality of convertible residues from said first state into said second state.

105. The method of claim 104, wherein said polymer comprises a nucleic acid.

106. The method of claim 104, wherein said enzyme comprises a polymerase.

107. The method of claim 106, wherein said polymerase is a deoxyribonucleic acid polymerase.

108. The method of claim 104, wherein said enzyme comprises a helicase.

109. The method of claim 104, wherein said enzyme comprises a nickase.

110. The method of claim 104, wherein said stimulus comprises light pulses.

111. The method of claim 104, wherein said stimulus comprises redox energy.

112. The method of claim 104, wherein said sensitizer is conjugated to said enzyme.

113. The method of claim 112, where said sensitizer is conjugated to said enzyme via a cysteine sidechain.

114. The method of claim 104, wherein a convertible residue of said plurality of convertible residues comprises one of the following:

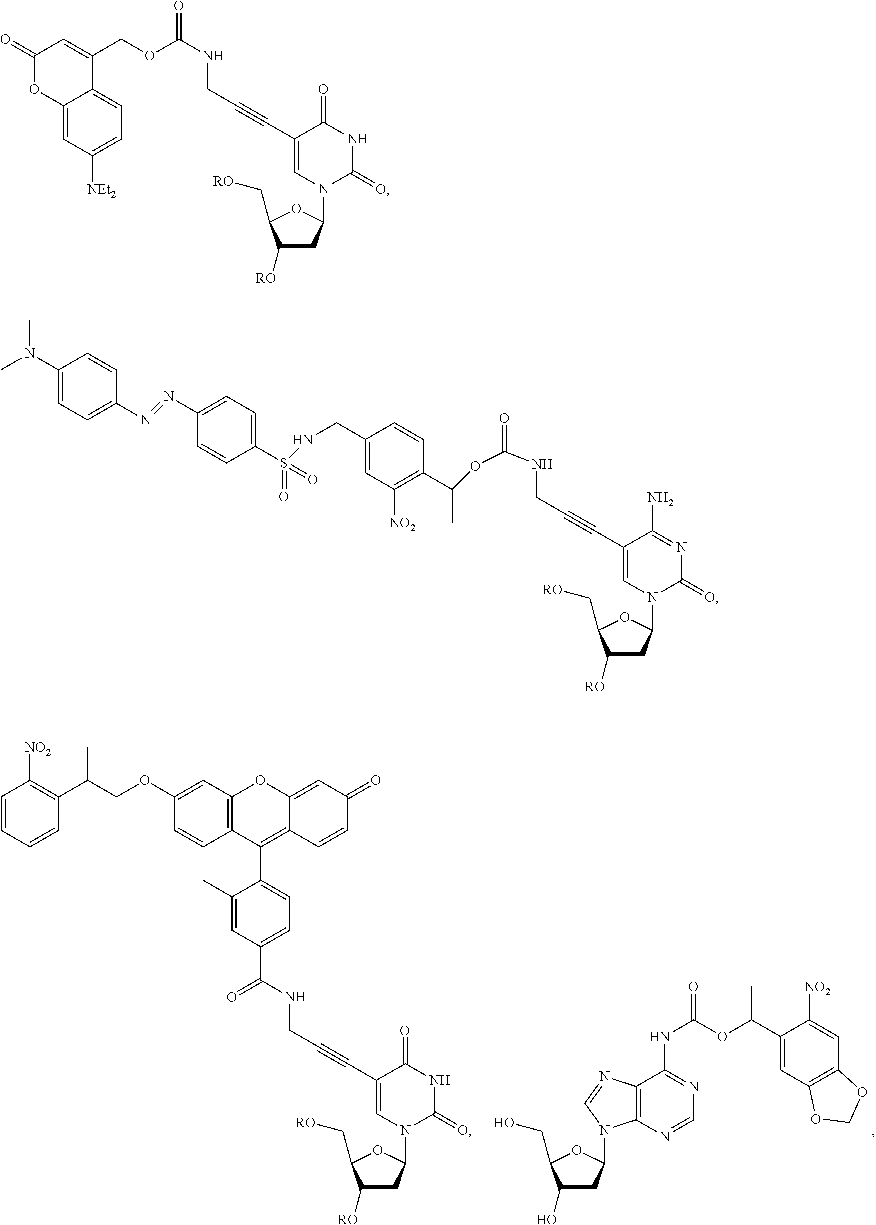

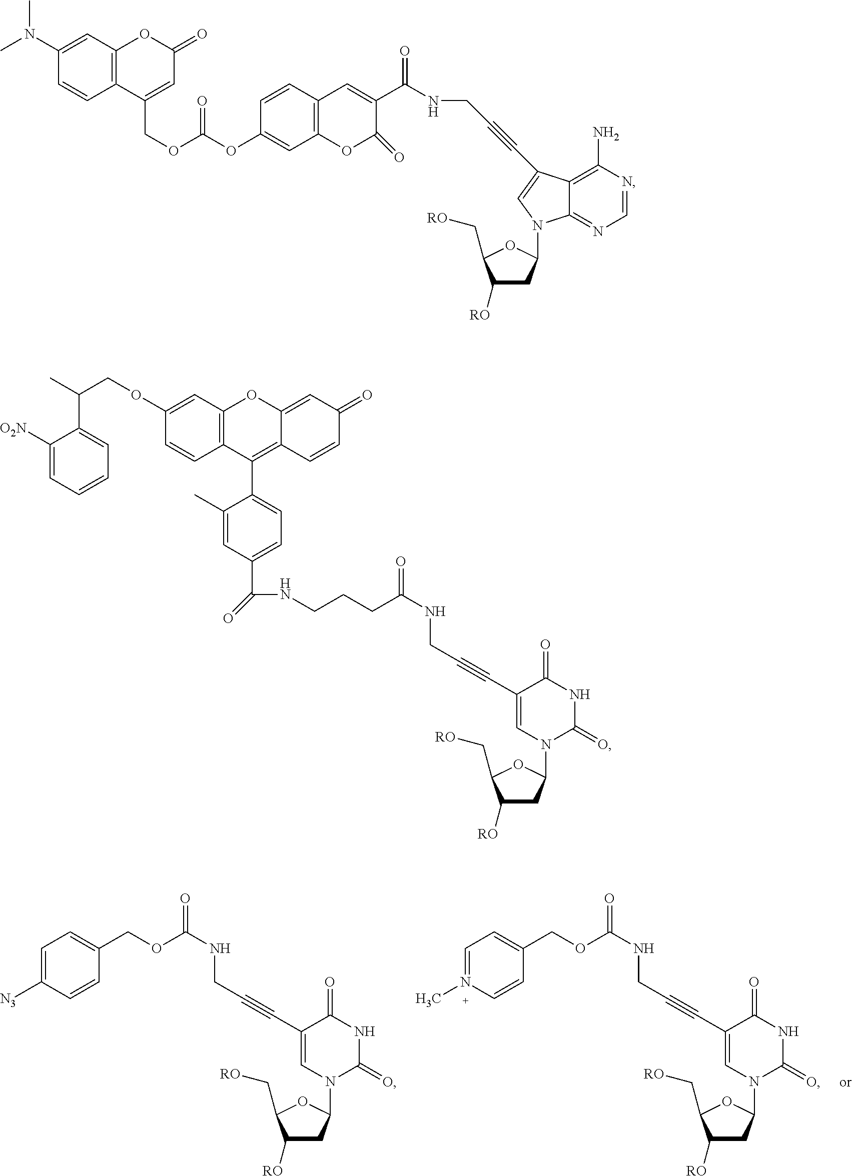

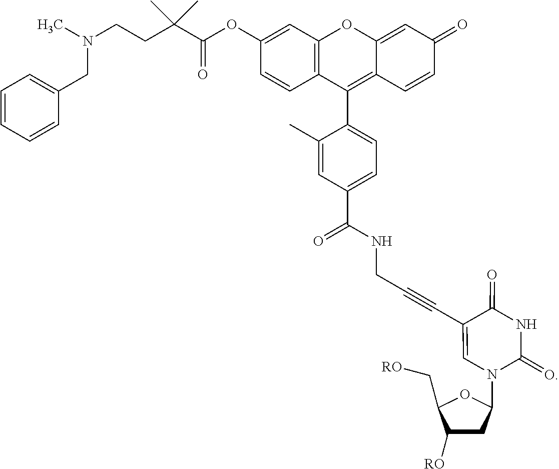

115. The method of claim 104, wherein said sensitizer has a structure of one of the following:

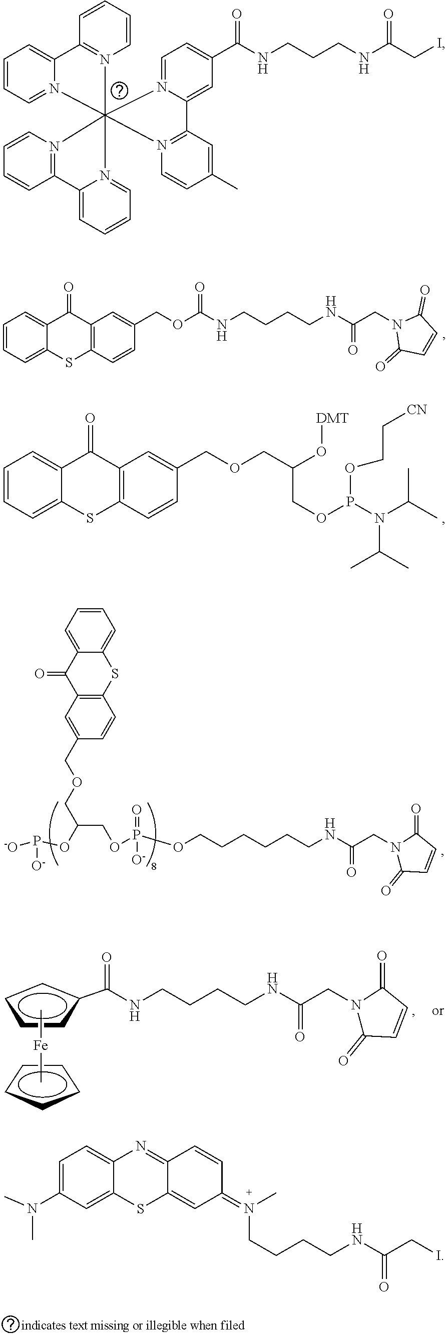

116. The method of claim 104, wherein said polymer further comprises a plurality of spacer residues, wherein each of said spacer residues of said plurality of spacer residues is not capable of being converted into the second state by said stimulus.

117. The method of claim 116, wherein each of said convertible residues of said plurality of convertible residues is separated by 2-50 spacer residues of said plurality of spacer residues.

118. The method of claim 104, wherein said plurality of convertible residues comprises at least 100 convertible residues.

119. The method of claim 104, wherein said plurality of convertible residues comprises at least 1,000 convertible residues.

120. The method of claim 104, wherein (c) comprises using a nanopore to provide said stimulus.

121. The method of claim 120, wherein said nanopore is a plasmonic nanopore.
